(19) United States
(12) Patent Application Publication
(10) Pub. No.: US 2022/0275455 A1
(43) Pub. Date: Sep. 1, 2022

(54) DATA PROCESSING AND CLASSIFICATION FOR DETERMINING A LIKELIHOOD SCORE FOR BREAST DISEASE

(71) Applicant: Preferred Networks, Inc., Chiyoda-ku, Tokyo (JP)

(72) Inventors: Nobuyuki OTA, Burlingame, CA (US); Sandeep AYYAR, Burlingame, CA (US); Timothy J. NOLAN, Burlingame, CA (US)

(73) Assignee: Preferred Networks, Inc., Chiyoda-ku, Tokyo (JP)

(21) Appl. No.: 17/624,950

(22) PCT Filed: Jul. 7, 2020
(86) PCT No.: PCT/JP2020/026597

§ 371 Date: Jan. 5, 2022

Related U.S. Application Data
(60) Provisional application No. 62/871,604, filed on Jul. 8, 2019.

(51) Int. Cl. C12Q 1/6886; G16B 20/00; G16B 40/20; C12Q 1/6806; C12Q 1/6862

(52) U.S. Cl. CPC C12Q 1/6886; G16B 20/00; G16B 40/20; C12Q 1/6806; C12Q 1/6862; C12Q 2600/16; C12Q 2600/178

(57) ABSTRACT

The disclosure relates to data processing methods, computer readable hardware storage devices, and systems for correlating data corresponding to levels of biomarkers with various breast diseases.

### TECHNICAL FIELD

The disclosure relates to data processing methods, computer readable hardware storage devices, and systems for correlating data corresponding to levels of biomarkers with various breast diseases.

### BACKGROUND

A classifier maps input data to a category, by determining the probability that the input data classifies with a first category as opposed to another category. There are various types of classifiers, including linear discriminant classifiers, logistic regression classifiers, support vector machine classifiers, nearest neighbor classifiers, ensemble classifiers, and so forth.

### SUMMARY

The present disclosure relates to a computer-implemented method for processing data in one or more data processing devices to determine the likelihood score for, or the probability of, breast diseases (e.g. benign breast diseases or breast cancer).

In one aspect, the disclosure relates to a computer-implemented method for processing data in one or more data processing devices to determine a likelihood score that a subject has breast cancer. The method involves: inputting, into a classifier, data representing one or more values for a classifier parameter, wherein the one or more values represent expression levels of a set of non-coding RNAs in a biological sample collected from a subject, with the classifier being for determining a score indicating whether the expression levels align more closely with (A) expression levels of the same set of non-coding RNAs in biological samples collected from a first group of individuals, each of whom has breast cancer, or with (B) expression levels of the same set of non-coding RNAs in biological samples collected from a second group of individuals, none of whom has breast cancer; and determining, by the one or more data processing devices, based on application of the classifier, the likelihood score that the test subject has breast cancer.

In some embodiments, the method further involves: binding, by the one or more data processing devices, to the classifier parameter the one or more values as specified by the input data; and applying, by the one or more data processing devices, the classifier to bound values for the classifier parameter.

In some embodiments, the method further involves outputting, by the one or more data processing devices, information indicative of the likelihood that the test subject has breast cancer.

In some embodiments, the set of non-coding RNAs comprises 1, 2, 3, 4, 5, 6, 7, 8, 9 or more non-coding RNAs selected from Table 1. In some embodiments, the set of non-coding RNAs comprises 10 non-coding RNAs from Table 1. In some embodiments, the set of non-coding RNAs further comprises one or more non-coding RNAs from Table 2. In some embodiments, the set of non-coding RNAs further comprises one or more non-coding RNAs from Table 3. In some embodiments, the set of non-coding RNAs further comprises one or more non-coding RNAs from Table 4. In some embodiments, the set of non-coding RNAs further comprises one or more non-coding RNAs from Table 5. In some embodiments, the set of non-coding RNAs further comprises one or more non-coding RNAs from Table 6. In some embodiments, the set of non-coding RNAs further comprises one or more non-coding RNAs from Table 7. In some embodiments, the set of non-coding RNAs further comprises one or more non-coding RNAs from Table 8. In some embodiments, the set of non-coding RNAs further comprises one or more non-coding RNAs from Table 9. In some embodiments, the set of non-coding RNAs further comprises one or more non-coding RNAs from Table 10. In some embodiments, the set of non-coding RNAs further comprises one or more non-coding RNAs from Table 11. In some embodiments, the set of non-coding RNAs further comprises one or more non-coding RNAs from Table 12. In some embodiments, the set of non-coding RNAs further comprises one or more non-coding RNAs from Table 13. In some embodiments, the set of non-coding RNAs further comprises one or more non-coding RNAs from Table 14.

In some embodiments, each individual of the second group either (1) is a healthy individual or (2) has non-malignant breast disease or a cancer that is not breast cancer.

In some embodiments, each individual of the second group has non-malignant breast disease.

In some embodiments, each individual of the second group has a breast disease that is independently selected from the group consisting of mastitis, fat necrosis, breast cyst, papillary apocrine changes, epithelial-related calcifications, mild epithelial hyperplasia, mammary duct ectasia, periductal ectasia, non-sclerosing adenosis, periductal fibrosis, ductal hyperplasia, sclerosing adenosis, radial scar, intraductal papilloma, intraductal papillomatosis, atypical ductal hyperplasia, lobular hyperplasia, fibroadenoma, cystosarcoma phyllodes, lactating adenoma, and tubular adenoma.

In some embodiments, the classifier is a support vector machine (SVM) classifier. In some embodiments, the SVM classifier has a linear kernel with L1 penalty for regularization.

In some embodiments, the classifier comprises two or more first level sub-classifiers and one or more second level sub-classifiers. In some embodiments, the two or more first level sub-classifiers determine whether the expression levels of the set of non-coding RNAs in the biological sample collected from the subject align more closely with (A) or with (B), thereby outputting a result for each first level sub-classifier, and the one or more second level sub-classifiers combine results from the first level sub-classifiers, thereby determining one or more likelihood scores representing the likelihood that the subject has breast cancer.

In some embodiments, the two or more first level sub-classifiers are independently selected from: random forest, logistic regression, extra tree classifier, SVM, K-nearest neighbors, multilayer perceptron (MLP), deep learning classifier (DL), neural networks, feed forward neural networks, convolutional neural networks, recurrent neural networks, and gradient boosting decision trees.

In some embodiments, the one or more second level sub-classifiers are independently selected from logistic regression, gradient boosting decision trees, MLP, deep learning classifier, neural networks, feed forward neural networks, convolutional neural networks, recurrent neural networks, and SVM.

In some embodiments, the classifier comprises two or more second level sub-classifiers and a third level sub-classifier. In some embodiments, the third level sub-classifier combines the one or more likelihood scores determined by the second level sub-classifiers. In some embodiments, the third level sub-classifier is gradient boosting decision trees.

In some embodiments, the biological sample is blood, plasma, serum, saliva, urine, cerebrospinal fluid, intraductal fluid, nipple discharge, a tissue specimen, or breast milk.

In some embodiments, the expression level of each non-coding RNA is determined by amplification, sequencing, microarray analysis, multiplex assay analysis, or a combination thereof.

In some embodiments, the expression level of each non-coding RNA is determined by an amplification technique selected from the group consisting of ligase chain reaction (LCR), polymerase chain reaction (PCR), reverse transcriptase PCR, quantitative PCR, real time PCR, isothermal amplification, and multiplex PCR.

In some embodiments, the expression level of each non-coding RNA is determined by a sequencing technique selected from the group consisting of dideoxy sequencing, reverse-termination sequencing, next generation sequencing, barcode sequencing, paired-end sequencing, pyrosequencing, deep sequencing, sequencing-by-synthesis, sequencing-by-hybridization, sequencing-by-ligation, single-molecule sequencing, and single molecule real-time sequencing-by-synthesis.

In one aspect, the disclosure relates to a method of treatment. The method involves:

a) determining, or having determined, expression levels of a set of non-coding RNAs in a biological sample obtained from a subject, wherein the set of non-coding RNAs comprises five or more non-coding RNAs selected from Table 1;

b) determining, or having determined, that the expression levels align more closely with (A) expression levels of the same set of non-coding RNAs in biological samples collected from a first group of individuals who have breast cancer, than with (B) expression levels of the same set of non-coding RNAs in biological samples collected from a second group of individuals who do not have breast cancer;

c) concluding that the subject has breast cancer; and

d) administering a treatment for breast cancer to the subject.

In some embodiments, the treatment for breast cancer comprises one or more of: surgery, radiation therapy, chemotherapy, immunotherapy and cell-based therapy.

In some embodiments, the conclusion that the subject has breast cancer is corroborated by one or more further diagnostic tests, prior to administering the treatment.

In one aspect, the disclosure relates to one or more machine-readable hardware storage devices for processing data to determine a likelihood score that a subject has breast cancer, by storing instructions that are executable by one or more data processing devices to perform operations comprising:

inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals who have breast cancer, or with (B) expression levels of the set of non-coding RNAs in biological samples collected from a second group of individuals who do not have breast cancer; and

determining, by the one or more data processing devices based on application of the classifier, the likelihood score that the subject has breast cancer.

In some embodiments, the set of non-coding RNAs comprises five or more non-coding RNAs selected from Table 1.

In some embodiments, the operations further comprise:

binding, by the one or more data processing devices, to the classifier parameter the one or more values as specified by the input data; and

applying, by the one or more data processing devices, the classifier to bound values for the classifier parameter.

In some embodiments, the operations further comprise outputting, by the one or more data processing devices, information indicative of the likelihood that the test subject has breast cancer.

In one aspect, the disclosure relates to a system comprising:

one or more data processing devices; and

one or more machine-readable hardware storage devices for processing data to determine a likelihood score that a subject has breast cancer, by storing instructions that are executable by the one or more data processing devices to perform operations comprising:

inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals, each of whom has breast cancer, or with (B) expression levels of the same set of non-coding RNAs in biological samples collected from a second group of individuals, none of whom has breast cancer; and

determining, by the one or more data processing devices based on application of the classifier, the likelihood score that the subject has breast cancer.

In some embodiments, the set of non-coding RNAs comprises five or more non-coding RNAs selected from Table 1.

In some embodiments, the operations further comprise:

binding, by the one or more data processing devices, to the classifier parameter the one or more values as specified by the input data; and

applying, by the one or more data processing devices, the classifier to bound values for the classifier parameter.

In some embodiments, the operations further comprise outputting, by the one or more data processing devices, information indicative of the likelihood that the subject has breast cancer.

In one aspect, the disclosure relates to a computer-implemented method for processing data in one or more data processing devices to determine a likelihood score that a subject has a benign breast disease, the method comprising:

inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals, each of whom has a benign breast disease and does not have breast cancer, or with (B) expression levels of the same set of non-coding RNAs in biological samples collected from a second group of individuals, each of whom has breast cancer; and

determining, by the one or more data processing devices, based on application of the classifier, a likelihood score that the subject has a benign breast disease and does not have breast cancer.

In some embodiments, the set of non-coding RNAs comprises five or more non-coding RNAs selected from Table 1.

In some embodiments, the method further comprises:

binding, by the one or more data processing devices, to the classifier parameter the one or more values as specified by the input data; and

applying, by the one or more data processing devices, the classifier to bound values for the classifier parameter.

In some embodiments, the method further comprises outputting, by the one or more data processing devices, information indicative of the likelihood that the subject has a benign breast disease and does not have breast cancer.

As used herein, “a set of” refers to two or more, for example, 2, 3, 4, 5, 6, 7, 8, 9, 10, or more.

As used herein, a “blood sample” or “sample of blood” refers to whole blood, serum-reduced whole blood, lysed blood (erythrocyte-depleted blood), centrifuged lysed blood (serum-depleted, erythrocyte-depleted blood), serum-depleted whole blood or peripheral blood leukocytes (PBLs), globin-reduced RNA from blood, serum, plasma, or any other fraction of blood as would be understood by a person skilled in the art.

As used herein, “non-coding RNA” or “ncRNA” refers to an RNA molecule that is not of a type that is translated into a protein. The DNA sequence from which a functional non-coding RNA is transcribed is often called an RNA gene. Non-coding RNAs include e.g., transfer RNAs (tRNAs), ribosomal RNAs (rRNAs), microRNAs (miRNAs), siRNAs, piRNAs, snoRNAs, snRNAs, exRNAs, scaRNAs, long ncRNAs such as Xist and HOTAIR, etc.

As used herein, a “microRNA” or “miRNA” refers to a small non-coding RNA molecule that functions in RNA silencing and post-transcriptional regulation of gene expression.

As used herein, “level” or “level of expression,” when referring to RNA (e.g., non-coding RNA), means a measurable quantity (either absolute or relative quantity) of a given RNA. The quantity can be determined by various means, for example, by microarray, quantitative polymerase chain reaction (qPCR), or sequencing.

As used herein, “cancer” refers to cells having the capacity for autonomous growth within an animal. Examples of such cells include cells having an abnormal state or condition characterized by rapidly proliferating cell growth. Cancer further includes cancerous growths, e.g., tumors, oncogenic processes, metastatic tissues, and malignantly transformed cells, tissues, or organs, irrespective of histopathologic type or stage of invasiveness. Cancer further includes malignancies of the various organ systems, such as skin, respiratory, cardiovascular, renal, reproductive, hematological, neurological, hepatic, gastrointestinal, and endocrine systems; as well as adenocarcinomas, which include malignancies such as most colon cancers, breast cancer, renal-cell carcinoma, prostate cancer, testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a subject to a carcinogen(s), cancer resulting from insertion of a transgenic oncogene or knockout of a tumor suppressor gene, and cancer caused by infections, e.g., viral infections. Cancers include e.g., cancers of the skin (e.g., melanoma, unresectable melanoma, or metastatic melanoma), stomach, colon, rectum, mouth/pharynx, esophagus, larynx, liver, pancreas, lung, breast, cervix uteri, corpus uteri, ovary, prostate, testis, bladder, bone, kidney, head, neck, brain/central nervous system, and throat etc., and also Hodgkin's disease, nonHodgkin's lymphoma, sarcomas, choriocarcinoma, lymphoma, neuroblastoma (e.g., pediatric neuroblastoma), chronic lymphocytic leukemia, and non-small cell lung cancer, among others.

As used herein, a “biomarker” refers to a measurable indicator of some biological state or condition, for example, a particular RNA (e.g., non-coding RNA, miRNA, mRNA) or protein, or a particular combination of RNAs or proteins.

As used herein, the term “data” in relation to biomarkers generally refers to data reflective of the absolute and/or relative abundance (level) of a biomarker in a sample, for example, the level of one or more particular miRNAs. As used herein, a “dataset” in relation to biomarkers refers to a set of data representing the absolute and/or relative abundance (level) of one biomarker or a panel of two or more biomarkers.

As used herein, a “mathematical model” refers to a description of a system using mathematical concepts and language. The process of developing a mathematical model is termed mathematical modeling or model construction.

As used herein, the term “classifier” refers to a mathematical model with appropriate parameters that can determine a likelihood score or a probability that a test subject classifies with a first group of subjects (e.g., a group of subjects that have breast cancer) as opposed to another group of subjects (e.g., a group of subjects that do not have breast cancer).

As used herein, “random selection” or “randomly selected” refers to a method of selecting items (often called units) from a group of items or a population randomly. The probability of choosing a specific type of item from a mixed population is the proportion of that type of item in the population. For example, the probability of randomly selecting one particular gene out of a group of 10 genes is 0.1.

As herein, the terms “subject” and “patient” are used interchangeably throughout the specification and describe an animal, human or non-human, to whom the methods of the present invention can be provided. Human patients can be adult humans or juvenile humans (e.g., humans below the age of 18 years old). In some embodiments, the subject is a female. In some embodiments, the subject is over 30, 40, 50, 55, 60, 65, 70, or 75 years old.

Unless otherwise defined, technical and scientific terms used herein have the same meaning as commonly understood by one of ordinary skill in the art. Techniques and materials suitable for use in the practice or testing of the disclosed methods and systems are described below, though other techniques and materials similar or equivalent to those described can be used. The materials, methods, and examples provided here are illustrative only and not intended to be limiting. All publications, patent applications, patents, and other references mentioned herein are incorporated by reference in their entirety. In case of conflict, the present specification, including definitions, will control.

Other features and advantages of the methods and systems described herein will be apparent from the following detailed description, and from the claims.

### BRIEF DESCRIPTION OF DRAWINGS

FIG. 1 is a schematic diagram showing a system for processing and classifying data to determine a likelihood score for breast diseases.

FIG. 2 is a flow diagram of a process for processing and classifying data to determine a likelihood score for breast diseases.

FIG. 3 is a schematic diagram showing the data modeling and machine learning workflow for predicting different status (e.g., individuals with breast cancer, individuals with a benign breast disease, or healthy individuals).

FIG. 4 is a schematic diagram showing an exemplary workflow for cross-validation.

FIG. 5 is a schematic diagram showing an exemplary ensemble classification method.

FIG. 6 is a schematic diagram showing the overall architecture of an exemplary stacked ensemble model.

FIG. 7 is a confusion matrix generated by a classifier developed using the 2004 miRNA biomarker set (including all miRNAs that are listed in Tables 1-14), and using the results from the test set of 848 subjects. BRC: Breast Cancer, VOL: Volunteers, PR: Prostate Cancer, BB: Benign Breast Disease.

FIG. 8 is a confusion matrix generated by a classifier developed using the 1000 miRNA biomarker set (including all miRNAs that are listed in Tables 1-13), and using the results from the test set of 848 subjects. BRC: Breast Cancer, VOL: Volunteers, PR: Prostate Cancer, BB: Benign Breast Disease.

FIG. 9 is a confusion matrix generated by a classifier developed using the 800 miRNA biomarker set (including all miRNAs that are listed in Tables 1-12), and using the results from the test set of 848 subjects. BRC: Breast Cancer, VOL: Volunteers, PR: Prostate Cancer, BB: Benign Breast Disease.

FIG. 10 is a confusion matrix generated by a classifier developed using the 600 miRNA biomarker set (including all miRNAs that are listed in Tables 1-11), and using the results from the test set of 848 subjects. BRC: Breast Cancer, VOL: Volunteers, PR: Prostate Cancer, BB: Benign Breast Disease.

FIG. 11 is a confusion matrix generated by a classifier developed using the 500 miRNA biomarker set (including all miRNAs that are listed in Tables 1-10), and using the results from the test set of 848 subjects. BRC: Breast Cancer, VOL: Volunteers, PR: Prostate Cancer, BB: Benign Breast Disease.

FIG. 12 is a confusion matrix generated by a classifier developed using the 400 miRNA biomarker set (including all miRNAs that are listed in Tables 1-9), and using the results from the test set of 848 subjects. BRC: Breast Cancer, VOL: Volunteers, PR: Prostate Cancer, BB: Benign Breast Disease.

FIG. 13 is a confusion matrix generated by a classifier developed using the 300 miRNA biomarker set (including all miRNAs that are listed in Tables 1-8), and using the results from the test set of 848 subjects. BRC: Breast Cancer, VOL: Volunteers, PR: Prostate Cancer, BB: Benign Breast Disease.

FIG. 14 is a confusion matrix generated by a classifier developed using the 250 miRNA biomarker set (including all miRNAs that are listed in Tables 1-7), and using the results from the test set of 848 subjects. BRC: Breast Cancer, VOL: Volunteers, PR: Prostate Cancer, BB: Benign Breast Disease.

FIG. 15 is a confusion matrix generated by a classifier developed using the 200 miRNA biomarker set (including all miRNAs that are listed in Tables 1-6), and using the results from the test set of 848 subjects. BRC: Breast Cancer, VOL: Volunteers, PR: Prostate Cancer, BB: Benign Breast Disease.

FIG. 16 is a confusion matrix generated by a classifier developed using the 150 miRNA biomarker set (including all miRNAs that are listed in Tables 1-5), and using the results from the test set of 848 subjects. BRC: Breast Cancer, VOL: Volunteers, PR: Prostate Cancer, BB: Benign Breast Disease.

FIG. 17 is a confusion matrix generated by a classifier developed using the 100 miRNA biomarker set (including all miRNAs that are listed in Tables 1-4), and using the results from the test set of 848 subjects. BRC: Breast Cancer, VOL: Volunteers, PR: Prostate Cancer, BB: Benign Breast Disease.

FIG. 18 is a confusion matrix generated by a classifier developed using the 50 miRNA biomarker set (including all miRNAs that are listed in Tables 1-3), and using the results from the test set of 848 subjects. BRC: Breast Cancer, VOL: Volunteers, PR: Prostate Cancer, BB: Benign Breast Disease.

FIG. 19 is a confusion matrix generated by a classifier developed using the 25 miRNA biomarker set (including all miRNAs that are listed in Tables 1-2), and using the results from the test set of 848 subjects. BRC: Breast Cancer, VOL: Volunteers, PR: Prostate Cancer, BB: Benign Breast Disease.

FIG. 20 is a confusion matrix generated by a classifier developed using the 10 miRNA biomarker set (including all miRNAs that are listed in Table 1), and using the results from the test set of 848 subjects. BRC: Breast Cancer, VOL: Volunteers, PR: Prostate Cancer, BB: Benign Breast Disease.

FIG. 21 is a graph showing the sensitivity and specificity of classifiers developed using different biomarker sets in predicting that individuals are healthy.

FIG. 22 is a graph showing the sensitivity and specificity of classifiers developed using different biomarker sets in predicting that individuals have breast cancer.

FIG. 23 is a graph showing the sensitivity and specificity of classifiers developed using different biomarker sets in predicting that individuals have a benign breast disease.

### DETAILED DESCRIPTION

This disclosure relates to, e.g., a computer-implemented method for processing data to determine a likelihood score for breast diseases. A data processing system consistent with this disclosure applies classifiers to data corresponding to levels of a set of biomarkers (e.g., non-coding RNAs) in a biological sample collected from a subject.

The practice of the present disclosure will also partly employ, unless otherwise indicated, techniques of molecular biology, microbiology and recombinant DNA that are familiar to those skilled in the art.

Data Processing System

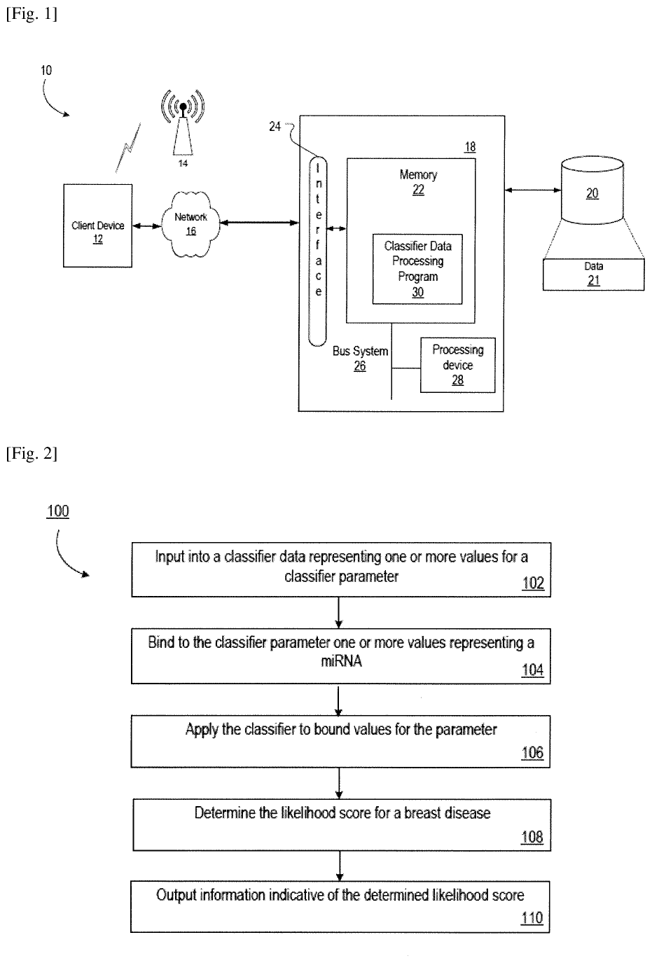

Referring to FIG. 1, system 10 classifies groups of data via binding data to parameters and applying a classifier to the input data, and outputs information indicative of a likelihood score for a particular status (e.g., breast disease or a benign breast disease). System 10 includes client device 12, data processing system 18, and data repository 20. Data processing system 18 receives data from, for example, client device 12 via network 16, and/or wireless device 14. System 10 may include wireless device 14 and/or network 16.

Data processing system 18 retrieves, from data repository 20, data 21 representing one or more values for a classifier parameter that represents level of biomarkers (e.g., non-coding RNAs or miRNA) from a set of biomarkers in a sample from a test subject, as described in further detail below. Data processing system 18 inputs the retrieved data into a classifier, e.g., into classifier data processing program 30. In this embodiment, classifier data processing program 30 is programmed to execute a data classifier. There are various types of data classifiers, including, e.g., linear discriminant classifiers, support vector machine classifiers, nearest neighbor classifiers, ensemble classifiers, random forest classifiers, logistic regression classifiers, extra trees classifiers, gradient boosting classifiers, multi-layer perceptron classifiers (e.g., MLP 3 Classifier, and MLP 4 Classifier), deep learning classifiers, neural networks, feed forward neural networks, convolutional neural networks, recurrent neural networks, and so forth.

In some embodiments, the classifier data processing program 30 can be configured to execute a classifier as described herein. The data processing program 30 can output a likelihood score indicating the probability that the set of biomarker levels classifies with (A) levels of the same set of biomarkers (e.g., non-coding RNAs) in biological samples collected from a first group of individuals, each of whom has a particular status (e.g., breast cancer), or with (B) levels of the same set of biomarkers (e.g., non-coding RNAs) in biological samples collected from a second group of individuals, none of whom has the status (e.g., breast cancer).

In some embodiments, the biomarkers can be RNA (e.g., non-coding RNAs in general, miRNA, siRNAs, piRNAs, snoRNAs, snRNAs, exRNAs, scaRNAs or long ncRNAs, etc.).

In some embodiments, data processing system 18 binds to classifier parameter one or more values representing levels of a set of biomarkers, as specified in retrieved data 21. Data processing system 18 binds values of the data to the classifier parameter by modifying a database record such that a value of the parameter is set to be the value of data 21 (or a portion thereof). Data 21 includes a plurality of data records that each have one or more values for the parameter. In some embodiments, data processing system 18 applies classifier data processing program 30 to each of the records by applying classifier data processing program 30 to the bound values for the parameter. Based on application of classifier data processing program 30 to the bound values (e.g., as specified in data 21 or in records in data 21), data processing system 18 determines a likelihood score indicating a probability that the set of biomarker levels from the test subject classifies with the set of biomarker levels for a particular group of subjects, as opposed to the set of biomarker levels for some other group or groups of subjects, and outputs, e.g., to client device 12 via network 16 and/or wireless device 14, data indicative of the determined likelihood score for the status (e.g., breast cancer or a benign breast disease) for the test subject.

Data processing system 18 generates data for a graphical user interface that, when rendered on a display device of client device 12, display a visual representation of the output.

In some embodiments, data processing system 18 generates the classifier by applying the mathematical model to a dataset to determine parameters of a classifier (e.g., parameters for linear discriminant classifiers, support vector machine classifiers, nearest neighbor classifiers, ensemble classifiers, random forest classifiers, logistic regression classifiers, extra trees classifiers, gradient boosting classifiers, multi-layer perceptron classifier, deep learning classifier, neural networks, feed forward neural networks, convolutional neural networks, or recurrent neural networks). The values for these parameters can be stored in data repository 20 or memory 22.

Client device 12 can be any sort of computing device capable of taking input from a user and communicating over network 16 with data processing system 18 and/or with other client devices. Client device 12 can be a mobile device, a desktop computer, a laptop computer, a cell phone, a personal digital assistant (PDA), a server, an embedded computing system, and so forth.

Data processing system 18 can be any of a variety of computing devices capable of receiving data and running one or more services. In some embodiments, data processing system 18 can include a server, a distributed computing system, a desktop computer, a laptop computer, a cell phone, and the like. Data processing system 18 can be a single server or a group of servers that are at a same position or at different positions (i.e., locations). Data processing system 18 and client device 12 can run programs having a client-server relationship to each other. Although distinct modules are shown in the figure, in some embodiments, client and server programs can run on the same device.

Data processing system 18 can receive data from wireless device 14 and/or client device 12 through input/output (I/O) interface 24 and data repository 20. Data repository 20 can store a variety of data values for classifier data processing program 30. The classifier data processing program (which may also be referred to as a program, software, a software application, a script, or code) can be written in any form of programming language, including compiled or interpreted languages, or declarative or procedural languages, and it can be deployed in any form, including as a stand-alone program or as a module, component, subroutine, or other unit suitable for use in a computing environment. The classifier data processing program may, but need not, correspond to a file in a file system. The program can be stored in a portion of a file that holds other programs or information (e.g., one or more scripts stored in a markup language document), in a single file dedicated to the program in question, or in multiple coordinated files (e.g., files that store one or more modules, sub programs, or portions of code). The classifier data processing program can be deployed to be executed on one computer or on multiple computers that are located at one site or distributed across multiple sites and interconnected by a communication network.

In some embodiments, data repository 20 stores data 21 indicative of the levels of biomarkers (e.g., non-coding RNA, such as miRNA), for example, the levels of non-coding RNA for a group of individuals who have a breast disease (e.g., breast cancer), a group of individuals who are healthy (e.g., do not have the breast disease), and/or a test subject. In another embodiment, data repository 20 stores parameters of a classifier. Interface 24 can be a type of interface capable of receiving data over a network, including, e.g., an Ethernet interface, a wireless networking interface, a fiberoptic networking interface, a modem, and so forth. Data processing system 18 also includes a processing device 28. As used herein, a “processing device” encompasses all kinds of apparatuses, devices, and machines for processing information, such as a programmable processor, a computer, or multiple processors or computers. The apparatus can include special purpose logic circuitry, e.g., an FPGA (field programmable gate array) or an ASIC (application specific integrated circuit) or RISC (reduced instruction set circuit). The apparatus can also include, in addition to hardware, code that creates an execution environment for the computer program in question, e.g., code that constitutes processor firmware, a protocol stack, an information base management system, an operating system, or a combination of one or more of them.

Data processing system 18 also includes a memory 22 and a bus system 26, including, for example, a data bus and a motherboard, which can be used to establish and to control data communication between the components of data processing system 18. Processing device 28 can include one or more microprocessors. Generally, processing device 28 can include an appropriate processor and/or logic that is capable of receiving and storing data, and of communicating over a network. Memory 22 can include a hard drive and a random access memory storage device, including, e.g., a dynamic random access memory, or other types of non-transitory, machine-readable storage devices. Memory 22 stores classifier data processing program 30 that is executable by processing device 28. These computer programs may include a data engine (not shown) for implementing the operations and/or the techniques described herein. The data engine can be implemented in software running on a computer device, hardware or a combination of software and hardware.

Referring to FIG. 2, data processing system 18 performs process 100 to output a likelihood score indicative of the probability for a breast disease. In operation, data processing system 18 inputs (102), into a classifier, data representing one or more values for a classifier parameter. The data can come from wireless devices 14, client device 12, and/or data repository 20. Data processing system 18 binds (104) one or more values representing levels of biomarkers (e.g., miRNA) to the classifier parameter. Data processing system 18 applies (106) the classifier to bound values for the parameter, and determines (108) a likelihood score indicating a probability of a breast disease. Data processing system 18 outputs (110), by the one or more data processing devices 28, information (e.g., likelihood score) indicative of probability of a breast disease. The output may be transmitted to a display device, e.g., a CRT (cathode ray tube) or LCD (liquid crystal display) monitor, or transmitted to client device 12, or wireless device 14 through network 16.

Breast Diseases

This disclosure provides methods for determining whether a subject is likely to have a breast disease, particularly breast cancer. In some embodiments, the subject has been identified as likely having some sort of breast disease, e.g., because the subject has experienced some discomfort or physical changes in the breast area, but the exact reason of the discomfort or change has not been determined. There is a need to determine whether the subject has a life-threatening disease, e.g., breast cancer, that will require particular treatments. In some embodiments, the subject is suspected to have breast cancer.

The methods described herein can be used to determine whether a subject has a breast disease (e.g., a neoplastic breast disease or a non-neoplastic breast disease). Neoplastic breast diseases can be benign or malignant. Non-limiting examples of benign neoplastic breast diseases include, e.g., fibroadenoma, phyllodes tumor (e.g. Cystosarcoma phyllodes or “Giant fibroadenoma”), lipoma, adenoma (e.g. lactating, tubular or nipple). Non-limiting examples of malignant neoplastic breast diseases include ductal carcinoma, lobular carcinoma, medullary carcinoma, and mucinous (colloid) carcinoma. Ductal carcinoma can be in situ (DCIS) (e.g. comedocarcinoma, solid cribiform, papillary, or micropapillary), or invasive. Lobular carcinoma can be in situ or invasive/infiltrating.

In some embodiments, the breast diseases as described herein are non-neoplastic breast diseases. Non-neoplastic breast diseases can include, e.g., inflammatory breast diseases or breast diseases that are associated with or involve fibrocystic changes (FCC). Non-limiting examples of inflammatory breast diseases include mastitis (e.g. acute or granulomatous mastitis) and fat necrosis. Breast diseases that are associated with or involve fibrocystic changes can be non-proliferative, such as, but not limited to cyst (e.g. simple or complex/complicated cyst), papillary apocrine changes, epithelial-related calcifications, mild epithelial hyperplasia, mammary duct ectasia, periductal ectasia, non-sclerosing adenosis, and periductal fibrosis. Breast diseases that are associated with or involve fibrocystic changes can also be proliferative without atypia, such as ductal hyperplasia (usual type), sclerosing adenosis, radial scar, intraductal papilloma or papillomatosis. Breast diseases that are associated with or involve fibrocystic changes can also be proliferative with atypia, such as, but not limited to atypical ductal hyperplasia and lobular hyperplasia.

In some embodiments, the methods and systems described herein can be used to determine whether a subject has breast cancer. As used herein, the term “breast cancer” refers to cancer that develops from breast tissue, including e.g., ductal carcinoma, lobular carcinoma, medullary carcinoma, mucinous (colloid) carcinoma, etc. External signs of breast cancer may include e.g., a lump in the breast, a change in breast shape, dimpling of the skin, fluid coming from the nipple, a newly inverted nipple, or a red or scaly patch of skin. Thus, in some embodiments, the methods described herein can be used to determine whether a subject having one or more signs of breast cancer has breast cancer. In some cases, the methods described herein can be used in breast cancer screening, including in subjects with no signs of breast cancer. In some embodiments, the subject has been identified as being at risk of developing breast cancer. Some common risk factors for breast cancer include, but are not limited to, age (e.g., being at least 40, 45, 50, 55, 60, 65, 70, 75, or 80 years old), genetic factors, obesity, lack of exercise, high age of first childbirth (e.g., giving birth to the first child when at least 30, 35, or 40 years old), having never given birth, taking hormonal contraceptives, or experiencing pre-menopause. Some genetic factors for breast cancer include, but are not limited to, carrying harmful mutations in BRCA1 and/or BRCA2, carrying harmful mutations in genes associated with metabolism of estrogens and/or carcinogens (e.g., Cytochrome P450 (family 1, member A1), CYP1B1, CYP17A1, CYP19, Catechol-O-methyl transferase, N-acetyltransferase 2, Glutathione S-transferase Mu 1, GSTP1, GSTT, etc.), carrying harmful mutations in genes associated with estrogen, androgen and vitamin D action (e.g., ESR1, AR, VDR), carrying harmful mutations in genes associated with estrogen-induced gene transcription pathway (e.g., AIB1), and carrying harmful mutations in genes associated with estrogen-induced DNA damage response pathways (e.g., CHEK2, HRAS1, XRCC1, XRCC3, XRCC5).

The two most commonly used screening methods, physical examination of the breasts by a healthcare provider and mammography, can offer a first indication that a lump is cancer, but may also detect some other types of lesions, such as a simple cyst. In some embodiments, the methods described herein can be performed on a subject who has been identified as requiring further analysis after the initial examination.

Breast cancer sometimes can be confirmed by microscopic analysis of a sample or biopsy of the affected area of the breast. However, because these procedures are invasive, these procedures are usually performed only when the circumstances (e.g., imaging by ultrasound, MRI, mammography, or diagnosis made by the methods described herein) are sufficient to warrant excisional biopsy as the definitive diagnostic method. For example, when the methods described herein or some other methods indicate that a subject is very likely to have breast cancer, a healthcare provider can remove a sample of the fluid in the subject's breast lump for microscopic analysis (a procedure known as fine needle aspiration, or fine needle aspiration and cytology-FNAC) to help establish or confirm the diagnosis. A finding of clear fluid makes the lump highly unlikely to be cancerous, but bloody fluid may be sent off for inspection under a microscope for cancerous cells. Options for biopsy include, e.g., a core biopsy or vacuum-assisted breast biopsy, which are procedures in which a section of the breast lump is removed; or an excisional biopsy, in which the entire lump is removed.

The methods provided herein also include treating a breast disease, e.g., administering a treatment for breast cancer to a subject identified as having breast cancer, using the presently disclosed methods. Suitable treatments for a given patient's breast cancer may be determined based on particular genomic markers (e.g. presence of mutations known to be associated with breast cancer, such as in the BRCA1 or BRCA2 gene); or based on the type, subtype (e.g. hormone receptor status such as ER, PR, and HER2 status), and stage of the breast cancer; and/or based on patient age, general health, menopausal status, and a patient's preferences. Treatments for breast cancer can include, for example, surgery, chemotherapy, radiation therapy, cryotherapy, hormonal therapy, cell-based therapies (e.g. CAR-T therapy), and immune therapy. Ductal carcinoma in situ can often be treated with breast-conserving surgery and radiation therapy without further lymph node exploration or systemic therapy. Stages I and II breast cancers are usually treated with breast-conserving surgery and radiation therapy. Choice of adjuvant systemic therapy can depend on lymph node environment, hormone receptor status, ERBB2 overexpression, and patient age and menopausal status. Node-positive breast cancer is generally treated systemically with chemotherapy, endocrine therapy (for hormone receptor-positive cancer), and trastuzumab (for cancer overexpressing ERBB2). Anthracycline- and taxane-containing chemotherapeutic regimens may be used. Stage III breast cancer typically requires induction chemotherapy to downsize the tumor to facilitate breast-conserving surgery. Inflammatory breast cancer, although considered stage III, is aggressive and requires induction chemotherapy followed by mastectomy, rather than breast-conserving surgery, as well as axillary lymph node dissection and chest wall radiation. Additional treatments for breast cancer are known in the art.

In some embodiments, the disclosure further relates to treatment for some other non-cancer breast diseases (e.g. inflammatory breast diseases, breast diseases associated with or involving fibrocystic changes, or benign neoplastic breast diseases). Mastitis can be treated with steroids or ductal lavage. Treatment options for breast cysts include, e.g., fine-needle aspiration and surgical excision. Treatment options for breast pain can include over-the-counter pain relievers or oral contraceptives.

Sample Preparation

Samples for use in the techniques described herein include any of various types of biological molecules, cells and/or tissues that can be isolated and/or derived from a subject. The sample can be isolated and/or derived from any fluid, cell or tissue. In some embodiments, the sample is blood, plasma, serum, lymph, saliva, urine, cerebrospinal fluid, intraductal fluid, nipple discharge, a tissue specimen, or breast milk. The sample that is isolated and/or derived from a subject can be assayed for biomarker levels (e.g., gene expression products, miRNA levels, non-coding RNA levels).

In some embodiments, fine needle aspiration is performed to collect a sample from a lesion in the breast tissue. The specimen collected can be any fluid from the lesion, e.g., from a cyst and/or cells from the lesion.

In some embodiments, the sample is a fluid sample, a lymph sample, a lymph tissue sample, or a blood sample. In some embodiments, the sample can be one isolated and/or derived from any fluid and/or tissue that predominantly comprises blood cells. In some embodiments, the sample is isolated and/or derived from peripheral blood. In other embodiments, the sample may be isolated and/or derived from alternative sources, including from any of various types of lymphoid tissue.

In one embodiment, a sample of blood is obtained from an individual according to methods well known in the art. In some embodiments, a drop of blood is collected from a simple pin prick made in the skin of an individual. Blood may be drawn from any part of the body (e.g., a finger, hand, wrist, arm, leg, foot, ankle, abdomen, or neck) using techniques known to one of skill in the art, such as phlebotomy. Examples of samples isolated and/or derived from blood include samples of whole blood, serum-reduced whole blood, serum-depleted blood, erythrocyte-depleted blood, serum, and plasma. In some embodiments, a blood sample is be collected by venipuncture from the cubital space. A collected blood sample may be processed, e.g., by centrifugation, to isolate serum or plasma.

In some embodiments, whole blood collected from an individual is fractionated (i.e., separated into components) before measuring the absolute and/or relative abundance (level) of a biomarker in the sample. In some embodiments, the blood is allowed to clot, and the clot is removed to produce serum. In other embodiments, an anticoagulant is added to the whole blood; after removal of cells from the blood (e.g., by centrifugation), plasma results. In one embodiment, blood is serum-depleted (or serum-reduced). In other embodiments, the blood is plasma-depleted (or plasma-reduced). In yet other embodiments, blood is erythrocyte-depleted or erythrocyte-reduced. In some embodiments, erythrocyte reduction is performed by preferentially lysing the red blood cells. In other embodiments, erythrocyte depletion or reduction is performed by lysing the red blood cells and further fractionating the remaining cells. In yet other embodiments, erythrocyte depletion or reduction is performed, but the remaining cells are not further fractionated. In other embodiments, blood cells are separated from whole blood collected from an individual using other techniques known in the art.

In some embodiments, miRNAs can be isolated from exosomes present in body fluids such as blood, plasma, serum, urine, cerebrospinal fluid, breast milk, or saliva. Exosomes are small cell-derived vesicles that function in intercellular communication processes, carrying a cargo of miRNAs from one tissue to another. Once released into the extracellular fluid, exosomes fuse with other cells and transfer their cargo to the acceptor cell. Exosomes are secreted by many cell types, including, e.g., B cells, dendritic cells, T cells, platelets, and tumor cells. In some embodiments, miRNAs are purified from other types of RNAs by first isolating exosomes from a biofluid (such as blood), e.g., by differential ultracentrifugation, and then isolating the miRNAs from the isolated exosomes. Methods of isolating exosomes and miRNAs are described, e.g., in US 2019/0085410 A1; U.S. Pat. No. 8,211,653 B2; and Sanz-Rubio et al. “Stability of circulating Exosomal miRNAs in healthy subjects.” Scientific reports (2018) 8:10306.

Other methods of determining levels of miRNAs do not require any step of isolating exosomes.

RNA Quantification

The level of a biomarker (e.g., a particular non-coding RNA (e.g. a miRNA)) can be determined by any means known in the art. The quantity of a given RNA can be determined by various means, for example, by microarray (e.g., RNA microarray, cDNA microarray), quantitative polymerase chain reaction (qPCR), or sequencing technology (e.g., RNA-Seq). In the methods described herein, the level of a biomarker can be taken to represent the level of expression of a non-coding RNA (e.g., a miRNA).

In some embodiments, a level of a biomarker (when referring to RNA) is stated as a number of PCR cycles required to reach a threshold amount of a particular RNA or DNA, e.g., 10, 15, 20, 25, 30, 35, 40, 45, 50, 55, or 60 cycles. The level of a biomarker, when referring to RNA, can also refer to a measurable quantity of a given nucleic acid as determined relative to the amount of total RNA, or cDNA used in qRTPCR, in which the amount of total RNA used is, for example, 100 ng, 50 ng, 25 ng, 10 ng, 5 ng, 1.25 ng, 0.05 ng, 0.3 ng, 0.1 ng, 0.09 ng, 0.08 ng, 0.07 ng, 0.06 ng, or 0.05 ng. The level of a nucleic acid can be determined by any methods known in the art. For microarray analysis, the level of a nucleic acid is measured by hybridization analysis using nucleic acids corresponding to RNA isolated from the samples, according to methods well known in the art. The label used in the samples can be a luminescent label, an enzymatic label, a radioactive label, a chemical label or a physical label. In some embodiments, target and/or probe nucleic acids are labeled with a fluorescent molecule. The level of a biomarker, when referring to RNA, can also refer to a measurable quantity of a given nucleic acid as determined relative to the amount of total RNA or cDNA used in a microarray hybridization assay. In some embodiments, the amount of total RNA is 10 μg, 5 μg, 2.5 μg, 2 μg, 1 μg, 0.5 μg, 0.1 μg, 0.05 μg, 0.01 μg, 0.005 μg, 0.001 μg, or the like. In some embodiments, the level of an RNA biomarker is represented by the number of mapped reads identified by RNA-Seq. The reads can be further normalized, e.g., by the total number of mapped reads, so that biomarker levels are expressed as Fragments Per Kilobase of transcript per Million mapped reads (FPKM).

In some embodiments, RNA is obtained from a nucleic acid mix using a filter-based RNA isolation system. One such method is described in pp. 55-104, in RNA Methodologies, A laboratory guide for isolation and characterization, 2nd edition, 1998, Robert E. Farrell, Jr., Ed., Academic Press. In some embodiments, RNA is prepared using a well-known system for isolating RNA (including isolating total RNA or non-coding RNA (e.g., miRNA), and the like). In some embodiments, microRNA (miRNA) can be quantified using quantitative polymerase chain reaction (qPCR) (e.g., as described in Chen et al., Nucleic Acids Res., 33(20):e179, 2005; Redshaw et al., BioTechniques. 54(3):155-164, 2013; and Balcells et al., BMC Biotechnol. 11:70, 2011), microarray (e.g., as described in Sato et al., PLoS One 4(5):e5540, 2009), next-generation sequencing (e.g., as described in Chatterjee et al., Sci Rep. 5:10438, 2015), isothermal amplification (e.g., as described in Zhao et al., Chem Rev. 115(22):12491-12545, 2015), and near-infrared technology (e.g., as described in Miao et al., Anal Chem. 88(15):7567-7573, 2016). Non-limiting examples of isothermal amplification for miRNA quantification include: exponential amplification (e.g., as described in Jia et al. Angew Chem Int Ed Engl. 49(32):5498-5501, 2010), rolling circle amplification (e.g., as described in Tian et al., Nano. 7(3):987-993, 2015), duplex-specific nuclease signal amplification (e.g., as described in Yin et al., J Am Chem Soc. 134(11):5064-5067, 2012), and hybridization chain reaction (e.g., as described in Dirks et al., Proc Natl Acad Sci USA. 101(43):15275-15278, 2004).

In some embodiments, the presence of a miRNA or other non-coding RNA is detected using any of the appropriate PCR methods known in the art. Such PCR methods include, without limitation, RT-PCR, real-time PCR, semi-quantitative PCR, qPCR, and multiplex PCR. In other embodiments, a miRNA can be detected by hybridization to one or more miRNA probes which may be comprised on a microarray or a biochip or in a hybridization solution. In some preferred embodiments, a miRNA signature may be determined by miRNA microarray or multiplex hybridization and analysis. In some embodiments, the one or more miRNA probes may be attached to a solid phase sample collection medium (such as in a multiplex array or on a microarray). In some embodiments, the miRNA probe(s) may be attached to a solid phase sample collection medium made of a material such as glass, modified or functionalized glass, plastic, nylon, cellulose, nitrocellulose, resin, silica, or silica-based material. The miRNA probes may be attached to the solid phase sample collection medium covalently or non-covalently. In some embodiments, the screening of miRNA comprises amplification, sequencing, microarray analysis, multiplex array analysis, or a combination thereof. The amplification can be performed by ligase chain reaction (LCR), polymerase chain reaction (PCR), reverse-transcriptase PCR, quantitative PCR, real time PCR, isothermal amplification, and multiplex PCR. In some cases, the sequencing technique is selected from the group consisting of dideoxy sequencing, reverse-termination sequencing, next generation sequencing, barcode sequencing, paired-end sequencing, pyrosequencing, deep sequencing, sequencing-by-synthesis, sequencing-by-hybridization, sequencing-by-ligation, single-molecule sequencing, single molecule real-time sequencing-by-synthesis. Some of the screening and detection methods are described, e.g., in US 2017/0145515.

In some embodiments, the level of miRNA can be quantified using the Fireplex® particle technology (Abcam). In this assay, oligo DNA probes embedded within hydrogel particles have a miRNA binding site specific for a particular target miRNA. Flanking the miRNA binding site on the probes are universal adapter-binding regions required for subsequent amplification. When particles are mixed with sample (e.g., either crude biofluids after a digest step or purified RNA), target miRNAs present in the sample bind to their specific probes. After miRNA capture, particles are rinsed to remove unbound materials and remove potential inhibitors of subsequent steps, such as heparin. In some embodiments, a mixture of particles specific to different target miRNAs is present in a given well, so that multiple miRNAs are targeted within that well. This can greatly contribute to assay reproducibility, as all of the miRNAs quantified per well are exposed to the same conditions throughout the assay. Labeling mix containing universal adaptors (DNA) and ligation enzymes is mixed with the particles, resulting in the ligation of adaptors on either side of the target miRNA to generate a fusion DNA-RNA-DNA molecule. Particles are rinsed, and unligated adaptors, which are too short to remain on the probe through this step, are washed away. Ligated miRNAs and adaptors are eluted from the probe, and PCR is performed using primers specific for the universal adaptors. The reverse primer is labeled with biotin, allowing reporting of the target miRNAs at a later step. After amplification, samples can be mixed with particles again, to be recaptured by miRNA-specific probes. A fluorescent reporter is added that binds to the biotin incorporated during amplification. Fluorescence is then measured on the particles by flow cytometry.

Mathematical Models

A mathematical model can be used to determine, from the non-coding RNA data, the likelihood score that a subject has a disease (e.g., a breast disease, breast cancer or a benign breast disease) or that the subject is healthy.

Various types of mathematical models can be used, including, e.g., a regression model in the form of logistic regression, principal component analysis, linear discriminant analysis, correlated component analysis, random forest, logistic regression, extra tree classifier, Support Vector Machine (SVM), K-nearest neighbors, multi-layer perceptron (MLP), deep learning classifiers, neural networks, feed forward neural networks, convolutional neural networks, recurrent neural networks or gradient boosting decision trees. These models can be used in connection with data from different sets of non-coding RNAs (e.g., miRNAs). The model for a given set of non-coding RNAs is applied to a training dataset, generating relevant parameters for a classifier. In some cases, these models with relevant parameters for a classifier can be applied back to the training dataset, or applied to a validation (or test) dataset to evaluate the classifier.

To apply the classifier to a test subject, a sample is collected from the test subject. The levels of the selected biomarkers (e.g., non-coding RNAs) in the sample are determined. These data are then tested in accordance with the classifier, and the subject's likelihood score for having a disease (e.g., a breast disease, breast cancer or a benign breast disease) is calculated. The likelihood score can be used to determine the probability that the subject has breast cancer or a benign breast disease, or the probability that the subject is healthy; or a value indicative of the probability that the subject has breast cancer or a benign breast disease or indicative of the probability that the subject is healthy.

The classifier can determine whether the subject has breast cancer or a benign breast disease. Based on that determination, a physician can determine an appropriate treatment regimen for the subject, or can order further diagnostic tests (such as a biopsy) to confirm or further refine the diagnosis.

Various types of mathematical models with appropriate parameters can be used as classifiers. These mathematical models can include, e.g., the regression model in the form of logistic regression or linear regression, principal component analysis, linear discriminant analysis, correlated component analysis, support vector machine, nearest neighbor, random forest, extra trees, gradient boosting, multilayer perceptron, deep learning, neural networks (e.g., feed forward neural networks, convolutional neural networks, recurrent neural networks), etc.

These mathematical models can be used in connection with a set of biomarkers, e.g., a set of non-coding RNAs such as miRNAs. The models can then be applied to a training dataset, generating appropriate classifier parameters, thus creating a classifier. The classifier can then be used to determine the likelihood score for a breast disease (e.g., breast cancer or a benign breast disease) or the likelihood score that the subject is healthy.

Some exemplary sets of biomarkers that can be used in the classifiers are listed in the tables below. These biomarkers and the sequences thereof are described in the miRBase database (release 20). The miRBase database is a searchable database of published miRNA sequences and annotation. Each entry in the miRBase database represents a predicted hairpin portion of a miRNA transcript (termed mir in the database), with information on the location and sequence of the mature miRNA sequence (termed miR) and a unique name assigned to each (e.g., “hsa-miR-495-3p”). Both hairpin and mature sequences are available for searching and browsing, and entries can also be retrieved by name, keyword, references and annotation. The miRBase database is described in Griffiths-Jones et al. “miRBase: microRNA sequences, targets and gene nomenclature.” Nucleic acids research 34.suppl_1 (2006): D140-D144. The annotation of miRNA is described in, e.g., Ambros et al. “A uniform system for microRNA annotation.” RNA 9.3 (2003): 277-279.

(1)

Core classifiers developed using 1-10 markers

(“core set” or “Set 1”)

A core classifier is a classifier that uses the levels of a core set of non-coding RNAs as input. The core set of non-coding RNAs can include, e.g., about or at least 1, 2, 3, 4, 5, 6, 7, 8, 9, or 10 non-coding RNAs from Table 1, which (like the rest of the tables below) identifies each non-coding RNA (all of which are miRNAs) by its official miRBase database name. Various types of mathematical models with appropriate parameters can be used in connection with this core set of non-coding RNAs. In some embodiments, the core set of non-coding RNAs comprises three or more, four or more, or five or more non-coding RNAs selected from Table 1. Where multiple miRNA names are shown in one box, they represent alternative names of the same miRNA. The prefix “hsa” in the names stands for Homo sapiens.

TABLE 1
hsa-miR-495-3p
hsa-miR-1976
hsa-miR-1973
hsa-miR-1972
hsa-miR-3065-3p
hsa-miR-4778-3p
hsa-miR-1914-3p
hsa-miR-3689a-5p,
hsa-miR-3689b-5p,
or hsa-miR-3689e
hsa-miR-4999-3p
hsa-miR-6873-3p

(2) Classifiers Developed Using Set 2 Markers

Also provided herein is a set of non-coding RNAs (Set 2) that can be used in classifiers. Set 2 of non-coding RNAs includes, in addition to one or more (up to all) of the core set of non-coding RNAs from Table 1, about or at least 1, 2, 3, 4, 5, 6, 7, 8, 9, 10, 11, 12, 13, 14, or all 15 non-coding RNAs selected from Table 2—i.e., up to as many as 25 miRNAs in total. Various types of mathematical models with appropriate parameters can be used in connection with this set of non-coding RNAs.

TABLE 2
hsa-miR-6841-5p
hsa-miR-552-5p
hsa-miR-6817-5p
hsa-miR-4633-5p
hsa-miR-4704-3p
hsa-miR-671-3p
hsa-miR-514a-3p
hsa-miR-6864-5p
hsa-miR-4529-3p
hsa-miR-216b-3p
hsa-miR-509-3p
hsa-miR-526b-3p
hsa-miR-29b-1-5p
hsa-miR-5587-5p
hsa-miR-216a-3p

(3)

Classifiers Developed Using Set 3 Markers

Also provided herein is a set of non-coding RNAs (Set 3) that includes some or all non-coding RNAs of Set 2 and about or at least 1, 2, 3, 4, 5, 6, 7, 8, 9, 10, 11, 12, 13, 14, 15, 16, 17, 18, 19, 20, 21, 22, 23, 24, or 25 non-coding RNAs selected from Table 3—i.e., up to as many as 50 miRNAs in total. Classifiers developed using the Set 3 markers are provided herein. Various types of mathematical models with appropriate parameters can be used in connection with this set of non-coding RNAs.

TABLE 3
hsa-miR-33b-3p
hsa-miR-4773
hsa-miR-302c-3p
hsa-miR-4477b
hsa-miR-4477a
hsa-miR-96-3p
hsa-miR-4715-3p
hsa-miR-5004-3p
hsa-miR-4482-5p
hsa-miR-1273g-5p
hsa-miR-151b
hsa-miR-4496
hsa-miR-4495
hsa-miR-4494
hsa-miR-4493
hsa-miR-4491
hsa-miR-4490
hsa-miR-514b-5p
hsa-miR-1251-3p
hsa-miR-4499
hsa-miR-4498
hsa-miR-4654
hsa-miR-4657
hsa-miR-5708
hsa-miR-6795-3p

(4)

Classifiers Developed Using Set 4 Markers

Also provided herein is a set of non-coding RNAs (Set 4) that includes some or all non-coding RNAs of Set 3 and about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 4—i.e., up to as many as 100 miRNAs in total. Classifiers developed using the Set 4 markers are provided herein. Various types of mathematical models with appropriate parameters can be used in connection with this set of non-coding RNAs.

TABLE 4
hsa-miR-205-3p
hsa-miR-5704
hsa-miR-5705
hsa-miR-5706
hsa-miR-5707
hsa-miR-5700
hsa-miR-4658
hsa-miR-5702
hsa-miR-5703
hsa-miR-4708-3p
hsa-miR-6815-5p
hsa-miR-4290
hsa-miR-4527
hsa-miR-8077
hsa-miR-8076
hsa-miR-6789-3p
hsa-miR-6773-5p
hsa-miR-4796-3p
hsa-miR-3678-3p
hsa-miR-630
hsa-miR-631
hsa-miR-632
hsa-miR-633
hsa-miR-635
hsa-miR-6508-3p
hsa-miR-637
hsa-miR-639
hsa-miR-6762-3p
hsa-miR-5588-5p
hsa-miR-139-3p
hsa-miR-6764-5p
hsa-miR-433-3p
hsa-miR-455-5p
hsa-miR-6882-3p
hsa-miR-3141
hsa-miR-3142
hsa-miR-2682-5p
hsa-miR-7853-5p
hsa-miR-3136-3p
hsa-miR-3147
hsa-miR-3148
hsa-miR-3149
hsa-miR-208b-5p
hsa-miR-6878-5p
hsa-miR-3617-5p
hsa-miR-519e-5p
hsa-miR-6831-5p
hsa-miR-4746-5p
hsa-miR-6739-5p
hsa-miR-6886-3p

(5)

Classifiers Developed Using Set 5 Markers

Also provided herein is a set of non-coding RNAs (Set 5) that includes some or all non-coding RNAs of Set 4 and about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 5—i.e., up to as many as 150 miRNAs in total. Classifiers developed using the Set 5 markers are provided herein. Various types of mathematical models with appropriate parameters can be used in connection with this set of non-coding RNAs.

TABLE 5
hsa-miR-3925-3p
hsa-miR-708-5p
hsa-miR-2467-3p
hsa-miR-214-5p
hsa-miR-215-5p
hsa-miR-548aa, or
hsa-miR-548t-3p
hsa-miR-6822-3p
hsa-miR-3124-3p
hsa-miR-3158-5p
hsa-miR-515-5p
hsa-miR-6821-3p
hsa-miR-6834-3p
hsa-miR-627-3p
hsa-miR-3923
hsa-miR-3920
hsa-miR-3921
hsa-miR-3926
hsa-miR-3924
hsa-miR-4251
hsa-miR-6761-3p
hsa-miR-4253
hsa-miR-3929
hsa-miR-3156-3p
hsa-miR-4254
hsa-miR-4256
hsa-miR-16-l-3p
hsa-miR-4735-5p
hsa-miR-4757-3p
hsa-miR-3187-5p
hsa-miR-539-3p
hsa-miR-4655-3p
hsa-miR-1285-5p
hsa-miR-3145-5p
hsa-miR-125a-5p
hsa-miR-3144-5p
hsa-miR-4700-3p
hsa-miR-7856-5p
hsa-miR-1178-3p
hsa-miR-593-3p
hsa-miR-204-5p
hsa-miR-6866-5p
hsa-miR-7-2-3p
hsa-miR-6867-3p
hsa-miR-544a
hsa-miR-338-3p
hsa-miR-6826-3p
hsa-miR-4786-5p
hsa-miR-4753-3p
hsa-miR-548ar-3p
hsa-miR-7-l-3p

(6)

Classifiers Developed Using Set 6 Markers

Also provided herein is a set of non-coding RNAs (Set 6) that includes some or all non-coding RNAs of Set 5 and about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 6—i.e., up to as many as 200 miRNAs in total. Classifiers developed using the Set 6 markers are provided herein. Various types of mathematical models with appropriate parameters can be used in connection with this set of non-coding RNAs.

TABLE 6
hsa-miR-133a-5p
hsa-miR-3200-5p
hsa-miR-2277-3p
hsa-miR-6509-5p
hsa-miR-7109-3p
hsa-miR-372-3p
hsa-miR-365b-5p
hsa-miR-6839-5p
hsa-miR-6860
hsa-miR-193a-3p
hsa-miR-6838-3p
hsa-miR-6863
hsa-miR-541-5p
hsa-miR-517a-3p, or
hsa-miR-517b-3p
hsa-miR-6791-3p
hsa-miR-619-3p
hsa-miR-656-3p
hsa-miR-3591-3p
hsa-miR-6833-5p
hsa-miR-4710
hsa-miR-3913-5p
hsa-miR-6744-3p
hsa-miR-6737-3p
hsa-miR-4718
hsa-miR-4719
hsa-miR-3151-5p
hsa-miR-34c-3p
hsa-miR-1306-5p
hsa-miR-4652-3p
hsa-miR-4262
hsa-miR-6843-3p
hsa-miR-7151-5p
hsa-miR-1292-3p
hsa-miR-548ao-5p
hsa-miR-642b-3p
hsa-miR-6516-5p
hsa-miR-376c-3p
hsa-miR-7113-3p
hsa-miR-487a-3p
hsa-miR-3912-3p
hsa-miR-5008-5p
hsa-miR-130a-5p
hsa-miR-520c-3p
hsa-miR-7843-5p
hsa-miR-5003-5p
hsa-miR-6086
hsa-miR-6084
hsa-miR-6082
hsa-miR-6083
hsa-miR-6080

(7)

Classifiers Developed Using Set 7 Markers

Also provided herein is a set of non-coding RNAs (Set 7) that includes some or all non-coding RNAs of Set 6 and about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 7—i.e., up to as many as 250 miRNAs in total. Classifiers developed using the Set 7 markers are provided herein. Various types of mathematical models with appropriate parameters can be used in connection with this set of non-coding RNAs.

TABLE 7
hsa-miR-6081
hsa-miR-6847-3p
hsa-miR-5006-3p
hsa-miR-6743-3p
hsa-miR-607
hsa-miR-1296-3p
hsa-miR-4538
hsa-miR-4539
hsa-miR-603
hsa-miR-4535
hsa-miR-4537
hsa-miR-609
hsa-miR-4531
hsa-miR-4533
hsa-miR-5000-5p
hsa-miR-466
hsa-miR-3681-3p
hsa-miR-624-3p
hsa-miR-7153-5p
hsa-miR-561-5p
hsa-miR-6847-5p
hsa-miR-3153
hsa-miR-513c-3p
hsa-miR-6501-5p
hsa-miR-4646-5p
hsa-miR-3159
hsa-miR-6747-3p
hsa-miR-208a-3p
hsa-miR-548e-5p
hsa-miR-548d-3p
hsa-miR-381-3p
hsa-miR-892c-3p
hsa-miR-5189-3p
hsa-miR-942-3p
hsa-miR-6512-3p
hsa-miR-6813-3p
hsa-miR-6769b-3p
hsa-miR-3126-5p
hsa-miR-374c-5p
hsa-miR-4665-3p
hsa-miR-499a-3p
hsa-miR-4712-5p
hsa-miR-4697-5p
hsa-miR-4777-5p
hsa-miR-146a-3p
hsa-miR-3189-3p
hsa-miR-3939
hsa-miR-3938
hsa-miR-3935
hsa-miR-219a-2-3p

(8)

Classifiers Developed Using Set 8 Markers

Also provided herein is a set of non-coding RNAs (Set 8) that includes some or all non-coding RNAs of Set 7 and about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 8—i.e., up to as many as 300 miRNAs in total. Classifiers developed using the Set 8 markers are provided herein. Various types of mathematical models with appropriate parameters can be used in connection with this set of non-coding RNAs.

TABLE 8
hsa-miR-3936
hsa-miR-4684-5p
hsa-miR-4436b-3p
hsa-miR-4685-3p
hsa-miR-548aj-3p
hsa-miR-431-3p
hsa-miR-4661-3p
hsa-miR-1179
hsa-miR-4790-3p
hsa-miR-6893-3p
hsa-miR-200a-5p
hsa-miR-1273h-3p
hsa-miR-548av-3p
hsa-miR-766-3p
hsa-miR-6872-5p
hsa-miR-181d-5p
hsa-miR-888-5p
hsa-miR-4800-5p
hsa-miR-548f-5p
hsa-miR-124-3p
hsa-miR-6890-3p
hsa-miR-4694-5p
hsa-miR-181a-3p
hsa-miR-5701
hsa-miR-6830-5p
hsa-miR-550b-3p
hsa-miR-3171
hsa-miR-5571-3p
hsa-miR-3689f
hsa-miR-495-5p
hsa-miR-3689d
hsa-miR-545-3p
hsa-miR-4799-5p
hsa-miR-3065-5p
hsa-miR-105-5p
hsa-miR-4798-5p
hsa-miR-4778-5p
hsa-miR-4707-5p
hsa-miR-4433b-3p
hsa-miR-6845-5p
hsa-miR-548ap-3p
hsa-miR-3714
hsa-miR-302b-5p
hsa-miR-3713
hsa-miR-664b-3p
hsa-miR-549a
hsa-miR-5584-3p
hsa-miR-4704-5p
hsa-miR-188-3p
hsa-miR-4529-5p

(9)

Classifiers Developed Using Set 9 Markers

Table 9 includes 100 non-coding RNAs. Also provided herein is a set of non-coding RNAs (Set 9) that includes some or all non-coding RNAs of Set 8 and about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 non-coding RNAs selected from Table 9—i.e., up to as many as 400 miRNAs in total. Classifiers developed using the Set 9 markers are provided herein. Various types of mathematical models with appropriate parameters can be used in connection with this set of non-coding RNAs.

TABLE 9

| | | | |
|---|---|---|---|
| hsa-miR-6749-3p | hsa-miR-298 | hsa-miR-3675-5p | hsa-miR-6504-3p |
| hsa-miR-664a-3p | hsa-miR-297 | hsa-miR-216a-5p | hsa-miR-1271-5p |
| hsa-miR-6865-5p | hsa-miR-4640-5p | hsa-miR-31-3p | hsa-miR-7157-3p |
| hsa-miR-4705 | hsa-miR-1468-3p | hsa-miR-6828-3p | hsa-miR-2467-5p |
| hsa-miR-3120-3p | hsa-miR-3152-5p | hsa-miR-4722-5p | hsa-miR-215-3p |
| hsa-miR-518a-3p | hsa-miR-488-3p | hsa-miR-3168 | hsa-miR-6785-3p |
| hsa-miR-5587-3p | hsa-miR-744-3p | hsa-miR-3169 | hsa-miR-4802-5p |
| hsa-miR-6811-3p | hsa-miR-612 | hsa-miR-3166 | hsa-miR-25-5p |
| hsa-miR-138-1-3p | hsa-miR-613 | hsa-miR-3167 | hsa-miR-218-2-3p |
| hsa-miR-2392 | hsa-miR-610 | hsa-miR-3164 | hsa-miR-1288-3p |
| hsa-miR-371b-3p | hsa-miR-611 | hsa-miR-3165 | hsa-miR-6727-3p |
| hsa-miR-5089-3p | hsa-miR-450b-5p | hsa-miR-3163 | hsa-miR-7855-5p |
| hsa-miR-519b-3p | hsa-miR-617 | hsa-miR-3161 | hsa-miR-7159-3p |
| hsa-miR-302a-5p | hsa-miR-6508-5p | hsa-miR-3136-5p | hsa-miR-4330 |
| hsa-miR-651-5p | hsa-miR-5580-3p | hsa-miR-6799-3p | hsa-miR-4768-5p |
| hsa-miR-6502-5p | hsa-miR-100-5p | hsa-miR-507 | hsa-miR-548au-5p |
| hsa-miR-514b-3p | hsa-miR-4528 | hsa-miR-1225-5p | hsa-miR-1228-3p |
| hsa-miR-654-3p | hsa-miR-510-3p | hsa-miR-605-3p | hsa-miR-3941 |
| hsa-miR-4637 | hsa-miR-4526 | hsa-miR-636 | hsa-miR-3943 |
| hsa-miR-4636 | hsa-miR-4523 | hsa-miR-1224-3p | hsa-miR-3945 |
| hsa-miR-4635 | hsa-miR-4522 | hsa-miR-6716-3p | hsa-miR-8053 |
| hsa-miR-367-3p | hsa-miR-4521 | hsa-miR-1277-3p | hsa-miR-8052 |
| hsa-miR-518e-3p | hsa-miR-520g-3p | hsa-miR-4711-5p | hsa-miR-6761-5p |
| hsa-miR-652-5p | hsa-miR-4709-3p | hsa-miR-3064-5p | hsa-miR-8057 |
| hsa-miR-6815-3p | hsa-miR-373-5p | hsa-miR-6739-3p | hsa-miR-8056 |

(10)

Classifiers Developed Using the Set 10 Markers

Table 10 includes 100 non-coding RNAs. Also provided herein is a set of non-coding RNAs (Set 10) that includes some or all non-coding RNAs of Set 9 and about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 non-coding RNAs selected from Table 10—i.e., up to as many as 500 miRNAs in total. Classifiers developed using the Set 10 markers are provided herein. Various types of mathematical models with appropriate parameters can be used in connection with this set of non-coding RNAs. In some embodiments, Set 10 comprises from 401 to 500 miRNAs.

TABLE 10

| | | | |
|---|---|---|---|
| hsa-miR-8055 | hsa-miR-597-5p | hsa-miR-2115-3p | hsa-miR-4511 |
| hsa-miR-6781-3p | hsa-miR-133a-3p | hsa-miR-548n | hsa-miR-4516 |
| hsa-miR-8058 | hsa-miR-138-5p | hsa-miR-548m | hsa-miR-3150a-3p |
| hsa-miR-7161-3p | hsa-miR-6778-3p | hsa-miR-548l | hsa-miR-4514 |
| hsa-miR-3677-5p | hsa-miR-1236-3p | hsa-miR-548k | hsa-miR-4515 |
| hsa-miR-4666a-5p | hsa-miR-1266-5p | hsa-miR-548i | hsa-miR-550a-5p |
| hsa-miR-6841-3p | hsa-miR-323a-3p | hsa-miR-642b-5p | hsa-miR-409-5p |
| hsa-miR-6856-3p | hsa-miR-4536-5p | hsa-miR-376c-5p | hsa-miR-5100 |
| hsa-miR-6796-5p | hsa-miR-6838-5p | hsa-miR-548y | hsa-miR-4677-5p |
| hsa-miR-3144-3p | hsa-miR-6844 | hsa-miR-548w | hsa-miR-6503-5p |
| hsa-miR-513b-5p | hsa-miR-132-3p | hsa-miR-548v | hsa-miR-6755-3p |
| hsa-miR-4756-3p | hsa-miR-670-3p | hsa-miR-548u | hsa-miR-7153-3p |
| hsa-miR-6510-3p | hsa-miR-4436a | hsa-miR-5739 | hsa-miR-7160-5p |
| hsa-miR-6730-5p | hsa-miR-1304-5p | hsa-miR-548q | hsa-miR-6793-3p |
| hsa-miR-1260a | hsa-miR-4762-3p | hsa-miR-548p | hsa-miR-5002-5p |
| hsa-miR-6867-5p | hsa-miR-6827-5p | hsa-miR-9-5p | hsa-miR-3179 |
| hsa-miR-574-5p | hsa-miR-4782-3p | hsa-miR-598-5p | hsa-miR-128-3p |
| hsa-miR-181b-3p | hsa-miR-4737 | hsa-miR-4793-5p | hsa-miR-95-3p |
| hsa-miR-4755-3p | hsa-miR-2116-3p | hsa-miR-6068 | hsa-miR-451b |
| hsa-miR-4655-5p | hsa-miR-1301-3p | hsa-miR-6069 | hsa-miR-3170 |
| hsa-miR-4786-3p | hsa-miR-5195-5p | hsa-miR-662 | hsa-miR-642a-5p |
| hsa-miR-3140-3p | hsa-miR-1307-5p | hsa-miR-4518 | hsa-miR-451a |
| hsa-miR-4753-5p | hsa-miR-3146 | hsa-miR-4519 | hsa-miR-3175 |
| hsa-miR-146a-5p | hsa-miR-3689b-3p, or | hsa-miR-4512 | hsa-miR-3174 |
| | hsa-miR-3689c | | |
| hsa-miR-6869-3p | hsa-miR-4667-5p | hsa-miR-4510 | hsa-miR-3176 |

(11)

Classifiers Developed Using the Set 11 Markers

Table 11 includes 100 non-coding RNAs. Also provided herein is a set of non-coding RNAs (Set 11) that includes some or all non-coding RNAs of Set 10 and about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 non-coding RNAs selected from Table 11—i.e., as many as 600 miRNAs in total. Classifiers developed using the Set 11 markers are provided herein. Various types of mathematical models with appropriate parameters can be used in connection with this set of non-coding RNAs. In some embodiments, Set 11 comprises from 501 to 600 miRNAs.

TABLE 11

| | | | |
|---|---|---|---|
| hsa-miR-4720-5p | hsa-miR-331-3p | hsa-miR-4285 | hsa-miR-4472 |
| hsa-miR-4646-3p | hsa-miR-6852-3p | hsa-miR-4288 | hsa-miR-6804-5p |
| hsa-miR-514a-5p | hsa-miR-219a-5p | hsa-miR-4289 | hsa-miR-4798-3p |
| hsa-miR-6753-5p | hsa-miR-452-3p | hsa-miR-7852-3p | hsa-miR-615-3p |
| hsa-miR-6500-5p | hsa-miR-6133 | hsa-miR-127-5p | hsa-miR-3605-5p |
| hsa-miR-4781-3p | hsa-miR-3130-3p | hsa-miR-6718-5p | hsa-miR-1911-3p |
| hsa-miR-541-3p | hsa-miR-23b-5p | hsa-miR-4479 | hsa-miR-664b-5p |
| hsa-miR-6735-3p | hsa-miR-4777-3p | hsa-miR-4478 | hsa-miR-6733-3p |
| hsa-miR-106a-5p | hsa-miR-4766-3p | hsa-miR-766-5p | hsa-miR-493-3p |
| hsa-miR-7155-3p | hsa-miR-4713-5p | hsa-miR-3922-3p | hsa-miR-548b-3p |
| hsa-miR-548d-5p | hsa-miR-524-3p | hsa-miR-6758-5p | hsa-miR-7706 |
| hsa-miR-381-5p | hsa-miR-3691-5p | hsa-miR-888-3p | hsa-miR-7705 |
| hsa-miR-3117-5p | hsa-miR-4632-3p | hsa-miR-6759-3p | hsa-miR-7702 |
| hsa-miR-5189-5p | hsa-miR-8067 | hsa-miR-3664-5p | hsa-miR-7703 |
| hsa-miR-942-5p | hsa-miR-548x-3p | hsa-miR-548f-3p | hsa-miR-6865-3p |
| hsa-miR-6848-3p | hsa-miR-8065 | hsa-miR-6733-5p | hsa-miR-5579-3p |
| hsa-miR-30c-1-3p | hsa-miR-520f-5p | hsa-miR-5589-5p | hsa-miR-4729 |
| hsa-miR-548j-3p | hsa-miR-8060 | hsa-miR-5591-5p | hsa-miR-1245a |
| hsa-miR-3126-3p | hsa-miR-4716-3p | hsa-miR-4695-3p | hsa-miR-20a-3p |
| hsa-miR-4691-3p | hsa-miR-383-5p | hsa-miR-4743-5p | hsa-miR-4721 |
| hsa-miR-103b | hsa-miR-4283 | hsa-miR-6748-5p | hsa-miR-380-5p |
| hsa-miR-342-3p | hsa-miR-4280 | hsa-miR-5571-5p | hsa-miR-6814-5p |
| hsa-miR-5187-3p | hsa-miR-4281 | hsa-miR-648 | hsa-miR-3649 |
| hsa-miR-4774-3p | hsa-miR-4287 | hsa-miR-6846-5p | hsa-miR-5010-3p |
| hsa-miR-1238-3p | hsa-miR-4284 | hsa-miR-6742-5p | hsa-miR-3648 |

(12)

Classifiers Developed Using the Set 12 Markers

Table 12 includes 200 non-coding RNAs. Also provided herein is a set of non-coding RNAs (Set 12) that includes some or all non-coding RNAs of Set 11 and about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, or 200 non-coding RNAs selected from Table 12—i.e., as many as 800 miRNAs in total. Classifiers developed using the Set 12 markers are provided herein. Various types of mathematical models with appropriate parameters can be used in connection with this set of non-coding RNAs. In some embodiments, Set 12 comprises from 601 to 800 miRNAs.

TABLE 12-1
Table 12

| | | | |
|---|---|---|---|
| hsa-miR-660-3p | hsa-miR-2681-5p | hsa-miR-19b-2-5p | hsa-miR-645 |
| hsa-miR-6767-3p | hsa-miR-4763-5p | hsa-miR-5196-3p | hsa-miR-647 |
| hsa-miR-497-3p | hsa-miR-4639-3p | hsa-miR-376b-3p | hsa-miR-646 |
| hsa-miR-1207-3p | hsa-miR-5583-5p | hsa-miR-2909 | hsa-miR-649 |
| hsa-miR-6832-5p | hsa-miR-1271-3p | hsa-miR-4645-5p | hsa-miR-2114-5p |
| hsa-miR-6884-5p | hsa-miR-5582-5p | hsa-miR-6797-5p | hsa-miR-29a-5p |
| hsa-miR-6502-3p | hsa-miR-503-5p | hsa-miR-6763-3p | hsa-miR-5001-3p |
| hsa-miR-6885-3p | hsa-miR-512-5p | hsa-miR-6750-5p | hsa-miR-6855-5p |
| hsa-miR-3151-3p | hsa-miR-6862-3p | hsa-miR-597-3p | hsa-miR-19b-1-5p |
| hsa-miR-3607-5p | hsa-miR-345-3p | hsa-miR-4315 | hsa-miR-4436b-5p |
| hsa-miR-6861-3p | hsa-miR-10b-3p | hsa-miR-6715b-3p | hsa-miR-3127-5p |
| hsa-miR-6774-5p | hsa-miR-890 | hsa-miR-668-3p | hsa-miR-4423-5p |
| hsa-miR-519d-3p | hsa-miR-4650-3p | hsa-miR-3944-5p | hsa-miR-2682-3p |
| hsa-miR-367-5p | hsa-miR-7159-5p | hsa-miR-6731-5p | hsa-miR-5002-3p |
| hsa-miR-222-5p | hsa-miR-4768-3p | hsa-miR-323a-5p | hsa-miR-519c-3p |
| hsa-miR-6801-3p | hsa-miR-105-3p | hsa-miR-6798-3p | hsa-miR-98-5p |
| hsa-miR-3152-3p | hsa-miR-6505-3p | hsa-miR-520d-5p | hsa-miR-3193 |
| hsa-miR-6077 | hsa-miR-8079 | hsa-miR-670-5p | hsa-miR-4761-5p |
| hsa-miR-6076 | hsa-miR-8078 | hsa-miR-4762-5p | hsa-miR-644a |
| hsa-miR-6074 | hsa-miR-4299 | hsa-miR-4759 | hsa-miR-548az-3p |
| hsa-miR-6073 | hsa-miR-3194-3p | hsa-miR-4676-5p | hsa-miR-187-3p |
| hsa-miR-6072 | hsa-miR-4295 | hsa-miR-22-5p | hsa-miR-7114-3p |
| hsa-miR-6071 | hsa-miR-4294 | hsa-miR-4751 | hsa-miR-3199 |
| hsa-miR-6070 | hsa-miR-4297 | hsa-miR-4752 | hsa-miR-3198 |
| hsa-miR-6751-3p | hsa-miR-4296 | hsa-miR-4754 | hsa-miR-6753-3p |
| hsa-miR-6078 | hsa-miR-4291 | hsa-miR-3190-3p | hsa-miR-4670-3p |
| hsa-miR-6745 | hsa-miR-8074 | hsa-miR-4701-3p | hsa-miR-4781-5p |

TABLE 12-2

| | | | |
|---|---|---|---|
| hsa-miR-6807-5p | hsa-miR-4293 | hsa-miR-2116-5p | hsa-miR-4667-3p |
| hsa-miR-6894-5p | hsa-miR-4292 | hsa-miR-219a-1-3p | hsa-miR-132-5p |
| hsa-miR-5580-5p | hsa-miR-7161-5p | hsa-miR-4699-5p | hsa-miR-4731-3p |
| hsa-miR-516b-5p | hsa-miR-4736 | hsa-miR-1295a | hsa-miR-520a-3p |
| hsa-miR-4504 | hsa-miR-6780a-3p | hsa-miR-7108-3p | hsa-miR-675-3p |
| hsa-miR-510-5p | hsa-miR-6506-3p | hsa-miR-449b-5p | hsa-miR-526a, |
| | | | hsa-miR-520c-5p, or |
| | | | hsa-miR-518d-5p |
| hsa-miR-4506 | hsa-miR-4714-3p | hsa-miR-6824-3p | hsa-miR-196b-3p |
| hsa-miR-4474-3p | hsa-miR-6881-3p | hsa-miR-122-3p | hsa-miR-6130 |
| hsa-miR-4500 | hsa-miR-4761-3p | hsa-miR-449c-5p | hsa-miR-6850-3p |
| hsa-miR-4503 | hsa-miR-6856-5p | hsa-miR-876-3p | hsa-miR-219b-3p |
| hsa-miR-4502 | hsa-miR-155-3p | hsa-miR-1295b-3p | hsa-miR-6134 |
| hsa-miR-487b-3p | hsa-miR-520g-5p | hsa-miR-3975 | hsa-miR-3619-5p |
| hsa-miR-4509 | hsa-miR-1229-3p | hsa-miR-1227-3p | hsa-miR-6741-3p |
| hsa-miR-4709-5p | hsa-miR-6515-5p | hsa-miR-3186-3p | hsa-miR-1587 |
| hsa-miR-582-3p | hsa-miR-4756-5p | hsa-miR-7152-5p | hsa-miR-5590-5p |
| hsa-miR-3150b-3p | hsa-miR-328-3p | hsa-miR-7158-5p | hsa-miR-676-3p |
| hsa-miR-3181 | hsa-miR-7110-3p | hsa-miR-6883-5p | hsa-miR-4742-5p |
| hsa-miR-3182 | hsa-miR-6820-3p | hsa-miR-3613-3p | hsa-miR-421 |
| hsa-miR-3183 | hsa-miR-6809-3p | hsa-miR-3928-5p | hsa-miR-4764-3p |
| hsa-miR-5581-5p | hsa-miR-1287-3p | hsa-miR-6842-3p | hsa-miR-196a-3p |
| hsa-miR-1224-5p | hsa-miR-3614-3p | hsa-miR-641 | hsa-miR-323b-5p |
| hsa-miR-300 | hsa-miR-5197-5p | hsa-miR-640 | hsa-miR-4789-3p |
| hsa-miR-3160-3p | hsa-miR-4255 | hsa-miR-643 | hsa-miR-3942-3p |

(13)

Classifiers Developed Using Set 13 Markers

Table 13 includes 200 non-coding RNAs. Also provided herein is a set of non-coding RNAs (Set 13) that includes some or all non-coding RNAs of Set 12 and about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, or 200 non-coding RNAs selected from Table 13—i.e., as many as 1000 miRNAs in total. Classifiers developed using the Set 13 markers are provided herein. Various types of mathematical models with appropriate parameters can be used in connection with this set of non-coding RNAs. In some embodiments, Set 13 comprises from 801 to 1000 miRNAs.

TABLE 13-1
Table 13

| | | | |
|---|---|---|---|
| hsa-miR-6769a-3p | hsa-miR-4659a-3p | hsa-miR-6807-3p | hsa-miR-3194-5p |
| hsa-miR-548s | hsa-miR-526b-5p | hsa-miR-657 | hsa-miR-3162-5p |
| hsa-miR-7106-3p | hsa-miR-5579-5p | hsa-miR-548am-3p | hsa-miR-3156-5p |
| hsa-miR-506-3p | hsa-miR-32-3p | hsa-miR-4659b-3p | hsa-miR-548ai, |
| | | | hsa-miR-570-5p |
| hsa-miR-5009-3p | hsa-miR-3622b-3p | hsa-miR-6895-3p | hsa-miR-579-3p |
| hsa-miR-1537-3p | hsa-miR-4748 | hsa-miR-129-1-3p | hsa-miR-937-3p |
| hsa-miR-4783-5p | hsa-miR-494-5p | hsa-miR-4517 | hsa-miR-3157-5p |
| hsa-miR-203b-5p | hsa-miR-4744 | hsa-miR-1250-3p | hsa-miR-450a-1-3p |
| hsa-miR-548ah-5p | hsa-miR-5693 | hsa-miR-92a-1-5p | hsa-miR-873-5p |
| hsa-miR-8085 | hsa-miR-5690 | hsa-miR-582-5p | hsa-miR-4735-3p |
| hsa-miR-3973 | hsa-miR-5691 | hsa-miR-125b-2-3p | hsa-miR-450a-2-3p |
| hsa-miR-3972 | hsa-miR-5696 | hsa-miR-6800-3p | hsa-miR-921 |
| hsa-miR-8080 | hsa-miR-5697 | hsa-miR-4769-5p | hsa-miR-922 |
| hsa-miR-3974 | hsa-miR-5694 | hsa-miR-6835-3p | hsa-miR-924 |
| hsa-miR-3977 | hsa-miR-5695 | hsa-miR-1178-5p | hsa-miR-4725-5p |
| hsa-miR-8083 | hsa-miR-5698 | hsa-miR-6720-3p | hsa-miR-6783-5p |
| hsa-miR-3978 | hsa-miR-33b-5p | hsa-miR-4740-3p | hsa-miR-155-5p |
| hsa-miR-383-3p | hsa-miR-4501 | hsa-miR-6803-3p | hsa-miR-125b-1-3p |
| hsa-miR-8088 | hsa-miR-6734-5p | hsa-miR-4418 | hsa-miR-18b-3p |
| hsa-miR-8089 | hsa-miR-4772-5p | hsa-miR-4417 | hsa-miR-6768-5p |
| hsa-miR-3940-3p | hsa-miR-302c-5p | hsa-miR-4419a | hsa-miR-6874-3p |
| hsa-miR-218-1-3p | hsa-miR-6782-3p | hsa-miR-3160-5p | hsa-miR-1229-5p |
| hsa-miR-200b-5p | hsa-miR-212-3p | hsa-miR-325 | hsa-miR-3690 |
| hsa-miR-519d-5p | hsa-miR-1913 | hsa-miR-6878-3p | hsa-miR-4653-3p |
| hsa-miR-7977 | hsa-miR-1912 | hsa-miR-4668-5p | hsa-miR-3663-5p |
| hsa-miR-4664-3p | hsa-miR-26a-1-3p | hsa-miR-6127 | hsa-miR-6794-3p |
| hsa-miR-376a-2-5p | hsa-miR-518c-5p | hsa-miR-938 | hsa-miR-6728-3p |
| hsa-miR-6760-5p | hsa-miR-3691-3p | hsa-miR-3617-3p | hsa-miR-6866-3p |

TABLE 13-2

| | | | |
|---|---|---|---|
| hsa-miR-4680-3p | hsa-miR-1185-1-3p | hsa-miR-6129 | hsa-miR-5196-5p |
| hsa-miR-936 | hsa-miR-103a-2-5p | hsa-miR-6831-3p | hsa-miR-1256 |
| hsa-miR-4687-5p | hsa-miR-134-3p | hsa-miR-1226-5p | hsa-miR-379-5p |
| hsa-miR-6783-3p | hsa-miR-6832-3p | hsa-miR-3180-5p | hsa-miR-6514-3p |
| hsa-miR-4738-5p | hsa-miR-2113 | hsa-miR-6886-5p | hsa-miR-6507-5p |
| hsa-miR-6759-5p | hsa-miR-8086 | hsa-miR-5582-3p | hsa-miR-6732-3p |
| hsa-miR-3664-3p | hsa-miR-8087 | hsa-miR-4684-3p | hsa-miR-135b-5p |
| hsa-miR-4524a-5p | hsa-miR-588 | hsa-miR-512-3p | hsa-miR-6797-3p |
| hsa-miR-6819-3p | hsa-miR-6754-3p | hsa-miR-6862-5p | hsa-miR-6786-3p |
| hsa-miR-3606-5p | hsa-miR-3607-3p | hsa-miR-502-5p | hsa-miR-208a-5p |
| hsa-miR-182-5p | hsa-miR-8081 | hsa-miR-542-3p | hsa-miR-6888-3p |
| hsa-miR-183-3p | hsa-miR-4724-3p | hsa-miR-423-3p | hsa-miR-3200-3p |
| hsa-miR-6846-3p | hsa-miR-100-3p | hsa-miR-3667-3p | hsa-miR-6509-3p |
| hsa-miR-548a-5p | hsa-miR-6880-3p | hsa-miR-935 | hsa-miR-4662a-3p |
| hsa-miR-891a-3p | hsa-miR-3976 | hsa-miR-5007-5p | hsa-miR-3944-3p |
| hsa-miR-1914-5p | hsa-miR-653-3p | hsa-miR-4445-3p | hsa-miR-3529-5p |
| hsa-miR-590-5p | hsa-miR-5586-3p | hsa-miR-4685-5p | hsa-miR-6839-3p |
| hsa-miR-511-5p | hsa-miR-223-5p | hsa-miR-3192-3p | hsa-miR-6715b-5p |
| hsa-miR-1827 | hsa-miR-4750-3p | hsa-miR-659-3p | hsa-miR-6731-3p |
| hsa-miR-889-5p | hsa-miR-6757-3p | hsa-miR-6505-5p | hsa-miR-199a-3p_{,} or |
| | | | hsa-miR-199b-3p |
| hsa-miR-6891-3p | hsa-miR-6773-3p | hsa-miR-3927-3p | hsa-miR-1255a |
| hsa-miR-548t-5p | hsa-miR-6751-5p | hsa-miR-6738-3p | hsa-miR-548ar-5p |

(14)

Classifiers Developed Using the Set 14 Markers

Table 14 includes 1004 non-coding RNAs. Also provided herein is a set of non-coding RNAs (Set 14) that includes some or all non-coding RNAs of Set 13 and about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, or 1004 non-coding RNAs selected from Table 14—i.e., as many as 2004 miRNAs in total. Classifiers developed using the Set 13 markers are provided herein. Various types of mathematical models with appropriate parameters can be used in connection with this set of non-coding RNAs. In some embodiments, Set 14 comprises from 1001 to 2004 miRNAs.

TABLE 14-1
Table 14

| | | | |
|---|---|---|---|
| hsa-miR-4747-3p | hsa-miR-378f | hsa-miR-4797-5p | hsa-miR-4470 |
| hsa-miR-345-5p | hsa-miR-378g | hsa-miR-4716-5p | hsa-miR-4473 |
| hsa-miR-379-3p | hsa-miR-2276-3p | hsa-miR-6884-3p | hsa-miR-761 |
| hsa-miR-6854-5p | hsa-miR-378e | hsa-miR-6802-3p | hsa-miR-4475 |
| hsa-miR-548ba | hsa-miR-378j | hsa-miR-548g-5p, | hsa-miR-764 |
| | | hsa-miR-548x-5p, or | |
| | | hsa-miR-548aj-5p | |
| hsa-miR-4676-3p | hsa-miR-378h | hsa-miR-5091 | hsa-miR-765 |
| hsa-miR-4775 | hsa-miR-4653-5p | hsa-miR-5092 | hsa-miR-555 |
| hsa-miR-5689 | hsa-miR-24-2-5p | hsa-miR-6837-3p | hsa-miR-4679 |
| hsa-miR-5688 | hsa-miR-1278 | hsa-miR-5094 | hsa-miR-4678 |
| hsa-miR-4770 | hsa-miR-1279 | hsa-miR-5095 | hsa-miR-4673 |
| hsa-miR-4771 | hsa-miR-1270 | hsa-miR-5096 | hsa-miR-4672 |
| hsa-miR-5685 | hsa-miR-1272 | hsa-miR-576-3p | hsa-miR-6774-3p |
| hsa-miR-5684 | hsa-miR-6784-3p | hsa-miR-33a-5p | hsa-miR-543 |
| hsa-miR-5687 | hsa-miR-1276 | hsa-miR-4727-3p | hsa-miR-371a-3p |
| hsa-miR-4701-5p | hsa-miR-4303 | hsa-miR-378d | hsa-miR-6829-3p |
| hsa-miR-5680 | hsa-miR-4302 | hsa-miR-99a-3p | hsa-miR-6801-5p |
| hsa-miR-5683 | hsa-miR-4301 | hsa-miR-548h-5p | hsa-miR-6511b-3p |
| hsa-miR-4787-3p | hsa-miR-4300 | hsa-miR-548as-3p | hsa-miR-20b-3p |
| hsa-miR-34c-5p | hsa-miR-4307 | hsa-miR-4649-5p | hsa-miR-1539 |
| hsa-miR-556-5p | hsa-miR-4305 | hsa-miR-4758-3p | hsa-miR-1538 |
| hsa-miR-1306-3p | hsa-miR-4304 | hsa-miR-378i | hsa-miR-498 |
| hsa-miR-4760-5p | hsa-miR-4309 | hsa-miR-6857-5p | hsa-miR-496 |
| hsa-miR-1253 | hsa-miR-4308 | hsa-miR-128-2-5p | hsa-miR-492 |
| hsa-miR-5093 | hsa-miR-7112-5p | hsa-miR-6506-5p | hsa-miR-3622a-3p |
| hsa-miR-4272 | hsa-miR-6822-5p | hsa-miR-4714-5p | hsa-miR-134-5p |
| hsa-miR-516b-3p, or | hsa-miR-2110 | hsa-miR-6833-3p | hsa-miR-525-5p |
| hsa-miR-516a-3p | | | |
| hsa-miR-506-5p | hsa-miR-4755-5p | hsa-miR-194-3p | hsa-miR-6894-3p |
| hsa-miR-145-3p | hsa-miR-6507-3p | hsa-miR-3616-3p | hsa-miR-518b |
| hsa-miR-449c-3p | hsa-miR-6726-3p | hsa-miR-1277-5p | hsa-miR-3155b |
| hsa-miR-1292-5p | hsa-miR-135b-3p | hsa-miR-892b | hsa-miR-3155a |

TABLE 14-2

| | | | |
|---|---|---|---|
| hsa-miR-6790-3p | hsa-miR-520b | hsa-miR-892a | hsa-miR-584-3p |
| hsa-miR-190a-5p | hsa-miR-520e | hsa-miR-4711-3p | hsa-miR-6787-3p |
| hsa-miR-6777-3p | hsa-miR-3140-5p | hsa-miR-7844-5p | hsa-miR-5588-3p |
| hsa-miR-6858-3p | hsa-miR-520h | hsa-miR-2276-5p | hsa-miR-3192-5p |
| hsa-miR-7113-5p | hsa-miR-6868-5p | hsa-miR-1266-3p | hsa-miR-659-5p |
| hsa-miR-1234-3p | hsa-miR-6888-5p | hsa-miR-1237-3p | hsa-miR-6764-3p |
| hsa-miR-1273a | hsa-miR-5681a | hsa-miR-4329 | hsa-miR-627-5p |
| hsa-miR-3912-5p | hsa-miR-5681b | hsa-miR-1257 | hsa-miR-5692a |
| hsa-miR-1273c | hsa-miR-1305 | hsa-miR-1254 | hsa-miR-5692c |
| hsa-miR-1273d | hsa-miR-4536-3p | hsa-miR-5197-3p | hsa-miR-5692b |
| hsa-miR-1273e | hsa-miR-1302 | hsa-miR-4321 | hsa-miR-6738-5p |
| hsa-miR-1273f | hsa-miR-4662a-5p | hsa-miR-4320 | hsa-miR-6882-5p |
| hsa-miR-6736-3p | hsa-miR-181c-5p | hsa-miR-4323 | hsa-miR-3128 |
| hsa-miR-758-5p | hsa-miR-4802-3p | hsa-miR-4322 | hsa-miR-384 |
| hsa-miR-4279 | hsa-miR-628-3p | hsa-miR-4325 | hsa-miR-3122 |
| hsa-miR-3613-5p | hsa-miR-365a-3p, or | hsa-miR-4327 | hsa-miR-3123 |
| | hsa-miR-365b-3p | | |
| hsa-miR-7515 | hsa-miR-6854-3p | hsa-miR-4326 | hsa-miR-579-5p |
| hsa-miR-2355-5p | hsa-miR-548ay-3p | hsa-miR-7112-3p | hsa-miR-4252 |
| hsa-miR-3616-5p | hsa-miR-4650-5p | hsa-miR-143-5p | hsa-miR-3125 |
| hsa-miR-5006-5p | hsa-miR-509-3-5p | hsa-miR-4645-3p | hsa-miR-6790-5p |
| hsa-miR-4703-5p | hsa-miR-625-5p | hsa-miR-6750-3p | hsa-miR-873-3p |
| hsa-miR-2114-3p | hsa-miR-4782-5p | hsa-miR-16-2-3p | hsa-miR-5186 |
| hsa-miR-3202 | hsa-miR-548ao-3p | hsa-miR-548c-5p, | hsa-miR-7978 |
| | | hsa-miR-548o-5p, or | |
| | | hsa-miR-548am-5p | |
| hsa-miR-3201 | hsa-miR-6772-3p | hsa-miR-6868-3p | hsa-miR-7975 |
| hsa-miR-1255b-5p | hsa-miR-1301-5p | hsa-miR-3074-5p | hsa-miR-7974 |
| hsa-miR-3620-3p | hsa-miR-3145-3p | hsa-miR-4747-5p | hsa-miR-944 |
| hsa-miR-4794 | hsa-miR-6808-3p | hsa-miR-605-5p | hsa-miR-7976 |
| hsa-miR-4791 | hsa-miR-7-5p | hsa-miR-1322 | hsa-miR-943 |
| hsa-miR-624-5p | hsa-miR-3658 | hsa-miR-1323 | hsa-miR-7973 |
| hsa-miR-4423-3p | hsa-miR-3659 | hsa-miR-1321 | hsa-miR-2681-3p |

TABLE 14-3

| | | | |
|---|---|---|---|
| hsa-miR-373-3p | hsa-miR-4760-3p | hsa-miR-1324 | hsa-miR-6875-3p |
| hsa-miR-6501-3p | hsa-miR-3655 | hsa-miR-3677-3p | hsa-miR-6768-3p |
| hsa-miR-329-5p | hsa-miR-3657 | hsa-miR-370-3p | hsa-miR-4639-5p |
| hsa-miR-3925-5p | hsa-miR-4700-5p | hsa-miR-625-3p | hsa-miR-5583-3p |
| hsa-miR-7849-3p | hsa-miR-3651 | hsa-miR-8054 | hsa-miR-4638-3p |
| hsa-miR-4428 | hsa-miR-3652 | hsa-miR-548ay-5p | hsa-miR-141-5p |
| hsa-miR-4422 | hsa-miR-522-3p | hsa-miR-6772-5p | hsa-miR-708-3p |
| hsa-miR-4420 | hsa-miR-6719-3p | hsa-miR-6796-3p | hsa-miR-520d-3p |
| hsa-miR-4421 | hsa-miR-518d-3p | hsa-miR-550b-2-5p | hsa-miR-4269 |
| hsa-miR-4426 | hsa-miR-2115-5p | hsa-miR-606 | hsa-miR-7854-3p |
| hsa-miR-4427 | hsa-miR-6771-3p | hsa-miR-31-5p | hsa-miR-3689a-3p |
| hsa-miR-4424 | hsa-miR-4446-5p | hsa-miR-7154-3p | hsa-miR-193b-5p |
| hsa-miR-4425 | hsa-miR-6514-5p | hsa-miR-513b-3p | hsa-miR-3158-3p |
| hsa-miR-4731-5p | hsa-miR-769-5p | hsa-miR-3670 | hsa-miR-515-3p |
| hsa-miR-675-5p | hsa-miR-424-3p | hsa-miR-519c-5p, | hsa-miR-6780a-5p |
| | | hsa-miR-523-5p, | |
| | | hsa-miR-518e-5p, | |
| | | hsa-miR-522-5p, | |
| | | hsa-miR-519a-5p, or | |
| | | hsa-miR-519b-5p | |
| hsa-miR-3173-5p | hsa-miR-3913-3p | hsa-miR-3674 | hsa-miR-3074-3p |
| hsa-miR-376a-3p | hsa-miR-758-3p | hsa-miR-6128 | hsa-miR-3529-3p |
| hsa-miR-195-3p | hsa-miR-6500-3p | hsa-miR-4804-5p | hsa-miR-3907 |
| hsa-miR-6876-5p | hsa-miR-3129-3p | hsa-miR-6849-3p | hsa-miR-3908 |
| hsa-miR-153-3p | hsa-miR-6779-3p | hsa-miR-330-5p | hsa-miR-3909 |
| hsa-miR-655-5p | hsa-miR-2277-5p | hsa-miR-8066 | hsa-miR-4273 |
| hsa-miR-5590-3p | hsa-miR-147a | hsa-miR-34a-3p | hsa-miR-3614-5p |
| hsa-miR-4742-3p | hsa-miR-500b-5p | hsa-miR-574-3p | hsa-miR-4277 |
| hsa-miR-4671-3p | hsa-miR-6747-5p | hsa-miR-4686 | hsa-miR-4276 |
| hsa-miR-4789-5p | hsa-miR-6853-3p | hsa-miR-4682 | hsa-miR-4275 |
| hsa-miR-4693-5p | hsa-miR-4795-5p | hsa-miR-4683 | hsa-miR-4274 |
| hsa-miR-4757-5p | hsa-miR-4703-3p | hsa-miR-4681 | hsa-miR-1184 |
| hsa-miR-3150b-5p | hsa-miR-4726-3p | hsa-miR-769-3p | hsa-miR-4278 |

TABLE 14-4

| | | | |
|---|---|---|---|
| hsa-miR-3189-5p | hsa-miR-5682 | hsa-miR-3922-5p | hsa-miR-1182 |
| hsa-miR-410-3p | hsa-miR-656-5p | hsa-miR-34b-3p | hsa-miR-1183 |
| hsa-miR-302f | hsa-miR-3591-5p | hsa-miR-7158-3p | hsa-miR-1181 |
| hsa-miR-302e | hsa-miR-550a-3p | hsa-miR-589-3p | hsa-miR-370-5p |
| hsa-miR-1298-5p | hsa-miR-6859-5p | hsa-miR-3129-5p | hsa-miR-6881-5p |
| hsa-miR-4524b-3p | hsa-miR-6737-5p | hsa-miR-4690-3p | hsa-miR-548z, or |
| | | | hsa-miR-548h-3p |
| hsa-miR-302d-3p | hsa-miR-7160-3p | hsa-miR-8064 | hsa-miR-487a-5p |
| hsa-miR-508-5p | hsa-miR-4445-5p | hsa-miR-7111-3p | hsa-miR-6736-5p |
| hsa-miR-4661-5p | hsa-miR-202-5p | hsa-miR-1236-5p | hsa-miR-7152-3p |
| hsa-miR-527, or | hsa-miR-6792-5p | hsa-miR-4720-3p | hsa-miR-3187-3p |
| hsa-miR-518a-5p | | | |
| hsa-miR-767-3p | hsa-miR-1470 | hsa-miR-3928-3p | hsa-miR-539-5p |
| hsa-miR-4776-3p | hsa-miR-1471 | hsa-miR-500b-3p | hsa-miR-138-2-3p |
| hsa-miR-1245b-3p | hsa-miR-4662b | hsa-miR-6853-5p | hsa-miR-887-5p |
| hsa-miR-7850-5p | hsa-miR-4652-5p | hsa-miR-106a-3p | hsa-miR-7154-5p |
| hsa-miR-7851-3p | hsa-miR-6857-3p | hsa-miR-4795-3p | hsa-miR-210-3p |
| hsa-miR-3683 | hsa-miR-591 | hsa-miR-6770-5p | hsa-miR-3610 |
| hsa-miR-3687 | hsa-miR-329-3p | hsa-miR-1304-3p | hsa-miR-3611 |
| hsa-miR-3686 | hsa-miR-592 | hsa-miR-6859-3p | hsa-miR-3612 |
| hsa-miR-3685 | hsa-miR-595 | hsa-miR-628-5p | hsa-miR-593-5p |
| hsa-miR-3684 | hsa-miR-3184-3p | hsa-miR-4728-3p | hsa-miR-3615 |
| hsa-miR-1208 | hsa-miR-599 | hsa-miR-518f-5p | hsa-miR-3618 |
| hsa-miR-6776-5p | hsa-miR-6735-5p | hsa-miR-98-3p | hsa-miR-4804-3p |
| hsa-miR-1205 | hsa-miR-4440 | hsa-miR-202-3p | hsa-miR-6809-5p |
| hsa-miR-1204 | hsa-miR-4441 | hsa-miR-6792-3p | hsa-miR-1287-5p |
| hsa-miR-1206 | hsa-miR-4443 | hsa-miR-412-3p | hsa-miR-6849-5p |
| hsa-miR-1200 | hsa-miR-4444 | hsa-miR-6825-3p | hsa-miR-551a |
| hsa-miR-1203 | hsa-miR-4448 | hsa-miR-5188 | hsa-miR-1293 |
| hsa-miR-4310 | hsa-miR-759 | hsa-miR-4468 | hsa-miR-1290 |
| hsa-miR-4311 | hsa-miR-548aq-3p | hsa-miR-1199-3p | hsa-miR-26a-2-3p |
| hsa-miR-4312 | hsa-miR-578 | hsa-miR-3139 | hsa-miR-1297 |
| hsa-miR-4313 | hsa-miR-577 | hsa-miR-4464 | hsa-miR-1294 |
| hsa-miR-4314 | hsa-miR-6876-3p | hsa-miR-6734-3p | hsa-miR-190a-3p |

TABLE 14-5

| | | | |
|---|---|---|---|
| hsa-miR-504-3p | hsa-miR-655-3p | hsa-miR-4462 | hsa-miR-374b-3p |
| hsa-miR-4316 | hsa-miR-4733-5p | hsa-miR-4460 | hsa-miR-363-5p |
| hsa-miR-4317 | hsa-miR-4691-5p | hsa-miR-4461 | hsa-miR-4800-3p |
| hsa-miR-4318 | hsa-miR-5003-3p | hsa-miR-4669 | hsa-miR-372-5p |
| hsa-miR-4319 | hsa-miR-5187-5p | hsa-miR-513a-3p | hsa-miR-4769-3p |
| hsa-miR-6870-3p | hsa-miR-2278 | hsa-miR-548az-5p | hsa-miR-2053 |
| hsa-miR-548e-3p | hsa-miR-4671-5p | hsa-miR-4660 | hsa-miR-2052 |
| hsa-miR-891a-5p | hsa-miR-6752-3p | hsa-miR-4663 | hsa-miR-4670-5p |
| hsa-miR-3934-5p | hsa-miR-3682-5p | hsa-miR-559 | hsa-miR-2054 |
| hsa-miR-3606-3p | hsa-miR-301a-5p | hsa-miR-558 | hsa-miR-523-3p |
| hsa-miR-6788-5p | hsa-miR-3130-5p | hsa-miR-548aq-5p | hsa-miR-6770-3p |
| hsa-miR-218-5p | hsa-miR-519a-3p | hsa-miR-1185-5p | hsa-miR-6744-5p |
| hsa-miR-877-3p | hsa-miR-4713-3p | hsa-miR-554 | hsa-miR-6845-3p |
| hsa-miR-3688-3p | hsa-miR-4640-3p | hsa-miR-524-5p | hsa-miR-511-3p |
| hsa-miR-548g-3p | hsa-miR-410-5p | hsa-miR-553 | hsa-miR-651-3p |
| hsa-miR-598-3p | hsa-miR-3681-5p | hsa-miR-3621 | hsa-miR-518f-3p |
| hsa-miR-152-3p | hsa-miR-3680-3p | hsa-miR-9-3p | hsa-miR-4699-3p |
| hsa-miR-7162-3p | hsa-miR-4793-3p | hsa-miR-4733-3p | hsa-miR-1295b-5p |
| hsa-miR-664a-5p | hsa-miR-4524b-5p | hsa-miR-891b | hsa-miR-30b-3p |
| hsa-miR-874-3p | hsa-miR-24-1-5p | hsa-miR-676-5p | hsa-miR-7151-3p |
| hsa-miR-4765 | hsa-miR-6816-3p | hsa-miR-6834-5p | hsa-miR-1909-5p |
| hsa-miR-4767 | hsa-miR-1245b-5p | hsa-miR-4764-5p | hsa-miR-4487 |
| hsa-miR-3622b-5p | hsa-miR-302d-5p | hsa-miR-490-5p | hsa-miR-4480 |
| hsa-miR-6811-5p | hsa-miR-508-3p | hsa-miR-3682-3p | hsa-miR-876-5p |
| hsa-miR-499b-5p | hsa-miR-3692-3p | hsa-miR-608 | hsa-miR-4483 |
| hsa-miR-4772-3p | hsa-miR-8082 | hsa-miR-5087 | hsa-miR-27b-5p |
| hsa-miR-212-5p | hsa-miR-6499-5p | hsa-miR-4723-3p | hsa-miR-4489 |
| hsa-miR-518c-3p | hsa-miR-6810-5p | hsa-miR-7846-3p | hsa-miR-6780b-3p |
| hsa-miR-422a | hsa-miR-449b-3p | hsa-miR-4677-3p | hsa-miR-4648 |
| hsa-miR-548c-3p | hsa-miR-185-3p | hsa-miR-6755-5p | hsa-miR-513a-5p |
| hsa-miR-3679-3p | hsa-miR-6512-5p | hsa-miR-1537-5p | hsa-miR-6728-5p |
| hsa-miR-6818-3p | hsa-miR-6758-3p | hsa-miR-3680-5p | hsa-miR-4642 |
| hsa-miR-6754-5p | hsa-miR-6513-3p | hsa-miR-1303 | hsa-miR-6516-3p |
| hsa-miR-6864-3p | hsa-miR-4649-3p | hsa-miR-95-5p | hsa-miR-3150a-5p |

TABLE 14-6

| | | | |
|---|---|---|---|
| hsa-miR-129-5p | hsa-miR-6813-5p | hsa-miR-3119 | hsa-miR-4641 |
| hsa-miR-6823-3p | hsa-miR-6812-3p | hsa-miR-3118 | hsa-miR-4647 |
| hsa-miR-1468-5p | hsa-miR-5589-3p | hsa-miR-3116 | hsa-miR-4644 |
| hsa-miR-580-5p | hsa-miR-374c-3p | hsa-miR-3115 | hsa-miR-3186-5p |
| hsa-miR-548ak | hsa-miR-1263 | hsa-miR-412-5p | hsa-miR-6746-3p |
| hsa-miR-4999-5p | hsa-miR-1262 | hsa-miR-889-3p | hsa-miR-5009-5p |
| hsa-miR-548an | hsa-miR-1261 | hsa-miR-6748-3p | hsa-miR-6851-3p |
| hsa-miR-548al | hsa-miR-1267 | hsa-miR-4664-5p | hsa-miR-203b-3p |
| hsa-miR-548ac | hsa-miR-1265 | hsa-miR-490-3p | hsa-miR-5008-3p |
| hsa-miR-548ab | hsa-miR-1264 | hsa-miR-6760-3p | hsa-miR-152-5p |
| hsa-miR-548ag | hsa-miR-802 | hsa-miR-6499-3p | hsa-miR-7843-3p |
| hsa-miR-552-3p | hsa-miR-6742-3p | hsa-miR-517c-3p | hsa-miR-6818-5p |
| hsa-miR-548ax | hsa-miR-3934-3p | hsa-miR-376b-5p | hsa-miR-199b-5p |
| hsa-miR-488-5p | hsa-miR-3177-3p | hsa-miR-5011-5p | hsa-miR-622 |
| hsa-miR-4633-3p | hsa-miR-1911-5p | hsa-miR-3117-3p | hsa-miR-621 |
| hsa-miR-450b-3p | hsa-miR-1185-2-3p | hsa-miR-6810-3p | hsa-miR-620 |
| hsa-miR-548aw | hsa-miR-6788-3p | hsa-miR-340-3p | hsa-miR-1255b-2-3p |
| hsa-miR-216b-5p | hsa-miR-877-5p | hsa-miR-4738-3p | hsa-miR-626 |
| hsa-miR-4540 | hsa-miR-4727-5p | hsa-miR-6513-5p | hsa-miR-1296-5p |
| hsa-miR-4433b-5p | hsa-miR-489-5p | hsa-miR-6740-3p | hsa-miR-4693-3p |
| hsa-miR-1250-5p | hsa-miR-3688-5p | hsa-miR-548at-3p | hsa-miR-448 |
| hsa-miR-4659b-5p | hsa-miR-4680-5p | hsa-miR-4774-5p | hsa-miR-5000-3p |
| hsa-miR-6775-3p | hsa-miR-505-5p | hsa-miR-1243 | hsa-miR-4481 |
| hsa-miR-545-5p | hsa-miR-6871-3p | hsa-miR-1244 | hsa-miR-6855-3p |
| hsa-miR-4788 | hsa-miR-380-3p | hsa-miR-3927-5p | hsa-miR-1298-3p |
| hsa-miR-4785 | hsa-miR-8071 | hsa-miR-1248 | hsa-miR-3127-3p |
| hsa-miR-4784 | hsa-miR-5010-5p | hsa-miR-6852-5p | hsa-miR-561-3p |
| hsa-miR-4780 | hsa-miR-6892-3p | hsa-miR-548ah-3p | hsa-miR-3134 |
| hsa-miR-3675-3p | hsa-miR-4643 | hsa-miR-501-3p | hsa-miR-3137 |
| hsa-miR-4717-3p | hsa-miR-499b-3p | hsa-miR-520a-5p | hsa-miR-3133 |
| hsa-miR-6835-5p | hsa-miR-6872-3p | hsa-miR-4766-5p | hsa-miR-3132 |
| hsa-miR-6828-5p | hsa-miR-200a-3p | hsa-miR-3177-5p | hsa-miR-513c-5p |
| hsa-miR-5699-3p | hsa-miR-181d-3p | hsa-miR-1180-3p | hsa-miR-7848-3p |

TABLE 14-7

| | | | |
|---|---|---|---|
| hsa-miR-6720-5p | hsa-miR-3646 | hsa-miR-4659a-5p | hsa-miR-3138 |
| hsa-miR-4722-3p | hsa-miR-8070 | hsa-miR-489-3p | hsa-miR-5194 |
| hsa-miR-4740-5p | hsa-miR-6767-5p | hsa-miR-382-3p | hsa-miR-5191 |
| hsa-miR-191-3p | hsa-miR-1269b | hsa-miR-4260 | hsa-miR-5190 |
| hsa-miR-8068 | hsa-miR-1269a | hsa-miR-4261 | hsa-miR-5193 |
| hsa-miR-604 | hsa-miR-520f-3p | hsa-miR-570-3p | hsa-miR-5192 |
| hsa-miR-4779 | hsa-miR-4694-3p | hsa-miR-4263 | hsa-miR-4524a-3p |
| hsa-miR-5004-5p | hsa-miR-525-3p | hsa-miR-4264 | hsa-miR-5011-3p |
| hsa-miR-1225-3p | hsa-miR-3671 | hsa-miR-4265 | hsa-miR-892c-5p |
| hsa-miR-4435 | hsa-miR-548ap-5p | hsa-miR-4266 | hsa-miR-4776-5p |
| hsa-miR-4434 | hsa-miR-302a-3p | hsa-miR-4267 | hsa-miR-7107-3p |
| hsa-miR-4437 | hsa-miR-4666b | hsa-miR-4268 | hsa-miR-1247-5p |
| hsa-miR-3124-5p | hsa-miR-875-5p | hsa-miR-154-3p | hsa-miR-3190-5p |
| hsa-miR-4431 | hsa-miR-4799-3p | hsa-miR-6871-5p | hsa-miR-129-2-3p |
| hsa-miR-4430 | hsa-miR-550a-3-5p | hsa-miR-6079 | hsa-miR-219b-5p |
| hsa-miR-4724-5p | hsa-miR-6729-3p | hsa-miR-6827-3p | hsa-miR-6766-3p |
| hsa-miR-4432 | hsa-miR-6887-3p | hsa-miR-494-3p | hsa-miR-6740-5p |
| hsa-miR-601 | hsa-miR-6823-5p | hsa-miR-6892-5p | hsa-miR-30c-2-3p |
| hsa-miR-1251-5p | hsa-miR-302b-3p | hsa-miR-1910-5p | hsa-miR-6776-3p |
| hsa-miR-6716-5p | hsa-miR-7156-5p | hsa-miR-1233-3p | hsa-miR-548at-5p |
| hsa-miR-600 | hsa-miR-208b-3p | hsa-miR-585-5p | hsa-miR-4801 |
| hsa-miR-346 | hsa-miR-153-5p | hsa-miR-3666 | hsa-miR-504-5p |
| hsa-miR-8062 | hsa-miR-8075 | hsa-miR-3661 | hsa-miR-668-5p |
| hsa-miR-3064-3p | hsa-miR-6730-3p | hsa-miR-3660 | hsa-miR-186-3p |
| hsa-miR-4668-3p | hsa-miR-5584-5p | hsa-miR-3662 | hsa-miR-653-5p |
| hsa-miR-4708-5p | hsa-miR-5585-3p | hsa-miR-6883-3p | hsa-miR-4712-3p |
| hsa-miR-377-5p | hsa-miR-4474-5p | hsa-miR-4469 | hsa-miR-3942-5p |
| hsa-miR-8061 | hsa-miR-7157-5p | hsa-miR-3668 | hsa-miR-5047 |
| hsa-miR-4282 | hsa-miR-6804-3p | hsa-miR-224-3p | hsa-miR-146b-3p |
| hsa-miR-4328 | hsa-miR-3120-5p | hsa-miR-5591-3p | hsa-miR-4697-3p |
| hsa-miR-4439 | hsa-miR-455-3p | hsa-miR-4743-3p | hsa-miR-3173-3p |
| hsa-miR-6889-3p | hsa-miR-3121-3p | hsa-miR-4465 | hsa-miR-1180-5p |
| hsa-miR-8084 | hsa-miR-4717-5p | hsa-miR-487b-5p | hsa-miR-7155-5p |

TABLE 14-8

| | | | |
|---|---|---|---|
| hsa-miR-4438 | hsa-miR-6504-5p | hsa-miR-6879-3p | hsa-miR-3916 |
| hsa-miR-1252-5p | hsa-miR-5699-5p | hsa-miR-875-3p | hsa-miR-3915 |
| hsa-miR-4796-5p | hsa-miR-149-5p | hsa-miR-4692 | hsa-miR-3914 |
| hsa-miR-4797-3p | hsa-miR-210-5p | hsa-miR-4696 | hsa-miR-3911 |
| hsa-miR-3678-5p | hsa-miR-6756-3p | hsa-miR-4698 | hsa-miR-3910 |
| hsa-miR-6836-5p | hsa-miR-580-3p | hsa-miR-5586-5p | hsa-miR-3919 |
| hsa-miR-548au-3p | hsa-miR-371b-5p | hsa-miR-6895-5p | hsa-miR-3918 |
| hsa-miR-6837-5p | hsa-miR-5089-5p | hsa-miR-19a-5p | hsa-miR-3191-5p |
| hsa-miR-1288-5p | hsa-miR-181c-3p | hsa-miR-4507 | hsa-miR-1193 |
| hsa-miR-3135a | hsa-miR-587 | hsa-miR-770-5p | hsa-miR-182-3p |
| hsa-miR-5007-3p | hsa-miR-583 | hsa-miR-7156-3p | hsa-miR-7162-5p |
| hsa-miR-33a-3p | hsa-miR-5088-3p | hsa-miR-6840-5p | hsa-miR-1197 |
| hsa-miR-548as-5p | hsa-miR-581 | hsa-miR-6873-5p | hsa-miR-874-5p |
| hsa-miR-1258 | hsa-miR-5581-3p | hsa-miR-548j-5p | hsa-miR-4790-5p |
| hsa-miR-433-5p | hsa-miR-6722-5p | hsa-miR-6715a-3p | hsa-miR-6793-5p |
| hsa-miR-432-3p | hsa-miR-6806-3p | hsa-miR-3605-3p | hsa-miR-3667-5p |
| hsa-miR-3162-3p | hsa-miR-4453 | hsa-miR-6817-3p | hsa-miR-1273h-5p |
| hsa-miR-211-5p | hsa-miR-4452 | hsa-miR-5585-5p | hsa-miR-1283 |
| hsa-miR-3157-3p | hsa-miR-4451 | hsa-miR-3654 | hsa-miR-548av-5p |
| hsa-miR-369-3p | hsa-miR-4450 | hsa-miR-4803 | hsa-miR-1910-3p |
| hsa-miR-4745-3p | hsa-miR-4457 | hsa-miR-6814-3p | hsa-miR-517-5p |
| hsa-miR-6503-3p | hsa-miR-4456 | hsa-miR-3121-5p | hsa-miR-585-3p |
| hsa-miR-6723-5p | hsa-miR-4455 | hsa-miR-4749-3p | hsa-miR-6877-3p |
| hsa-miR-3692-5p | hsa-miR-4458 | hsa-miR-6511a-3p | hsa-miR-3609 |
| hsa-miR-939-3p | hsa-miR-568 | hsa-miR-3650 | hsa-miR-548o-3p |
| hsa-miR-6874-5p | hsa-miR-563 | hsa-miR-323b-3p | hsa-miR-6890-5p |
| hsa-miR-4666a-3p | hsa-miR-6165 | hsa-miR-4715-5p | hsa-miR-1284 |
| hsa-miR-519e-3p | hsa-miR-377-3p | hsa-miR-5088-5p | hsa-miR-1286 |
| hsa-miR-378b | hsa-miR-503-3p | hsa-miR-4482-3p | hsa-miR-1281 |
| hsa-miR-378c | hsa-miR-1252-3p | hsa-miR-4471 | hsa-miR-200c-5p |
| hsa-miR-6830-3p | hsa-miR-1282 | hsa-miR-1289 | hsa-miR-192-3p |

(15)

Classifiers Developed Using Various Sets of Markers

In some embodiments, about or at least 1, 2, 3, 4, 5, 6, 7, 8, 9, 10, 11, 12, 13, 14, or 15 non-coding RNAs that are selected from Table 2 (with or without any non-coding RNAs from the core set) can be used in a classifier. In some embodiments, the non-coding RNAs in Table 1 and Table 2 can be combined, and about or at least 1, 2, 3, 4, 5, 6, 7, 8, 9, 10, 20, or 25 miRNAs from the two combined tables can be used in a classifier.

In some embodiments, about or at least 1, 2, 3, 4, 5, 6, 7, 8, 9, 10, 11, 12, 13, 14, 15, 16, 17, 18, 19, 20, 21, 22, 23, 24, or 25 non-coding RNAs selected from Table 3 (with or without any non-coding RNAs from Set 2) can be used in a classifier. In some embodiments, the non-coding RNAs in Tables 1-3 can be combined, and about or at least 1, 2, 3, 4, 5, 6, 7, 8, 9, 10, 20, 30, 40, or 50 miRNAs from the three combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 4 (with or without any non-coding RNAs from Set 3) can be used in a classifier. In some embodiments, the non-coding RNAs in Tables 1-4 can be combined, and about or at least 1, 2, 3, 4, 5, 6, 7, 8, 9, 10, 20, 30, 40, 50, 60, 70, 80, 90, or 100 miRNAs from the four combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 5 (with or without any non-coding RNAs from Set 4) can be used in a classifier. In some embodiments, the non-coding RNAs in Tables 1-5 can be combined, and about or at least 1, 2, 3, 4, 5, 6, 7, 8, 9, 10, 20, 30, 40, 50, 60, 70, 80, 90, 100, 110, 120, 130, 140, or 150 miRNAs from the five combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 6 (with or without any non-coding RNAs from Set 5) can be used in a classifier. In some embodiments, the non-coding RNAs in Tables 1-6 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, or all miRNAs from the six combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 7 (with or without any non-coding RNAs from Set 6) can be used in a classifier. In some embodiments, the non-coding RNAs in Tables 1-7 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, or 250 miRNAs from the seven combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, or 50 non-coding RNAs selected from Table 8 (with or without any non-coding RNAs from Set 7) can be used in a classifier. In some embodiments, the non-coding RNAs in Tables 1-8 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290 or all miRNAs from the eight combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 non-coding RNAs selected from Table 9 (with or without any non-coding RNAs from Set 8) can be used in a classifier. In some embodiments, the non-coding RNAs in Tables 1-9 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350 or 400 miRNAs from the nine combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 non-coding RNAs selected from Table 10 (with or without any non-coding RNAs from Set 9) can be used in a classifier. In some embodiments, the non-coding RNAs in Tables 1-10 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, or 500 miRNAs from the ten combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 non-coding RNAs selected from Table 11 (with or without any non-coding RNAs from Set 10) can be used in a classifier. In some embodiments, the non-coding RNAs in Tables 1-11 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, or 600 miRNAs from the eleven combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, or 200 non-coding RNAs selected from Table 12 (with or without any non-coding RNAs from Set 11) can be used in a classifier. In some embodiments, the non-coding RNAs in Tables 1-12 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, or 800 miRNAs from the twelve combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, or 200 non-coding RNAs selected from Table 13 (with or without any non-coding RNAs from Set 12) can be used in a classifier. In some embodiments, the non-coding RNAs in Tables 1-13 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 miRNAs from the 13 combined tables can be used in a classifier.

In some embodiments, about or at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, or 1004 non-coding RNAs selected from Table 14 (with or without any non-coding RNAs from Set 13) can be used in a classifier. In some embodiments, the non-coding RNAs in Tables 1-14 can be combined, and about or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1100, 1200, 1300, 1400, 1500, 1600, 1700, 1800, 1900, 2000, or 2004 miRNAs from the 14 combined tables can be used in a classifier.

In some embodiments, the non-coding RNAs that are selected for use in a classifier do not include one or more miRNAs selected from the group consisting of hsa-miR-1246, hsa-miR-1307-3p, hsa-miR-4634, hsa-miR-6861-5p and hsa-miR-6875-5p.

Classifiers

Referring to FIG. 1, classifiers are generated via data processing system 18 by applying one or more mathematical models to data representative of the level of non-coding RNAs (e.g., microRNAs) across a population encompassing both subjects who have a breast disease and subjects who do not have a breast disease. In some embodiments, classifiers are generated by applying one or more mathematical models to data representative of the level of non-coding RNAs (e.g., microRNAs) across a population encompassing both subjects who have breast cancer and subjects who do not have breast cancer (e.g., healthy subjects, subjects who have non-malignant breast diseases, or a cancer that is not breast cancer).

The mathematical model can be any mathematical model as described herein. In these embodiments, data processing system 18 generates the classifier by applying the mathematical model with a set of biomarkers to the training dataset to determine values for parameters for the mathematical models. Generally, the training dataset includes data representing levels of non-coding RNAs (e.g., microRNAs) in samples obtained from individuals of a training population (e.g., individuals who have breast cancer, healthy individuals, individuals who have non-malignant breast diseases, or individuals who have a cancer that is not breast cancer). As described above, data processing system 18 generates and trains a classifier for each set of non-coding RNAs. The classifier, which includes the mathematical model and the determined values of logistic regression equation coefficients and logistic regression equation constants, can be used to determine a likelihood score indicating a probability that a subject has breast cancer or a benign breast disease, or a probability that the subject is healthy. Data processing system 18 then applies one or more of these generated classifiers to data specifying the level of one or more non-coding RNAs of the set of non-coding RNAs in a sample from the test subject, to determine a likelihood score indicating a probability that the test subject has breast cancer or a benign breast disease, or a probability that the test subject is healthy.

In some embodiments, the set of biomarkers (e.g., non-coding RNAs) is selected based on the rule disclosed herein. In other embodiments, an individual non-coding RNA is selected based on the p value as a measure of the likelihood that the non-coding RNA can distinguish between the two phenotypic trait subgroups (e.g., subjects who have breast cancer vs. subjects who do not have breast cancer). Thus, in some embodiments, biomarkers are chosen to test in combination by input into a model wherein the p value of each biomarker is less than 0.2, less than 0.1, less than 0.5, less than 0.1, less than 0.05, less than 0.01, less than 0.005, less than 0.001, less than 0.0005, less than 0.0001, less than 0.00005, less than 0.00001, less than 0.000005, less than 0.000001, etc.

Classifiers can be used alone or in combination with each other to create a stacking ensemble classifier for determining the probability that a test subject has breast cancer or a benign breast disease. One or more selected classifiers can be used to generate a stacking ensemble classifier. Thus, a stacking ensemble classifier can have about or at least 2, 3, 4, 5, 6, 7, 8, 9, or 10 levels of sub-classifiers. A sub-classifier at a higher level can be used to combine the results of two or more sub-classifier at a lower level. For example, some statistical techniques (e.g., normalization, imputation, or TSNE) can be applied to a dataset to transform the data. The first level of sub-classifiers (e.g., random forest, logistic regression, extra tree classifier, Support Vector Machine (SVM), K-nearest neighbors, multilayer perceptron (MLP), deep learning classifiers, neural network, feed forward neural network, convolutional neural network, recurrent neural network or gradient boosting decision trees) can be applied to the transformed data. The second level of sub-classifiers can be applied to the results of the first level of sub-classifiers and/or the transformed data. In some embodiments, additional levels of sub-classifiers can be included in the stacking ensemble classifier.

In some embodiments, the individuals of the training population used to derive the model are different from the individuals of a population used to test the model. As would be understood by a person skilled in the art, this allows a person skilled in the art to characterize an individual whose phenotypic trait characterization is unknown, for example, to determine a likelihood score indicating the probability of the individual's having breast cancer or a benign breast disease, or indicating the probability that the individual is healthy.

The data that is input into the mathematical model can be any data that is representative of the level of biomarkers (e.g., non-coding RNAs). Mathematical models useful in accordance with the disclosure include those using both supervised and unsupervised learning techniques. In one embodiment, the mathematical model chosen uses supervised learning in conjunction with a training population to evaluate each possible combination of biomarkers (e.g., non-coding RNAs). Various mathematical models can be used, for example, a regression model, a logistic regression model, a neural network, a clustering model, principal component analysis, nearest neighbor classifier analysis, linear discriminant analysis, quadratic discriminant analysis, a support vector machine, a decision tree, a genetic algorithm, classifier optimization using bagging, classifier optimization using boosting, classifier optimization using the Random Subspace Method, a projection pursuit, and genetic programming and weighted voting, etc.

Applying a mathematical model to the data will generate one or more classifiers. In some embodiments, multiple classifiers are created that are satisfactory for the given purpose (e.g., all have sufficient AUC and/or sensitivity and/or specificity). In some embodiments, a formula is generated that utilizes more than one classifier. For example, a formula can be generated that utilizes classifiers in series. Other possible combinations and weightings of classifiers would be understood and are encompassed herein.

A classifier can be evaluated for its ability to properly characterize each individual of a population (e.g., a training population or a validation population) using methods known to a person of ordinary skill in the art. Various statistical criteria can be used, for example, area under the curve (AUC), sensitivity and/or specificity. In one embodiment, the classifier is evaluated by cross validation, Leave One OUT Cross Validation (LOOCV), n-fold cross validation, and jackknife analysis. In another embodiment, each classifier is evaluated for its ability to properly characterize those individuals is a population that was not used to generate the classifier.

In some embodiments, a confusion matrix can be generated to evaluate a classifier. In statistics, a confusion matrix is a specific table layout that allows visualization of the performance of an algorithm. The confusion matrix reports the number of false positives, false negatives, true positives, and true negatives, which allows more detailed analysis than mere proportion of correct classifications.

From the confusion matrix, accuracy, specificity, sensitivity, precision (positive predictive value), negative predictive value and F1-Score can be calculated. (See Table 16). In some embodiments, the classifier has an outstanding performance with a value for accuracy, specificity, sensitivity, precision, negative predictive value, and/or F1-score that is about or at least 0.99, 0.98, 0.97, 0.96, 0.95, 0.94, 0.93, 0.92, 0.91, 0.9, 0.85, or 0.8.

In some embodiments, the method used to evaluate the classifier for its ability to properly characterize each individual of the training population is a method that evaluates the classifier's sensitivity (true positive fraction) and 1—specificity (true negative fraction). In one embodiment, the method used to test the classifier is a Receiver Operating Characteristic (ROC), which provides several parameters to evaluate both the sensitivity and the specificity of the result of the equation generated. In one embodiment, the ROC area (area under the curve) is used to evaluate the equations. A ROC area greater than 0.5, 0.6, 0.7, 0.8, or 0.9 is preferred. A perfect ROC area score of 1.0 is indicative of both 100% sensitivity and 100% specificity. In some embodiments, classifiers are selected on the basis of the various scores (e.g., accuracy, specificity, sensitivity, precision (positive predictive value), negative predictive value, F1-Score, or AUC). In an example, the scoring system used is a ROC curve score determined by the area under the ROC curve. In this example, classifiers with scores of greater than 0.95, 0.9, 0.85, 0.8, 0.7, 0.65, 0.6, 0.55, or 0.5 are chosen. In other embodiments, where specificity is important to the use of the classifier, a sensitivity threshold can be set, and classifiers ranked on the basis of the specificity are chosen. For example, classifiers with a cutoff for specificity of greater than 0.99, 0.98, 0.97, 0.96, 0.95, 0.94, 0.93, 0.92, 0.91, 0.9, 0.85, 0.8, 0.7, 0.65, 0.6, 0.55, 0.5 or 0.45 can be chosen. Similarly, the specificity threshold can be set, and classifiers ranked on the basis of sensitivity (e.g., greater than 0.99, 0.98, 0.97, 0.96, 0.95, 0.94, 0.93, 0.92, 0.91, 0.9, 0.85, 0.8, 0.7, 0.65, 0.6, 0.55, 0.5 or 0.45) can be chosen. Thus, in some embodiments, only the top ten ranking classifiers, the top twenty ranking classifiers, or the top one hundred ranking classifiers are selected. The ROC curve can be calculated by various statistical tools, including but not limited to Statistical Analysis System (SAS), R, and CORExpress® statistical analysis software.

As would be understood by a person of ordinary skill in the art, the utility of the combinations and classifiers determined by a mathematical model will depend upon some characteristics (e.g., race, age group, gender, medical history) of the population used to generate the data for input into the model. One can select the individually identified biomarkers (e.g., non-coding RNAs such as miRNAs) or subsets of the individually identified biomarkers (e.g., non-coding RNAs such as miRNAs), and test all possible combinations of the selected biomarkers to identify a useful combinations of biomarkers.

Populations for Input into the Mathematical Models

Populations used for input should be chosen so as to result in a statistically significant classifier. In some embodiments, the reference or training population includes between 50 and 100 subjects. In another embodiment, the reference population includes between 100 and 500 subjects. In still other embodiments, the reference population includes two or more populations, each including between 50 and 100, between 100 and 500, between 500 and 1000, between 1000 and 1500, between 1500 and 2000, between 2000 and 2500, or more than 3000 subjects. The reference population includes two or more subpopulations. In one embodiment, the phenotypic trait characteristics of the two or more subpopulations are similar but for the phenotypic trait that is under investigation, for example, having a breast disease, having breast cancer, or having a benign breast disease. In some embodiments, the subpopulations are of roughly equivalent numbers. The present methods do not require using data from every member of a population, but instead may rely on data from a subset of a population in question.

In some embodiments, a test population (or validation population) that is comprised of individuals who have breast cancer and individuals who do not have breast cancer is used to evaluate a classifier for its ability to properly classify each individual.

Data for Input into the Mathematical Models

Data for input into the mathematical models are data representative of the respective levels of biomarkers (e.g., expression levels of non-coding RNAs). The non-coding RNAs include, but are not limited to, miRNAs, siRNAs, piRNAs, snoRNAs, snRNAs, exRNAs, scaRNAs and long ncRNAs, and particularly miRNAs.

A dataset can be used to generate a classifier. The “dataset,” in the context of a dataset to be applied to a classifier, can include data representing levels of each biomarker for each individual. However, in some embodiments, the dataset does not need to include data for each biomarker of each individual. For example, the dataset includes data representing levels of each biomarker for fewer than all of the individuals (e.g., 99%, 95%, 90%, 85%, 80%, 75%, 70% or fewer) and can still be useful for purposes of generating a classifier. In some embodiments, an imputed value for a given biomarker (e.g., median of all known values for the biomarker in the dataset) can be used to replace a missing value for that biomarker in the data.

In some embodiments, normalization can be performed before applying a mathematical model to the dataset. Normalization refers to a process of adjusting values measured on different scales to a notionally common scale. In some embodiments, normalization can align distributions of values to a normal distribution.

In some embodiments, dimensionality reduction on the data can be performed before applying a mathematical mode to the data to generate a classifier. Various dimensionality reduction techniques are known in the art, including, e.g., principal component analysis, non-negative matrix factorization, or t_Distributed Stochastic Neighbor Embedding (TSNE). The TSNE technique models each high-dimensional object by a two- or three-dimensional point in such a way that similar objects are modeled by nearby points and dissimilar objects are modeled by distant points with high probability. The t-SNE algorithm comprises two main stages. First, t-SNE constructs a probability distribution over pairs of high-dimensional objects in such a way that similar objects have a high probability of being picked while dissimilar points have an extremely small probability of being picked. Second, t-SNE defines a similar probability distribution over the points in the low-dimensional map, and it minimizes the Kullback-Leibler divergence between the two distributions with respect to the locations of the points in the map. It is a nonlinear dimensionality reduction technique well-suited for embedding high-dimensional data for visualization in a low-dimensional space of two or three dimensions.

Mathematic Models

Various types of mathematical models, including, e.g., random forest, logistic regression, extra tree classifier, Support Vector Machine (SVM), K-nearest neighbors, multilayer perceptron (MLP), deep learning classifiers, neural networks, feed forward neural networks, convolutional neural networks, recurrent neural networks or gradient boosting decision trees, can be used to construct classifiers useful to determine whether a subject is relatively likely to have breast cancer, a benign breast disease, or is likely to be a healthy individual.

In some embodiments, the mathematical model is random forest. Random forests or random decision forests are an ensemble learning method for classification, regression and other tasks that operates by constructing a multitude of decision trees at training time and outputting the class that is the mode of the classes (classification) or mean prediction (regression) of the individual trees. In some embodiments, the mathematical model is an extra tree classifier. The extra-tree approach finds an optimal cut-point for each one of the K randomly chosen features at each node, rather than uses bootstrap copies of the learning sample as used in random forest. It essentially consists of randomizing strongly both attribute and cut-point choice while splitting a tree node. In the extreme case, it builds totally randomized trees whose structures are independent of the output values of the learning sample. The strength of the randomization can be tuned to problem specifics by the appropriate choice of a parameter. Besides accuracy, the extra-tree approach is known for its computational efficiency. This method is described in detail in Geurts et al., “Extremely randomized trees.” Machine learning 63.1 (2006): 3-42.

In some embodiments, the mathematical model is a regression model, for example, a logistic regression model or a linear regression model. The regression model estimates the relationships among variables. It focuses on the relationship between a dependent variable and one or more independent variables (also known as predictors). The linear regression is a linear approach to modelling the relationship between a scalar response (or dependent variable) and one or more explanatory variables (or independent variables). In contrast, the logistic model (or logit model) usually uses a logistic function to model a binary dependent variable. It is well suited for modeling a binary dependent variable (e.g., a variable that has only two values, e.g., “having cancer” vs. “not having cancer”).

In some embodiments, the mathematical model is Support Vector Machine (SVM). SVM are supervised learning models with associated learning algorithms that analyze data used for classification and regression analysis. An SVM model is a representation of the examples as points in space, mapped so that the examples of the separate categories are divided by a clear gap that is as wide as possible. New examples are then mapped into that same space and predicted to belong to a category based on which side of the gap they fall. Given a set of training examples, each marked as belonging to one or the other of two categories, the SVM training algorithm usually builds a model that assigns new examples to one category or the other, making it a non-probabilistic binary linear classifier.

In some embodiments, the K-nearest neighbors algorithm can be used. The output of K-nearest neighbors algorithm is a class membership. An object is classified by a plurality vote of its neighbors, with the object being assigned to the class most common among its K nearest neighbors (K is a positive integer, typically small). In some cases, the K-nearest neighbors algorithm can be used to determine a predicted value of an object. This value is sometimes the average of the values of K nearest neighbors.

In some embodiments, the mathematical model is multilayer perceptron (MLP) (e.g., MLP 3 or MLP 4). A multilayer perceptron is a class of feedforward artificial neural network. A MLP consists of at least three layers of nodes: an input layer, a hidden layer, and an output layer. Except for the input nodes, each node is a neuron that uses a nonlinear activation function. MLP utilizes a supervised learning technique called backpropagation for training. Its multiple layers and non-linear activation distinguish MLP from a linear perceptron. It can distinguish data that is not linearly separable.

In some embodiments, the mathematical model involves deep learning. The deep learning refers to a family of machine learning methods based on artificial neural networks. These methods can progressively improve their ability to do tasks by considering examples, generally without task-specific programming. In some embodiments, the deep learning is based on feed forward neural network, convolutional neural network, or recurrent neural network.

The feedforward neural network is an artificial neural network wherein connections between the nodes do not form a cycle. In this network, the information moves in only one direction, forward, from the input nodes, through the hidden nodes (if any) and to the output nodes. There are no cycles or loops in the network. The simplest kind of neural network is a single-layer perceptron network, which consists of a single layer of output nodes; the inputs are fed directly to the outputs via a series of weights. The sum of the products of the weights and the inputs is calculated in each node, and if the value is above some threshold (typically 0) the neuron fires and takes the activated value (typically 1); otherwise it takes the deactivated value (typically −1). Neurons with this kind of activation function are also called artificial neurons or linear threshold units. A perceptron can be created using any values for the activated and deactivated states as long as the threshold value lies between the two. Perceptrons can be trained by a simple learning algorithm that is usually called the delta rule. It calculates the errors between calculated output and sample output data, and uses this to create an adjustment to the weights, thus implementing a form of gradient descent. Multilayer perceptron is typically a type of feedforward neural network. It has multiple layers of computational units, usually interconnected in a feed-forward way.

Convolutional neural networks are regularized versions of multilayer perceptrons. Multilayer perceptrons usually refer to fully connected networks, that is, each neuron in one layer is connected to all neurons in the next layer. The “fully-connectedness” of these networks make them prone to overfitting data. Typical ways of regularization includes adding some form of magnitude measurement of weights to the loss function. However, convolutional neural networks take a different approach towards regularization: they take advantage of the hierarchical pattern in data and assemble more complex patterns using smaller and simpler patterns. Therefore, on the scale of connectedness and complexity, convolutional neural networks are on the lower extreme.

Recurrent neural network (RNN) is a class of artificial neural network where connections between nodes form a directed graph along a temporal sequence. This allows it to exhibit temporal dynamic behavior. Unlike feedforward neural networks, RNNs can use their internal state (memory) to process sequences of inputs. This makes them applicable to tasks such as unsegmented, connected handwriting recognition or speech recognition. The term “recurrent neural network” is used indiscriminately to refer to two broad classes of networks with a similar general structure, where one is finite impulse and the other is infinite impulse. Both classes of networks exhibit temporal dynamic behavior. A finite impulse recurrent network is a directed acyclic graph that can be unrolled and replaced with a strictly feedforward neural network, while an infinite impulse recurrent network is a directed cyclic graph that cannot be unrolled. A detailed description of these neural networks can be found, e.g., in Schmidhuber, Jaurgen. “Deep learning in neural networks: An overview.” Neural networks 61 (2015): 85-117; and Zhang et al. “Shift-invariant pattern recognition neural network and its optical architecture.” Proceedings of annual conference of the Japan Society of Applied Physics. (1988): Vol. 88. No. 11.

In some embodiments, the mathematical model is gradient boosting. It is a prediction model in the form of an ensemble of weak prediction models. For example, if it is built upon decision trees, it is known as gradient boosting decision trees. Gradient boosting builds the model in a stage-wise fashion, and it generalizes them by allowing optimization of an arbitrary differentiable loss function.

Kits

The levels of biomarkers can be determined by using a kit. Such a kit can include materials and reagents required for obtaining an appropriate sample from a subject, or for measuring the levels of particular biomarkers (e.g., non-coding RNAs). In some embodiments, the kits include only those materials and reagents that would be required for obtaining and storing a sample from a subject. The sample is then shipped to a service center to determine the levels of biomarkers.

In some embodiments, the kits are Quantitative PCR (QPCR) kits. In other embodiments, the kits are nucleic acid arrays. In one embodiment, kits for measuring an RNA product includes materials and reagents that are necessary for measuring the expression of the RNA product. For example, a microarray or a QPCR kit may contain only those reagents and materials that are necessary for measuring the levels of a set of the presently disclosed miRNAs.

The kits may include instructions for performing the assay and methods for interpreting and analyzing the data resulting from the performance of the assay. The kits may also include hybridization reagents and/or reagents necessary for detecting a signal when a probe hybridizes to a target nucleic acid sequence.

### EXAMPLES

The following examples do not limit the scope of the present disclosure.

### Example 1: Overview

Serum samples were obtained from breast cancer patients and patients with benign breast diseases who were admitted or referred to the National Cancer Center Hospital (NCCH) between 2008 and 2014. The samples were collected by simple venipuncture from the cubital space, and the serum was isolated from whole peripheral blood by centrifuge. Breast cancer patients with the following characteristics were excluded: (i) administration of medication before the collection of serum; and (ii) simultaneous or previous diagnosis of advanced cancer in other organs.

Control serum samples were obtained from healthy individuals in three cohorts. The first cohort included volunteers aged over 60 years recruited from the Japanese company Toray Industries in 2013. The inclusion criteria for this cohort were no history of cancer and no hospitalization during the last 3 months. The second cohort included individuals whose serum samples were collected and stored by the National Center for Geriatrics and Gerontology (NCGG) Biobank between 2010 and 2012. The final cohort included female volunteers aged over 35 years who were recruited from the Yokohama Minoru Clinic in 2015, with the same criteria as those of the first cohort. Serum samples from prostate cancer patients were also included to identify the miRNA that are specific for breast cancer.

Total RNA was extracted from a 300 μl serum sample using 3D-Gene® RNA extraction reagent from a liquid sample kit (Toray Industries, Inc., Kanagawa, Japan). Comprehensive miRNA expression analysis was performed using a 3D-Gene® miRNA Labeling kit and a 3D-Gene® Human miRNA Oligo Chip (Toray Industries, Inc.), which was designed to detect 2555 miRNA sequences registered in miRBase release 20. A detailed description of the samples and measurements of the samples can be found, e.g., in Shimomura et al. “Novel combination of serum microRNA for detecting breast cancer in the early stage.” Cancer science 107.3 (2016): 326-334. The microarray data of this study are in agreement with the Minimum Information About a Microarray Experiment (MIAME) guidelines and are publicly available through the GEO database of National Center for Biotechnology Information (NCBI). The access number for the data in GEO database is GSE73002. The data can be located and downloaded by using the access number.

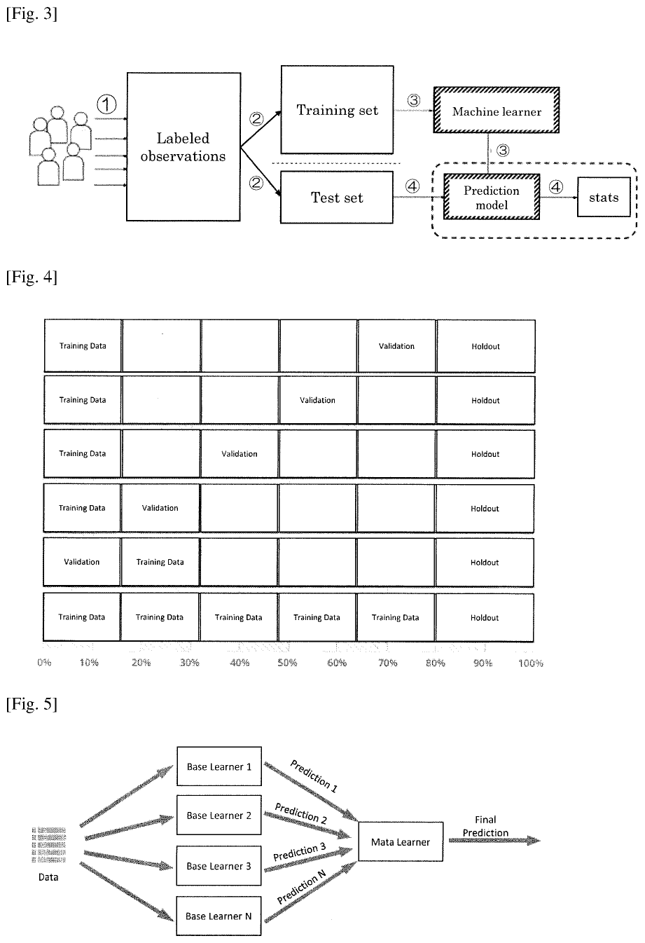

The dataset containing measurements (actual or imputed) for 2540 miRNAs in each of 4227 patient samples was generated. The dataset listed human patient samples in separate rows and miRNA probe measurements as columns (features). Each row had a unique label indicating the status of the patient, e.g., whether the patient is a healthy volunteer, has breast cancer, has prostate cancer, or has a benign breast disease. FIG. 3 is a schematic diagram showing the data modeling and machine learning workflow for predicting different status (e.g., individuals with breast cancer, individuals with a benign breast disease, or healthy individuals). The machine learning task was a supervised classification problem wherein a portion of the labelled dataset (training set) was used to train a classifier, and the classifier was then used to predict the status of the remaining subjects in the dataset (test or validation dataset). The below table shows the sample sizes for both the training set and the test dataset for each patient status:

TABLE 15

| | Sample Size (80% Training + |
|---|---|
| Status | 20% Test dataset = Total) |
| BRC (Breast cancer) | 1052 + 264 = 1316 |
| VOL (Healthy volunteers) | 2272 + 569 = 2841 |
| PR (Prostate Cancer) | 41 + 11 = 52 |
| BB (Benign Breast Disease) | 14 + 4 = 18 |

The data was pre-processed to ensure missing values in a given column were replaced by the median value for that column. The preprocessed dataset was randomly split into 80% training data and 20% testing data (the latter being the “hold out”). A classification model was then trained using optimized parameters and the training data. To prevent overfitting and bias, a 5-fold cross validation was employed. The training data were split into 5 groups (FIG. 4). 4 of the 5 groups were used to train the classifier, and the 5th group was used as a validation set for prediction. This process was repeated 5 times to select the best model on the overall training set. The simple model (SVM, or Support Vector Machine) and the competitor/alternate model (Stacking Ensemble Model) were selected.

(A) Simple Model (SVM, or Support Vector Machine)

The goal of the Support Vector Machine classifier was to find a hyperplane, i.e. a decision boundary that can classify the data points correctly in an N-dimensional space, where N is the number of features (N ranged from N_max: 2004 to N_min: 10, as features in the dataset were progressively sorted). This was achieved by finding the hyperplane/decision boundary with the maximum margin or distance between the classes of data points which correctly separates the classes of data points. This hyperplane was defined by support vectors that are data points close to the hyperplane and control its orientation and position. Squared_hinge (the square of the hinge loss) was used as a cost/loss function to maximize the margin between the data points and the hyperplane. A regularization parameter was added to the cost function to make the classifier less susceptible to outliers and improve its overall generalization. L1 norm is computationally less expensive, and is useful in feature selection as it ignores redundant features. Since the dataset had high dimensions, L1 normal was used for regularization. A C value of 50 was used, which ensures a larger margin hyperplane and results in less overfitting.

(B) Competitor/Alternative Model (Stacking Ensemble Model)

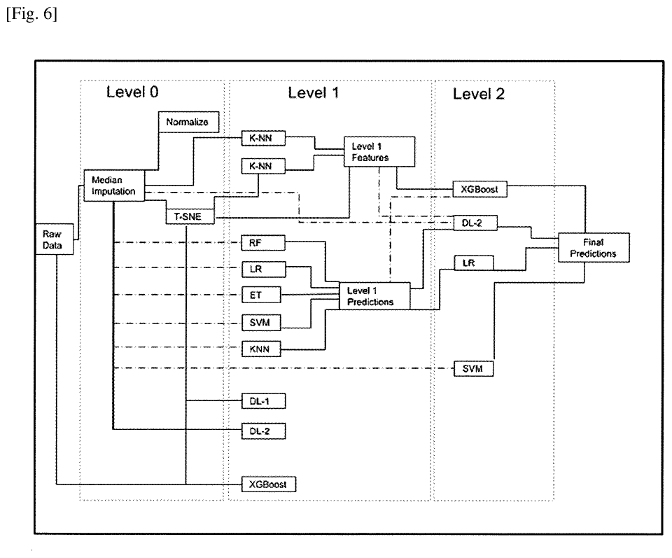

As shown in FIGS. 5 and 6, predictions from base models (Level 0/1: two or more) were combined and fed as inputs into a new model (Level 2: combiner model/meta learner). The predictions from the base models were used as inputs for the sequential layer and combined to form a new set of predictions.

Level 0

Level 0 learning included unsupervised feature extraction using three techniques: A) Median Imputation, B) Normalizer, and C) t_Distributed Stochastic Neighbor Embedding (TSNE) algorithm. Specifically, the raw data was initially transformed using A) Median Imputation, which replaces missing feature values using the median along the column. Next, the output of the pre-processed data from A) was transformed using B) Normalizer, where feature values were re-scaled within the range 0 and 1. The Normalizer worked as a row-wise operation. This was followed by C) TSNE, wherein the t_Distributed Stochastic Neighbor Embedding (TSNE) method was used for reducing the high dimensional data to low dimensions. The number of components=3 (dimensions) was kept.

The data formats from the above pre-processing techniques were then fed to base level (Level 1) classifiers.

Level 1

Data pre-processed via median imputation was fed as input individually to 1) Random Forest classifier, 2) Logistic Regression classifier, 3) Extra Trees Classifier, 4) Linear L1 SVM, and 5) K-Nearest Neighbours (KNN) classifier (K=2, 4, 8, 16, 32, 64, 128, or 256).

Raw data and t-SNE pre-processed data were also fed to the following classification algorithms: 6) Gradient Boosting Classifier (XGBoost) (Hyper parameters: objective=‘multi:softmax’, learning_rate=0.05, max_depth=5, n_estimators=1000, nthread=10, subsample=0.5, colsample_bytree=1.0).

Imputed data and t-SNE preprocessed data were also fed to the following classification algorithms: 7) Deep Learning Classifier 1 (DL-1); and 8) Deep Learning Classifier 2 (DL-2).

The Deep Learning Classifier 1 (DL-1) can be

a) a Multilayer Perceptron or a Feed Forward Neural Network with Parameters: i input layers, h hidden layers and o output size;

b) Convolutional Neural Network with Input Layer, Convolution Layer with shape of dimension 1 (m), shape of dimension 2 (n) and filters k and bias 1. Hence total parameters would be ((m*n)+1)*k), a Pool Layer and a fully connected layer which has ((n current layers*m previous layers)+1);

c) Recurrent Neural Network with Parameters: g layers in a unit, h hidden units, i size of input; or

d) any other appropriate deep learning classifier.

The Deep Learning Classifier 2 (DL-2) can be

a) a Multilayer Perceptron or a Feed Forward Neural Network with Parameters: i input layers, h hidden layers and o output size;

b) Convolutional Neural Network with Input Layer, Convolution Layer with shape of dimension 1 (m), shape of dimension 2 (n) and filters k and bias 1. Hence total parameters would be ((m*n)+1)*k), a Pool Layer and a fully connected layer which has ((n current layers*m previous layers)+1);

c) Recurrent Neural Network with Parameters: g layers in a unit, h hidden units, i size of input; or

d) any other appropriate deep learning classifier.

Imputed dataset from A) was also transformed using 9) the K-Nearest Neighbors algorithm (K=1, 2, or 4).

T-SNE preprocessed data was also transformed using 10) K-Nearest Neighbors algorithm (K=1, 2, or 4).

The following features and predictions probabilities were obtained from the output of Level 1 classification and transformation:

Level 1 features: t-SNE extracted features, KNN-transformed data with Median Imputed features, KNN-transformed data with t-SNE extracted features; and

Level 1 Predictions Probabilities: Output Predicted Probabilities from Level 1 Base Learners—Random Forest Classifier, Logistic Regression Classifier, Extra Trees Classifier, Linear SVM Classifier, Gradient Boosting Classifier, DL-1 and DL-2.

KNN Classifier with the best K values were all combined and fed to the Level 2 meta-learners.

Level 2

The following classifiers from the above outputs were trained to obtain the final predictions. Different inputs for different classification algorithms were used:

1) Logistic Regression: Input fed was Level 1 Prediction Probabilities;

2) Gradient Boosting: Input fed was Level 1 Prediction Probabilities+Level 1 Features

Hyperparameters (objective=‘multi:softmax’, learning_rate=0.1, max_depth=5, n_estimators=1000, nthread=10, subsample=0.9, colsample_bytree=0.7);

3) Deep learning classifier 2: Input fed was Median Imputed data+Level 1 Prediction Probabilities+Level 1 Features; and

4) SVM: Input fed was median Imputed data.

While any classifier from the above list can be chosen as the final meta-learner, gradient boosting was used to obtain the final predictions. The training method was designed such that the ensemble model trains the classifiers with random seeds (0, 1, 2, 3, 4). The prediction results of the Level 2 classifiers were aggregated by taking the geometric mean (one of the classifiers is chosen).

Prediction Model and Statistics

The best trained model was then used for prediction on the test dataset. The labels produced by the model (predictions) were compared with the original, true labels to generate a confusion matrix from where true positive (TP), false positive (FP), true negative (TN) and false negative (FN) values were obtained. An exemplary confusion matrix was then generated using the test dataset. These values were used to calculate the performance of the model, including metrics such as Accuracy, Specificity, Sensitivity (Recall), Precision (Positive Predictive Value), Negative Predictive Value and F1-Score (See Table 16). Weighted metrics were used for the calculation.

TABLE 16

| Metric | Formula |
|---|---|
| Accuracy | (TP + TN)/(TP + TN + FP + FN) |
| Precision | TP/(TP + FP) |
| Sensitivity | TP/(TP + FN) |
| F1-Score | 2 TP/(2TP + FP + FN) |
| Specificity | TN/(TN + FP) |
| NPV | TN/(TN + FN) |

### Example 2: Test Dataset Containing 2004 miRNA Measurements

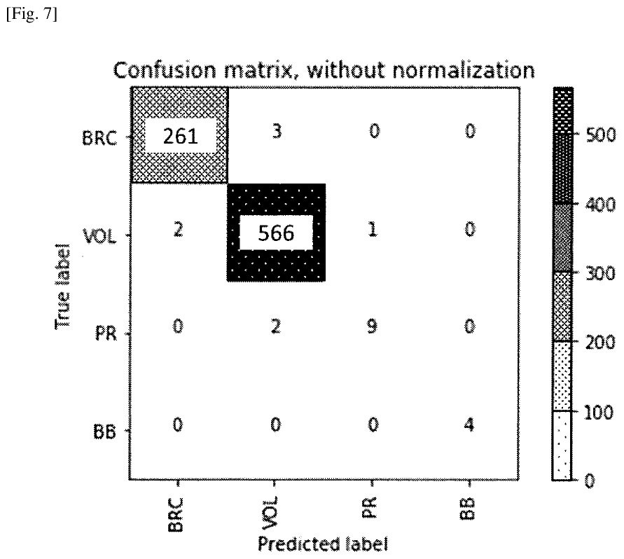

A dataset containing data representing expression levels of 2004 miRNA biomarkers (all miRNAs that are listed in Tables 1-14) for each subject was used to train and evaluate the classifier by the method as described in Example 1. (Where no expression level was obtained for a particular biomarker in a particular subject, a median value for that biomarker was substituted.) Using the results obtained from the test set of 848 subjects, a confusion matrix was generated (FIG. 7) and performance metrics were calculated (shown in Table 17 below).

The results showed that the classifier developed using these 2004 miRNA biomarkers had very high accuracy (>99%), very high sensitivity (>98%), and very high specificity (>99%) for breast cancer. The accuracy, sensitivity, and specificity for status of healthy individuals, of individuals with prostate cancer, and of individuals with benign breast diseases were also very high.

TABLE 17

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.994083 | 0.992395 | 0.988636 | 0.996558 | 0.994845 | 0.990512 | 264 |
| Cancer | | | | | | | |
| Vol (Healthy) | 0.990566 | 0.991243 | 0.994728 | 0.982079 | 0.98917 | 0.992982 | 569 |
| Prostate | 0.996441 | 0.9 | 0.818182 | 0.998798 | 0.997599 | 0.857143 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.99178 | 0.9904 | 0.9905 | 0.9868 | 0.9910 | 0.990 | 848 |

### Example 3: Test Dataset Containing 1000 miRNA Measurements

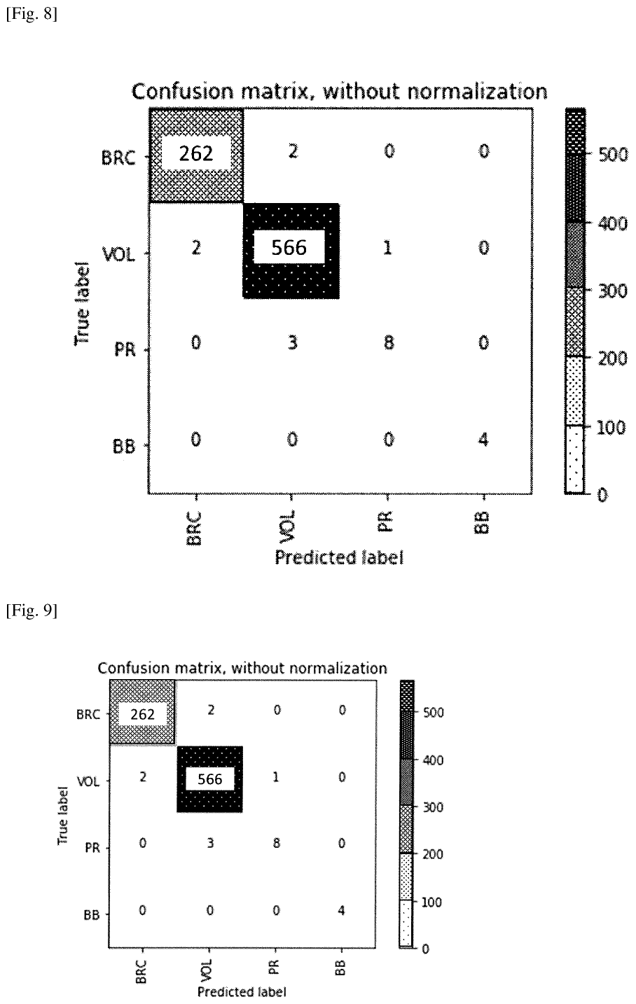

Based on the results from Example 2, a set of the 1000 miRNA with the highest prediction power was selected. A dataset containing measurements for the 1000 miRNAs (all of the miRNAs listed in Tables 1-13) for each subject was used to train and evaluate a classifier using these 1000 miRNA biomarkers as features. Using the results from the test set of 848 subjects, a confusion matrix was generated (FIG. 8) and performance metrics were calculated (shown in Table 18 below).

The results showed that the classifier developed using these 1000 miRNA biomarkers had very high accuracy (>99%), very high sensitivity (>99%), and very high specificity (>99%) for breast cancer. The accuracy, sensitivity, and specificity for status of healthy individuals, of individuals with prostate cancer, and of individuals with benign breast diseases were also very high.

TABLE 18

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.995261 | 0.992424 | 0.992424 | 0.996552 | 0.996552 | 0.992424 | 264 |
| Cancer | | | | | | | |
| Vol (Healthy) | 0.990566 | 0.991243 | 0.994728 | 0.982079 | 0.98917 | 0.992982 | 569 |
| Prostate | 0.995261 | 0.888889 | 0.727273 | 0.9988 | 0.996407 | 0.8 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9921 | 0.9903 | 0.9905 | 0.9868 | 0.9916 | 0.9903 | 848 |

### Example 4: Test Dataset Containing 800 miRNA Measurements

Based on the results from Example 3, a set of the 800 miRNA with the highest prediction power was selected from the 1000 miRNA biomarker set. A dataset containing measurements for the 800 miRNAs (all of the miRNAs listed in Tables 1-12) for each subject was used to train and evaluate a classifier using these 800 miRNA biomarkers as features. Using the results from the test set of 848 subjects, a confusion matrix was generated (FIG. 9) and performance metrics were calculated (shown in Table 19 below).

The results showed that the classifier developed using these 800 miRNA biomarkers had very high accuracy (>99%), very high sensitivity (>99%), and very high specificity (>99%) for breast cancer. The accuracy, sensitivity, and specificity for status of healthy individuals, of individuals with prostate cancer, and individuals with benign breast diseases were also very high.

TABLE 19

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9952 | 0.9924 | 0.9924 | 0.9965 | 0.9965 | 0.9924 | 264 |
| Cancer | | | | | | | |
| Vol (Healthy) | 0.9905 | 0.9912 | 0.9947 | 0.9820 | 0.9891 | 0.9929 | 569 |
| Prostate | 0.9952 | 0.8888 | 0.7272 | 0.9988 | 0.9964 | 0.8 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9921 | 0.99032 | 0.9905 | 0.9868 | 0.9916 | 0.9903 | 848 |

### Example 5: Test Dataset Containing 600 miRNA Measurements

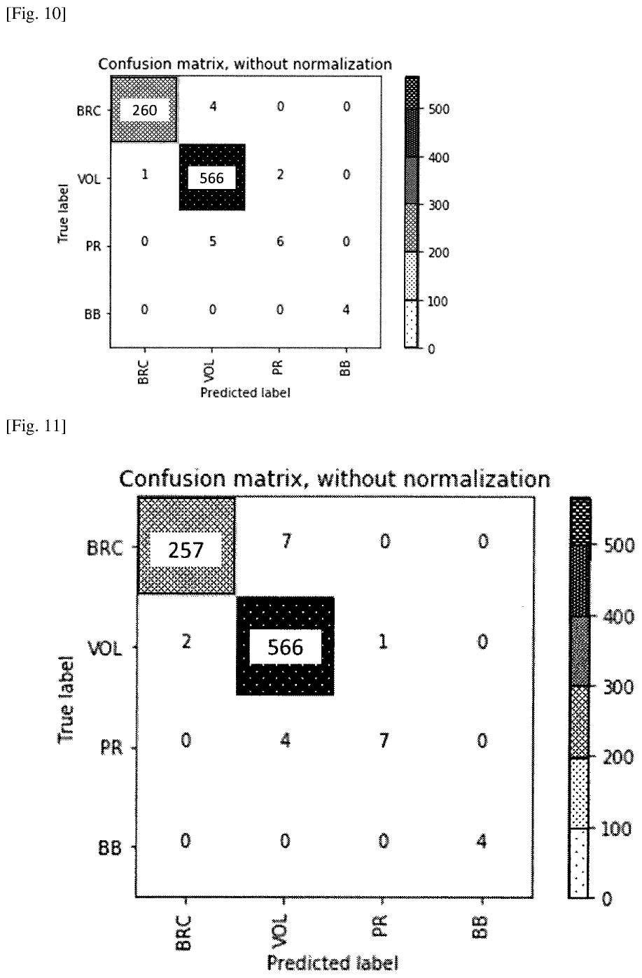

Based on the results from Example 4, a set of the 600 miRNA with the highest prediction power was selected from the 800 miRNA biomarker set. A dataset containing measurements for the 600 miRNAs (all of the miRNAs listed in Tables 1-11) for each subject was used to train and evaluate a classifier using these 600 miRNA biomarkers as features. Using the results from the test set of 848 subjects, a confusion matrix was generated (FIG. 10) and performance metrics were calculated (shown in Table 20 below).

The results showed that the classifier developed using these 600 miRNA biomarkers had very high accuracy (>99%), very high sensitivity (>99%), and very high specificity (>98%) for breast cancer. The accuracy, sensitivity, and specificity for status of healthy individuals, of individuals with prostate cancer, and of individuals with benign breast diseases were also very high.

TABLE 20

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9940 | 0.9961 | 0.9982 | 0.9848 | 0.9931 | 0.9904 | 264 |
| Cancer | | | | | | | |
| Vol (Healthy) | 0.9858 | 0.9843 | 0.9677 | 0.9947 | 0.9890 | 0.9895 | 569 |
| Prostate | 0.9916 | 0.75 | 0.9975 | 0.5454 | 0.9940 | 0.6315 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9885 | 0.9850 | 0.9777 | 0.9858 | 0.9904 | 0.9852 | 848 |

### Example 6: Test Dataset Containing 500 miRNA Measurements

Based on the results from Example 5, a set of the 500 miRNA with the highest prediction power was selected from the 600 miRNA biomarker set. A dataset containing measurements for the 500 miRNAs (all of the miRNAs listed in Tables 1-10) for each subject was used to train and evaluate a classifier using these 500 miRNA biomarkers as features. Using the results from the test set of 848 subjects, a confusion matrix was generated (FIG. 11) and performance metrics were calculated (shown in Table 21 below).

The results showed that the classifier developed using these 500 miRNA biomarkers had very high accuracy (>98%), very high sensitivity (>97%), and very high specificity (>99%) for breast cancer. The accuracy, sensitivity, and specificity for status of healthy individuals and of individuals with benign breast diseases were also very high.

TABLE 21

| | | | | | | | Total |
|---|---|---|---|---|---|---|---|
| | | Precision | Recall | | | | No. of |
| Class/metrics | Accuracy | (PPV) | (Sensitivity) | Specificity | NPV | F1-Score | subjects |
| Breast | 0.9893 | 0.9922 | 0.9734 | 0.9965 | 0.9880 | 0.9827 | 264 |
| Cancer | | | | | | | |
| Vol (Healthy) | 0.9834 | 0.9809 | 0.9947 | 0.9605 | 0.988 | 0.9877 | 569 |
| Prostate | 0.9940 | 0.875 | 0.6363 | 0.9987 | 0.9951 | 0.7368 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9855 | 0.9831 | 0.9834 | 0.9724 | 0.9887 | 0.9830 | 848 |

### Example 7: Test Dataset Containing 400 miRNA Measurements

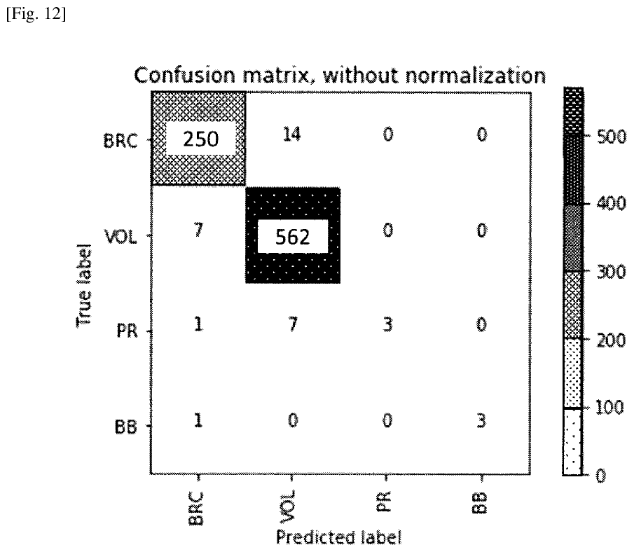

Based on the results from Example 6, a set of the 400 miRNA with the highest prediction power was selected from the 500 miRNA biomarker set. A dataset containing measurements for the 400 miRNAs (all of the miRNAs listed in Tables 1-9) for each subject was used to train and evaluate a classifier using these 400 miRNA biomarkers as features. Using the results from the test set of 848 subjects, a confusion matrix was generated (FIG. 12) and performance metrics were calculated (shown in Table 22 below).

The results showed that the classifier developed using these 500 miRNA biomarkers had very high accuracy (>97%), high sensitivity (>94%), and very high specificity (>98%) for breast cancer. The accuracy, sensitivity, and specificity for status of healthy individuals and of individuals with benign breast diseases were also sufficiently high.

TABLE 22

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9726 | 0.9652 | 0.9469 | 0.9844 | 0.9759 | 0.9560 | 264 |
| Cancer | | | | | | | |
| Vol (Healthy) | 0.9669 | 0.9639 | 0.9876 | 0.9241 | 0.9733 | 0.9756 | 569 |
| Prostate | 0.9903 | 1 | 0.2727 | 1 | 0.9902 | 0.4285 | 11 |
| Cancer | | | | | | | |
| Benign | 0.9987 | 1 | 0.75 | 1 | 0.9987 | 0.8571 | 4 |
| Breast | | | | | | | |
| Disease | | | | | | | |
| Avg/Total | 0.9691 | 0.9650 | 0.9646 | 0.9442 | 0.9745 | 0.9619 | 848 |

### Example 8: Test Dataset Containing 300 miRNA Measurements

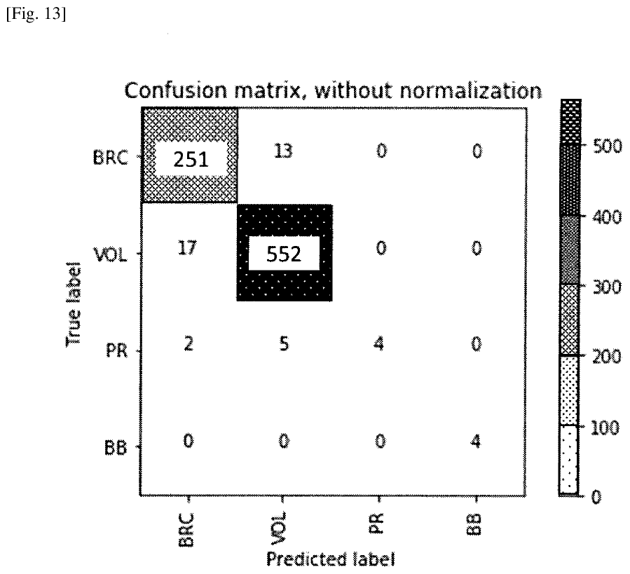

Based on the results from Example 7, a set of 300 miRNAs with the highest prediction power was selected from the 400 miRNA biomarker set. A dataset containing measurements for the 300 miRNAs (all of the miRNAs listed in Tables 1-8) for each subject was used to train and evaluate a classifier using these 300 miRNA biomarkers as features. Using the results from the test set of 848 subjects, a confusion matrix was generated (FIG. 13) and performance metrics were calculated (shown in Table 23 below).

The results showed that the classifier developed using these 300 miRNA biomarkers had very high accuracy (>96%), very high sensitivity (>95%), and very high specificity (>96%) for breast cancer. The accuracy, sensitivity, and specificity for status of healthy individuals and of individuals with benign breast diseases were also very high.

TABLE 23

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9620 | 0.9296 | 0.9507 | 0.9671 | 0.9773 | 0.9400 | 264 |
| Cancer | | | | | | | |
| Vol (Healthy) | 0.9586 | 0.9684 | 0.9701 | 0.9350 | 0.9384 | 0.9692 | 569 |
| Prostate | 0.9914 | 1 | 0.363636 | 1 | 0.9914 | 0.533333 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9603 | 0.9569 | 0.9563 | 0.9461 | 0.9514 | 0.9546 | 848 |

### Example 9: Test Dataset Containing 250 miRNA Measurements

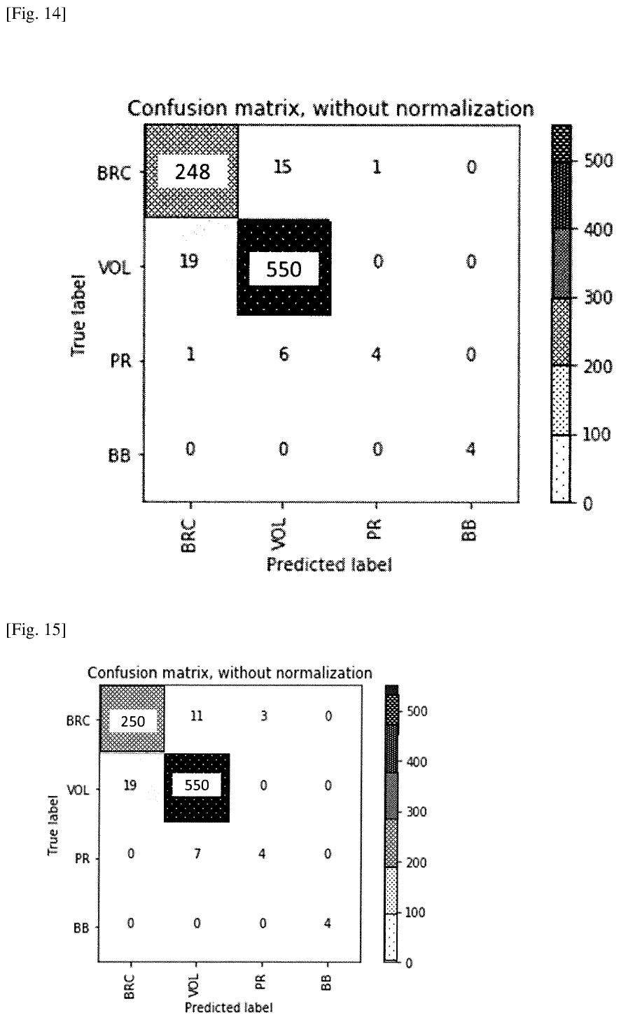

Based on the results from Example 8, a set of 250 miRNAs with the highest prediction power was selected from the 300 miRNA biomarker set. A dataset containing measurements for the 250 miRNAs (all of the miRNAs listed in Tables 1-7) for each subject was used to train and evaluate a classifier using these 250 miRNA biomarkers as features. Using the results from the test set of 848 subjects, a confusion matrix was generated (FIG. 14) and performance metrics were calculated (shown in Table 24 below).

The results showed that the classifier developed using these 250 miRNA biomarkers had very high accuracy (>95%), high sensitivity (>93%), and very high specificity (>96%) for breast cancer. The accuracy, sensitivity, and specificity for status of healthy individuals and of individuals with benign breast diseases were also very high.

TABLE 24

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9572 | 0.9253 | 0.9393 | 0.9653 | 0.9721 | 0.9323 | 264 |
| Cancer | | | | | | | |
| Vol (Healthy) | 0.9527 | 0.9632 | 0.9666 | 0.9241 | 0.9309 | 0.9649 | 569 |
| Prostate | 0.9901 | 0.8 | 0.3636 | 0.9987 | 0.9913 | 0.5 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9548 | 0.9494 | 0.9504 | 0.9383 | 0.9448 | 0.9489 | 848 |

### Example 10: Test Dataset Containing 200 miRNA Measurements

Based on the results from Example 9, a set of 200 miRNAs with the highest prediction power was selected from the 250 miRNA biomarker set. A dataset containing measurements for the 200 miRNAs (all of the miRNAs listed in Tables 1-6) for each subject was used to train and evaluate a classifier using these 200 miRNA biomarkers as features. Using the results from the test set of 848 subjects, a confusion matrix was generated (FIG. 15) and performance metrics were calculated (shown in Table 25 below).

The results showed that the classifier developed using these 200 miRNA biomarkers had very high accuracy (>96%), high sensitivity (>94%), and very high specificity (>96%) for breast cancer. The accuracy, sensitivity, and specificity for status of healthy individuals and of individuals with benign breast diseases were also very high.

TABLE 25

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9607 | 0.9293 | 0.9469 | 0.9670 | 0.9755 | 0.9380 | 264 |
| Cancer | | | | | | | |
| Vol (Healthy) | 0.9562 | 0.9683 | 0.9666 | 0.9347 | 0.9314 | 0.9674 | 569 |
| Prostate | 0.9877 | 0.5714 | 0.3636 | 0.9962 | 0.9913 | 0.4444 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9582 | 0.9511 | 0.9528 | 0.9459 | 0.9462 | 0.9516 | 848 |

### Example 11: Test Dataset Containing 150 miRNA Measurements

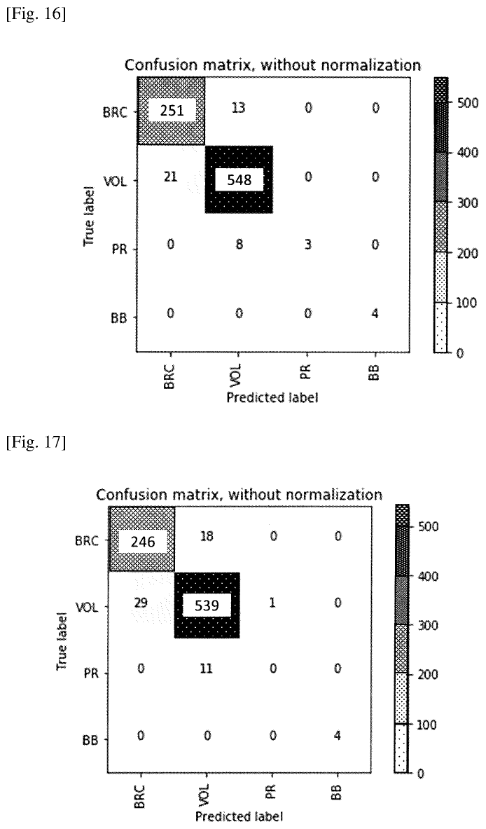

Based on the results from Example 10, as set of 150 miRNAs with the highest prediction power was selected from the 200 miRNA biomarker set. A dataset containing measurements for the 150 miRNAs (all of the miRNAs listed in Tables 1-5) for each subject was used to train and evaluate a classifier using these 150 miRNA biomarkers as features. Using the results from the test set of 848 subjects, a confusion matrix was generated (FIG. 16) and performance metrics were calculated (shown in Table 26 below).

The results showed that the classifier developed using these 150 miRNA biomarkers had very high accuracy (>95%), very high sensitivity (>95%), and very high specificity (>96%) for breast cancer. The accuracy, sensitivity, and specificity for status of healthy individuals and of individuals with benign breast diseases were also very high.

TABLE 26

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9595 | 0.9227 | 0.9507 | 0.9635 | 0.9771 | 0.9365 | 264 |
| Cancer | | | | | | | |
| Vol (Healthy) | 0.9504 | 0.9630 | 0.9630 | 0.9247 | 0.9247 | 0.9630 | 569 |
| Prostate | 0.9901 | 1 | 0.2727 | 1 | 0.9901 | 0.4285 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9540 | 0.9512 | 0.9504 | 0.9381 | 0.9422 | 0.9480 | 848 |

### Example 12: Test Dataset Containing 100 miRNA Measurements

Based on the results from Example 11, a set of 100 miRNAs with the highest prediction power was selected from the 150 miRNA biomarker set. A dataset containing measurements for the 100 miRNAs (all of the miRNAs listed in Tables 1-4) for each subject was used to train and evaluate a classifier using these 100 miRNA biomarkers as features. Using the results from the test set of 848 subjects, a confusion matrix was generated (FIG. 17) and performance metrics were calculated (shown in Table 27 below).

The results showed that the classifier using these 100 miRNA biomarkers had high accuracy (>94%), high sensitivity (>93%), and high specificity (>94%) for breast cancer. The accuracy, sensitivity, and specificity for status of healthy individuals and of individuals with benign breast diseases were also quite high.

TABLE 27

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9437 | 0.8945 | 0.9318 | 0.9493 | 0.9679 | 0.9128 | 264 |
| Cancer | | | | | | | |
| Vol (Healthy) | 0.9304 | 0.9489 | 0.9472 | 0.8960 | 0.8928 | 0.9481 | 569 |
| Prostate | 0.9850 | 0 | 0 | 0.9987 | 0.9862 | 0 | 11 |
| Cancer | | | | | | | |
| Benign | 1 | 1 | 1 | 1 | 1 | 1 | 4 |
| Breast | | | | | | | |
| Disease | | | | | | | |
| Avg/Total | 0.9356 | 0.9199 | 0.9304 | 0.9144 | 0.9179 | 0.9250 | 848 |

### Example 13: Test Dataset Containing 50 miRNA Measurements

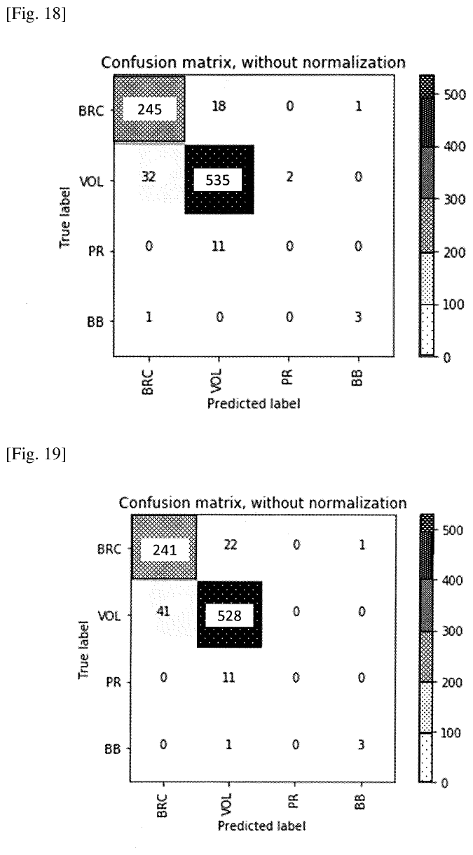

Based on the results from Example 12, a set of 50 miRNAs with the highest prediction power was selected from the 100 miRNA biomarker set. A dataset containing measurements for the 50 miRNAs (all of the miRNAs listed in Tables 1-3) for each subject was used to train and evaluate a classifier using these 50 miRNA biomarkers as features. Using the results from the test set of 848 subjects, a confusion matrix was generated (FIG. 18) and performance metrics were calculated (shown in Table 28 below).

The results showed that the classifier using these 50 miRNA biomarkers had high accuracy (>93%), high sensitivity (>92%), and high specificity (>94%) for breast cancer. The result shows that even a classifier with a small set of features can still have outstanding performance. The accuracy, sensitivity, and specificity for status of healthy individuals and of individuals with benign breast diseases were also sufficiently high.

TABLE 28

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9377 | 0.8812 | 0.9280 | 0.9422 | 0.9658 | 0.9040 | 264 |
| Cancer | | | | | | | |
| Vol (Healthy) | 0.9255 | 0.9485 | 0.9402 | 0.8953 | 0.8794 | 0.9443 | 569 |
| Prostate | 0.9836 | 0 | 0 | 0.9974 | 0.9861 | 0 | 11 |
| Cancer | | | | | | | |
| Benign | 0.9974 | 0.75 | 0.75 | 0.9987 | 0.998 | 0.75 | 4 |
| Breast | | | | | | | |
| Disease | | | | | | | |
| Avg/Total | 0.9304 | 0.9143 | 0.9233 | 0.9117 | 0.9082 | 0.9186 | 848 |

### Example 14: Test Dataset Containing 25 miRNA Measurements

Based on the results from Example 13, a set of 25 miRNAs with the highest prediction power was selected from the 50 miRNA biomarker set. A dataset containing measurements for the 25 miRNAs (all of the miRNAs listed in Tables 1-2) for each subject was used to train and evaluate a classifier using these 25 miRNA biomarkers as features. Using the results from the test set of 848 subjects, a confusion matrix was generated (FIG. 19) and performance metrics were calculated (shown in Table 29 below).

The results showed that the classifier using these 25 miRNA biomarkers had high accuracy (>92%), high sensitivity (>91%), and high specificity (>92%) for breast cancer. The result shows that even a classifier with a small set of features can still have outstanding performance. The accuracy, sensitivity, and specificity for status of healthy individuals and of individuals with benign breast diseases were also sufficiently high.

TABLE 29

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9234 | 0.8546 | 0.9128 | 0.9283 | 0.9584 | 0.8827 | 264 |
| Cancer | | | | | | | |
| Vol (Healthy) | 0.9114 | 0.9395 | 0.9279 | 0.8776 | 0.8561 | 0.9336 | 569 |
| Prostate | 0.9859 | 0 | 0 | 1 | 0.9859 | 0 | 11 |
| Cancer | | | | | | | |
| Benign | 0.9974 | 0.75 | 0.75 | 0.9987 | 0.998 | 0.75 | 4 |
| Breast | | | | | | | |
| Disease | | | | | | | |
| Avg/Total | 0.9165 | 0.75 | 0.9103 | 0.8956 | 0.8903 | 0.9048 | 848 |

### Example 15: Test Dataset Containing 10 miRNA Measurements

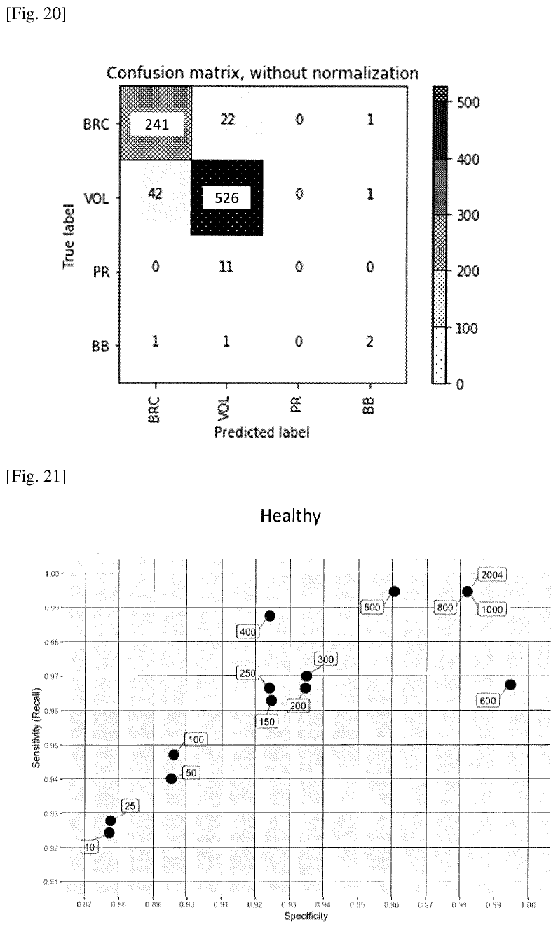

Based on the results from Example 14, a set of 10 miRNAs with the highest prediction power was selected from the 25 miRNA biomarker set. A dataset containing measurements for the 10 miRNAs (all of the miRNAs listed in Table 1) for each subject was used to train and evaluate a classifier using these 10 miRNA biomarkers as features. Using the results from the test set of 848 subjects, a confusion matrix was generated (FIG. 20) and performance metrics were calculated (shown in Table 30 below).

The results showed that the classifier using these 10 miRNA biomarkers had high accuracy (>92%), high sensitivity (>91%), and high specificity (>92%) for breast cancer. The result shows that even a classifier with a small set of features can still have outstanding performance. The accuracy, sensitivity, and specificity for status of healthy individuals and of individuals with benign breast diseases were also sufficiently high.

TABLE 30

| | | | | | | | Total |
|---|---|---|---|---|---|---|---|
| | | Precision | Recall | | | | No. of |
| Class/Metrics | Accuracy | (PPV) | (Sensitivity) | Specificity | NPV | F1-Score | subjects |
| Breast | 0.9209 | 0.8485 | 0.9128 | 0.9246 | 0.9582 | 0.8795 | 264 |
| Cancer | | | | | | | |
| Vol (Healthy) | 0.9089 | 0.9392 | 0.9244 | 0.8772 | 0.8496 | 0.9317 | 569 |
| Prostate | 0.9858 | 0.0 | 0.0 | 1 | 0.9858 | 0.0 | 11 |
| Cancer | | | | | | | |
| Benign | 0.9948 | 0.5 | 0.5 | 0.9973 | 0.9973 | 0.5 | 4 |
| Breast | | | | | | | |
| Disease | | | | | | | |
| Avg/Total | 0.9141 | 0.9 | 0.9068 | 0.8941 | 0.8859 | 0.9014 | 848 |

### Example 16: Sensitivity and Specificity of Different Classifiers

The sensitivity and specificity of different classifiers in predicting healthy individuals, individuals having breast cancer, and individuals having a benign breast disease were analyzed and compared. The biomarker sets used in Examples 2-15 are summarized in the table below.

TABLE 31

| | Number of | The tables in which the miRNAs |
|---|---|---|
| Examples | miRNAs | are listed |
| Example 2 | 2004 miRNA set | Tables 1-14 |
| | | (Table 14 includes additional biomarkers |
| | | as compared to the 1000 miRNA set) |
| Example 3 | 1000 miRNA set | Tables 1-13 |
| | | (Table 13 includes additional biomarkers |
| | | as compared to the 800 miRNA set) |
| Example 4 | 800 miRNA set | Tables 1-12 |
| | | (Table 12 includes additional biomarkers |
| | | as compared to the 600 miRNA set) |
| Example 5 | 600 miRNA set | Tables 1-11 |
| | | (Table 11 includes additional biomarkers |
| | | as compared to the 500 miRNA set) |
| Example 6 | 500 miRNA set | Tables 1-10 |
| | | (Table 10 includes additional biomarkers |
| | | as compared to the 400 miRNA set) |
| Example 7 | 400 miRNA set | Tables 1-9 |
| | | (Table 9 includes additional biomarkers |
| | | as compared to the 300 miRNA set) |
| Example 8 | 300 miRNA set | Tables 1-8 |
| | | (Table 8 includes additional biomarkers |
| | | as compared to the 250 miRNA set) |
| Example 9 | 250 miRNA set | Tables 1-7 |
| | | (Table 7 includes additional biomarkers |
| | | as compared to the 200 miRNA set) |
| Example 10 | 200 miRNA set | Tables 1-6 |
| | | (Table 6 includes additional biomarkers |
| | | as compared to the 150 miRNA set) |
| Example 11 | 150 miRNA set | Tables 1-5 |
| | | (Table 5 includes additional biomarkers |
| | | as compared to the 100 miRNA set) |
| Example 12 | 100 miRNA set | Tables 1-4 |
| | | (Table 4 includes additional biomarkers |
| | | as compared to the 50 miRNA set) |
| Example 13 | 50 miRNA set | Tables 1-3 |
| | | (Table 3 includes additional biomarkers |
| | | as compared to the 25 miRNA set) |
| Example 14 | 25 miRNA set | Tables 1-2 |
| | | (Table 2 includes additional biomarkers |
| | | as compared to the 10 miRNA set) |
| Example 15 | 10 miRNA set | Table 1 |

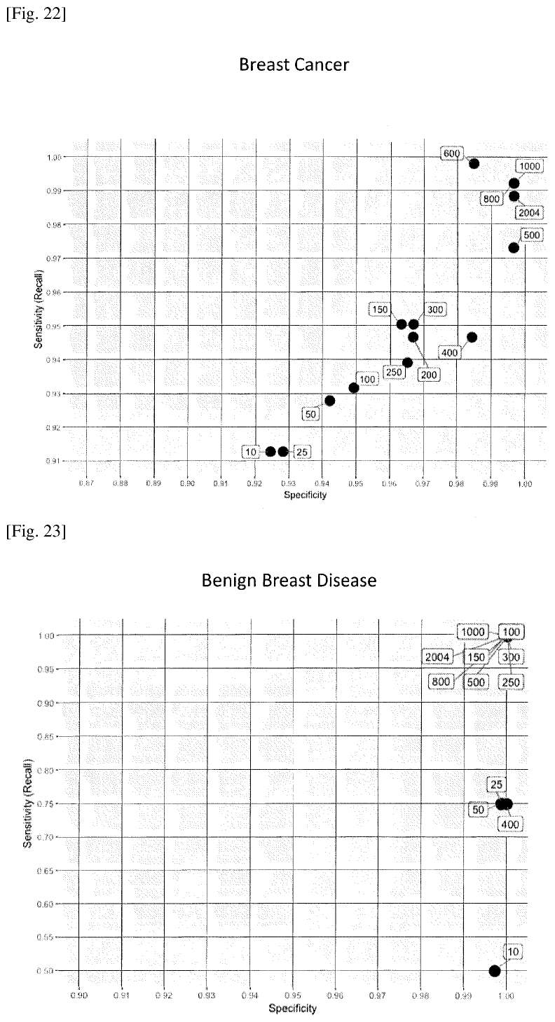

The results are compared in FIGS. 21-23. Each dot in those figures is labeled with a number representing the number of miRNAs in the set. These results show that increasing the number of miRNA biomarkers is generally correlated with increased specificity and sensitivity for predicting which individuals have breast cancer or benign breast disease or neither. Surprisingly, classifiers with a much smaller number of biomarkers (e.g., 10-50) still have sufficiently high specificity and sensitivity for predicting which individuals have breast cancer.

### OTHER EMBODIMENTS

It is to be understood that, while the disclosure has been described in conjunction with the detailed description thereof, the foregoing description is intended to illustrate and not limit the scope of the disclosure. For example, implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible program carrier for execution by, or to control the operation of, a processing device. Alternatively, or in addition, the program instructions can be encoded on a propagated signal that is an artificially generated signal, e.g., a machine-generated electrical, optical, or electromagnetic signal that is generated to encode information for transmission to suitable receiver apparatus for execution by a processing device. A machine-readable medium can be a machine-readable storage device, a machine-readable storage substrate, a random or serial access memory device, or a combination of one or more of them.

In some embodiments, various methods and formulae are implemented, in the form of computer program instructions, and executed by a processing device. Suitable programming languages for expressing the program instructions include, but are not limited to, C, C++, an embodiment of FORTRAN such as FORTRAN77 or FORTRAN90, Java, Visual Basic, Perl, Tcl/Tk, JavaScript, ADA, and statistical analysis software, such as SAS, R, MATLAB, SPSS, and Stata etc. Various aspects of the methods may be written in different computing languages from one another, and the various aspects are caused to communicate with one another by appropriate system-level-tools available on a given system.

The processes and logic flows described in this specification can be performed by one or more programmable computers executing one or more computer programs to perform functions by operating on input information and generating output. The processes and logic flows can also be performed by, and apparatus can also be implemented as, special purpose logic circuitry, e.g., an FPGA (field programmable gate array) or an ASIC (application specific integrated circuit) or RISC.

Computers suitable for the execution of a computer program include, by way of example, general or special purpose microprocessors, or both, or any other kind of central processing unit. Generally, a central processing unit will receive instructions and information from a read only memory or a random access memory or both. The essential elements of a computer are a central processing unit for performing or executing instructions and one or more memory devices for storing instructions and information. Generally, a computer will also include, or be operatively coupled to receive information from or transfer information to, or both, one or more mass storage devices for storing information, e.g., magnetic, magneto optical disks, or optical disks. However, a computer need not have such devices. Moreover, a computer can be embedded in another device, e.g., a mobile telephone, a smartphone or a tablet, a touchscreen device or surface, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and information include various forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and (Blue Ray) DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back end component, e.g., as an information server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital information communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, the server can be in the cloud via cloud computing services.

While this specification includes many specific implementation details, these should not be construed as limitations on the scope of any of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. In one embodiment, the processes depicted in the accompanying figures do not necessarily require the particular order shown, or sequential order, to achieve desirable results. In some implementations, multitasking and parallel processing may be advantageous. Accordingly, other aspects, advantages, and modifications are within the scope of the following claims.

This application claims priority to U.S. Provisional Application No. 62/871,604, entitled “DATA PROCESSING AND CLASSIFICATION FOR DETERMINING A LIKELIHOOD SCORE FOR BREAST DISEASE”, filed on Jul. 8, 2019, the contents of which are hereby incorporated by reference herein in its entirety.

1. A computer-implemented method for processing data in one or more data processing devices to determine a likelihood score that a subject has breast cancer, the method comprising:
inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals, each of whom has breast cancer, or with (B) expression levels of the same set of non-coding RNAs in biological samples collected from a second group of individuals, none of whom has breast cancer; and
determining, by the one or more data processing devices, based on application of the classifier, the likelihood score that the subject has breast cancer,
wherein the set of non-coding RNAs comprises five or more non-coding RNAs selected from Table 1.
TABLE 1
hsa-miR-495-3p
hsa-miR-1976
hsa-miR-1973
hsa-miR-1972
hsa-miR-3065-3p
hsa-miR-4778-3p
hsa-miR-1914-3p
hsa-miR-3689a-5p, hsa-miR-3689b-5p, or hsa-miR-3689e
hsa-miR-4999-3p
hsa-miR-6873-3p

2. The computer-implemented method of claim 1, wherein the set of non-coding RNAs comprises 10 non-coding RNAs from Table 1.

3. The computer-implemented method of claim 1, wherein the set of non-coding RNAs further comprises one or more non-coding RNAs from Table 2.
TABLE 2
hsa-miR-6841-5p
hsa-miR-552-5p
hsa-miR-6817-5p
hsa-miR-4633-5p
hsa-miR-4704-3p
hsa-miR-671-3p
hsa-miR-514a-3p
hsa-miR-6864-5p
hsa-miR-4529-3p
hsa-miR-216b-3p
hsa-miR-509-3p
hsa-miR-526b-3p
hsa-miR-29b-1-5p
hsa-miR-5587-5p
hsa-miR-216a-3p

4. The computer-implemented method of claim 3, wherein the set of non-coding RNAs further comprises one or more non-coding RNAs from Table 3.
TABLE 3

| | |
|---|---|
| hsa-miR-33b-3p | hsa-miR-4494 |
| hsa-miR-4773 | hsa-miR-4493 |
| hsa-miR-302c-3p | hsa-miR-4491 |
| hsa-miR-4477b | hsa-miR-4490 |
| hsa-miR-4477a | hsa-miR-514b-5p |
| hsa-miR-96-3p | hsa-miR-1251-3p |
| hsa-miR-4715-3p | hsa-miR-4499 |
| hsa-miR-5004-3p | hsa-miR-4498 |
| hsa-miR-4482-5p | hsa-miR-4654 |
| hsa-miR-1273g-5p | hsa-miR-4657 |
| hsa-miR-151b | hsa-miR-5708 |
| hsa-miR-4496 | hsa-miR-6795-3p |
| hsa-miR-4495 | |

5. The computer-implemented method of claim 4, wherein the set of non-coding RNAs further comprises one or more non-coding RNAs from Table 4.
TABLE 4

| | | | |
|---|---|---|---|
| hsa-miR-205-3p | hsa-miR-6789-3p | hsa-miR-6764-5p | hsa-miR-519e-5p |
| hsa-miR-5704 | hsa-miR-6773-5p | hsa-miR-433-3p | hsa-miR-6831-5p |
| hsa-miR-5705 | hsa-miR-4796-3p | hsa-miR-455-5p | hsa-miR-4746-5p |
| hsa-miR-5706 | hsa-miR-3678-3p | hsa-miR-6882-3p | hsa-miR-6739-5p |
| hsa-miR-5707 | hsa-miR-630 | hsa-miR-3141 | hsa-miR-6886-3p |
| hsa-miR-5700 | hsa-miR-631 | hsa-miR-3142 | |
| hsa-miR-4658 | hsa-miR-632 | hsa-miR-2682-5p | |
| hsa-miR-5702 | hsa-miR-633 | hsa-miR-7853-5p | |
| hsa-miR-5703 | hsa-miR-635 | hsa-miR-3136-3p | |
| hsa-miR-4708-3p | hsa-miR-6508-3p | hsa-miR-3147 | |
| hsa-miR-6815-5p | hsa-miR-637 | hsa-miR-3148 | |
| hsa-miR-4290 | hsa-miR-639 | hsa-miR-3149 | |
| hsa-miR-4527 | hsa-miR-6762-3p | hsa-miR-208b-5p | |
| hsa-miR-8077 | hsa-miR-5588-5p | hsa-miR-6878-5p | |
| hsa-miR-8076 | hsa-miR-139-3p | hsa-miR-3617-5p | |

6. The computer-implemented method of claim 5, wherein the set of non-coding RNAs further comprises one or more non-coding RNAs from Table 5.
TABLE 5

| | | | |
|---|---|---|---|
| hsa-miR-3925-3p | hsa-miR-3921 | hsa-miR-4655-3p | hsa-miR-6826-3p |
| hsa-miR-708-5p | hsa-miR-3926 | hsa-miR-1285-5p | hsa-miR-4786-5p |
| hsa-miR-2467-3p | hsa-miR-3924 | hsa-miR-3145-5p | hsa-miR-4753-3p |
| hsa-miR-214-5p | hsa-miR-4251 | hsa-miR-125a-5p | hsa-miR-548ar-3p |
| hsa-miR-215-5p | hsa-miR-6761-3p | hsa-miR-3144-5p | hsa-miR-7-l-3p |
| hsa-miR-548aa, or | hsa-miR-4253 | hsa-miR-4700-3p | |
| hsa-miR-548t-3p | | | |
| hsa-miR-6822-3p | hsa-miR-3929 | hsa-miR-7856-5p | |
| hsa-miR-3124-3p | hsa-miR-3156-3p | hsa-miR-1178-3p | |
| hsa-miR-3158-5p | hsa-miR-4254 | hsa-miR-593-3p | |
| hsa-miR-515-5p | hsa-miR-4256 | hsa-miR-204-5p | |
| hsa-miR-6821-3p | hsa-miR-16-l-3p | hsa-miR-6866-5p | |
| hsa-miR-6834-3p | hsa-miR-4735-5p | hsa-miR-7-2-3p | |
| hsa-miR-627-3p | hsa-miR-4757-3p | hsa-miR-6867-3p | |
| hsa-miR-3923 | hsa-miR-3187-5p | hsa-miR-544a | |
| hsa-miR-3920 | hsa-miR-539-3p | hsa-miR-338-3p | |

7. The computer-implemented method of claim 6, wherein the set of non-coding RNAs further comprises one or more non-coding RNAs from Table 6.
TABLE 6

| | | | |
|---|---|---|---|
| hsa-miR-133a-5p | hsa-miR-619-3p | hsa-miR-6843-3p | hsa-miR-6086 |
| hsa-miR-3200-5p | hsa-miR-656-3p | hsa-miR-7151-5p | hsa-miR-6084 |
| hsa-miR-2277-3p | hsa-miR-3591-3p | hsa-miR-1292-3p | hsa-miR-6082 |
| hsa-miR-6509-5p | hsa-miR-6833-5p | hsa-miR-548ao-5p | hsa-miR-6083 |
| hsa-miR-7109-3p | hsa-miR-4710 | hsa-miR-642b-3p | hsa-miR-6080 |
| hsa-miR-372-3p | hsa-miR-3913-5p | hsa-miR-6516-5p | |
| hsa-miR-365b-5p | hsa-miR-6744-3p | hsa-miR-376c-3p | |
| hsa-miR-6839-5p | hsa-miR-6737-3p | hsa-miR-7113-3p | |
| hsa-miR-6860 | hsa-miR-4718 | hsa-miR-487a-3p | |
| hsa-miR-193a-3p | hsa-miR-4719 | hsa-miR-3912-3p | |
| hsa-miR-6838-3p | hsa-miR-3151-5p | hsa-miR-5008-5p | |
| hsa-miR-6863 | hsa-miR-34c-3p | hsa-miR-130a-5p | |
| hsa-miR-541-5p | hsa-miR-1306-5p | hsa-miR-520c-3p | |
| hsa-miR-517a-3p, or | hsa-miR-4652-3p | hsa-miR-7843-5p | |
| hsa-miR-517b-3p | | | |
| hsa-miR-6791-3p | hsa-miR-4262 | hsa-miR-5003-5p | |

8. The computer-implemented method of claim 7, wherein the set of non-coding RNAs further comprises one or more non-coding RNAs from Table 7.
TABLE 7

| | | | |
|---|---|---|---|
| hsa-miR-6081 | hsa-miR-466 | hsa-miR-381-3p | hsa-miR-3189-3p |
| hsa-miR-6847-3p | hsa-miR-3681-3p | hsa-miR-892c-3p | hsa-miR-3939 |
| hsa-miR-5006-3p | hsa-miR-624-3p | hsa-miR-5189-3p | hsa-miR-3938 |
| hsa-miR-6743-3p | hsa-miR-7153-5p | hsa-miR-942-3p | hsa-miR-3935 |
| hsa-miR-607 | hsa-miR-561-5p | hsa-miR-6512-3p | hsa-miR-219a-2-3p |
| hsa-miR-1296-3p | hsa-miR-6847-5p | hsa-miR-6813-3p | |
| hsa-miR-4538 | hsa-miR-3153 | hsa-miR-6769b-3p | |
| hsa-miR-4539 | hsa-miR-513c-3p | hsa-miR-3126-5p | |
| hsa-miR-603 | hsa-miR-6501-5p | hsa-miR-374c-5p | |
| hsa-miR-4535 | hsa-miR-4646-5p | hsa-miR-4665-3p | |
| hsa-miR-4537 | hsa-miR-3159 | hsa-miR-499a-3p | |
| hsa-miR-609 | hsa-miR-6747-3p | hsa-miR-4712-5p | |
| hsa-miR-4531 | hsa-miR-208a-3p | hsa-miR-4697-5p | |
| hsa-miR-4533 | hsa-miR-548e-5p | hsa-miR-4777-5p | |
| hsa-miR-5000-5p | hsa-miR-548d-3p | hsa-miR-146a-3p | |

9. The computer-implemented method of claim 8, wherein the set of non-coding RNAs further comprises one or more non-coding RNAs from Table 8.
TABLE 8

| | | | |
|---|---|---|---|
| hsa-miR-3936 | hsa-miR-181d-5p | hsa-miR-3689d | hsa-miR-549a |
| hsa-miR-4684-5p | hsa-miR-888-5p | hsa-miR-545-3p | hsa-miR-5584-3p |
| hsa-miR-4436b-3p | hsa-miR-4800-5p | hsa-miR-4799-5p | hsa-miR-4704-5p |
| hsa-miR-4685-3p | hsa-miR-548f-5p | hsa-miR-3065-5p | hsa-miR-188-3p |
| hsa-miR-548aj-3p | hsa-miR-124-3p | hsa-miR-105-5p | hsa-miR-4529-5p |
| hsa-miR-431-3p | hsa-miR-6890-3p | hsa-miR-4798-5p | |
| hsa-miR-4661-3p | hsa-miR-4694-5p | hsa-miR-4778-5p | |
| hsa-miR-1179 | hsa-miR-181a-3p | hsa-miR-4707-5p | |
| hsa-miR-4790-3p | hsa-miR-5701 | hsa-miR-4433b-3p | |
| hsa-miR-6893-3p | hsa-miR-6830-5p | hsa-miR-6845-5p | |
| hsa-miR-200a-5p | hsa-miR-550b-3p | hsa-miR-548ap-3p | |
| hsa-miR-1273h-3p | hsa-miR-3171 | hsa-miR-3714 | |
| hsa-miR-548av-3p | hsa-miR-5571-3p | hsa-miR-302b-5p | |
| hsa-miR-766-3p | hsa-miR-3689f | hsa-miR-3713 | |
| hsa-miR-6872-5p | hsa-miR-495-5p | hsa-miR-664b-3p | |

10. The computer-implemented method of claim 9, wherein the set of non-coding RNAs further comprises one or more non-coding RNAs from Table 9.
TABLE 9

| | | | |
|---|---|---|---|
| hsa-miR-6749-3p | hsa-miR-298 | hsa-miR-3675-5p | hsa-miR-6504-3p |
| hsa-miR-664a-3p | hsa-miR-297 | hsa-miR-216a-5p | hsa-miR-1271-5p |
| hsa-miR-6865-5p | hsa-miR-4640-5p | hsa-miR-31-3p | hsa-miR-7157-3p |
| hsa-miR-4705 | hsa-miR-1468-3p | hsa-miR-6828-3p | hsa-miR-2467-5p |
| hsa-miR-3120-3p | hsa-miR-3152-5p | hsa-miR-4722-5p | hsa-miR-215-3p |
| hsa-miR-518a-3p | hsa-miR-488-3p | hsa-miR-3168 | hsa-miR-6785-3p |
| hsa-miR-5587-3p | hsa-miR-744-3p | hsa-miR-3169 | hsa-miR-4802-5p |
| hsa-miR-6811-3p | hsa-miR-612 | hsa-miR-3166 | hsa-miR-25-5p |
| hsa-miR-138-1-3p | hsa-miR-613 | hsa-miR-3167 | hsa-miR-218-2-3p |
| hsa-miR-2392 | hsa-miR-610 | hsa-miR-3164 | hsa-miR-1288-3p |
| hsa-miR-371b-3p | hsa-miR-611 | hsa-miR-3165 | hsa-miR-6727-3p |
| hsa-miR-5089-3p | hsa-miR-450b-5p | hsa-miR-3163 | hsa-miR-7855-5p |
| hsa-miR-519b-3p | hsa-miR-617 | hsa-miR-3161 | hsa-miR-7159-3p |
| hsa-miR-302a-5p | hsa-miR-6508-5p | hsa-miR-3136-5p | hsa-miR-4330 |
| hsa-miR-651-5p | hsa-miR-5580-3p | hsa-miR-6799-3p | hsa-miR-4768-5p |
| hsa-miR-6502-5p | hsa-miR-100-5p | hsa-miR-507 | hsa-miR-548au-5p |
| hsa-miR-514b-3p | hsa-miR-4528 | hsa-miR-1225-5p | hsa-miR-1228-3p |
| hsa-miR-654-3p | hsa-miR-510-3p | hsa-miR-605-3p | hsa-miR-3941 |
| hsa-miR-4637 | hsa-miR-4526 | hsa-miR-636 | hsa-miR-3943 |
| hsa-miR-4636 | hsa-miR-4523 | hsa-miR-1224-3p | hsa-miR-3945 |
| hsa-miR-4635 | hsa-miR-4522 | hsa-miR-6716-3p | hsa-miR-8053 |
| hsa-miR-367-3p | hsa-miR-4521 | hsa-miR-1277-3p | hsa-miR-8052 |
| hsa-miR-518e-3p | hsa-miR-520g-3p | hsa-miR-4711-5p | hsa-miR-6761-5p |
| hsa-miR-652-5p | hsa-miR-4709-3p | hsa-miR-3064-5p | hsa-miR-8057 |
| hsa-miR-6815-3p | hsa-miR-373-5p | hsa-miR-6739-3p | hsa-miR-8056 |

11. The computer-implemented method of claim 10, wherein the set of non-coding RNAs further comprises one or more non-coding RNAs from Table 10.
TABLE 10

| | | | |
|---|---|---|---|
| hsa-miR-8055 | hsa-miR-597-5p | hsa-miR-2115-3p | hsa-miR-4511 |
| hsa-miR-6781-3p | hsa-miR-133a-3p | hsa-miR-548n | hsa-miR-4516 |
| hsa-miR-8058 | hsa-miR-138-5p | hsa-miR-548m | hsa-miR-3150a-3p |
| hsa-miR-7161-3p | hsa-miR-6778-3p | hsa-miR-5481 | hsa-miR-4514 |
| hsa-miR-3677-5p | hsa-miR-1236-3p | hsa-miR-548k | hsa-miR-4515 |
| hsa-miR-4666a-5p | hsa-miR-1266-5p | hsa-miR-548i | hsa-miR-550a-5p |
| hsa-miR-6841-3p | hsa-miR-323a-3p | hsa-miR-642b-5p | hsa-miR-409-5p |
| hsa-miR-6856-3p | hsa-miR-4536-5p | hsa-miR-376c-5p | hsa-miR-5100 |
| hsamiR-6796-5p | hsa-miR-6838-5p | hsa-miR-548y | hsa-miR-4677-5p |
| hsa-miR-3144-3p | hsa-miR-6844 | hsa-miR-548w | hsa-miR-6503-5p |
| hsa-miR-513b-5p | hsa-miR-132-3p | hsa-miR-548v | hsa-miR-6755-3p |
| hsa-miR-4756-3p | hsa-miR-670-3p | hsa-miR-548u | hsa-miR-7153-3p |
| hsa-miR-6510-3p | hsa-miR-4436a | hsa-miR-5739 | hsa-miR-7160-5p |
| hsa-miR-6730-5p | hsa-miR-1304-5p | hsa-miR-548q | hsa-miR-6793-3p |
| hsa-miR-1260a | hsa-miR-4762-3p | hsa-miR-548p | hsa-miR-5002-5p |
| hsa-miR-6867-5p | hsa-miR-6827-5p | hsa-miR-9-5p | hsa-miR-3179 |
| hsa-miR-574-5p | hsa-miR-4782-3p | hsa-miR-598-5p | hsa-miR-128-3p |
| hsa-miR-181b-3p | hsa-miR-4737 | hsa-miR-4793-5p | hsa-miR-95-3p |
| hsa-miR-4755-3p | hsa-miR-2116-3p | hsa-miR-6068 | hsa-miR-451b |
| hsa-miR-4655-5p | hsa-miR-1301-3p | hsa-miR-6069 | hsa-miR-3170 |
| hsa-miR-4786-3p | hsa-miR-5195-5p | hsa-miR-662 | hsa-miR-642a-5p |
| hsa-miR-3140-3p | hsa-miR-1307-5p | hsa-miR-4518 | hsa-miR-451a |
| hsa-miR-4753-5p | hsa-miR-3146 | hsa-miR-4519 | hsa-miR-3175 |
| hsa-miR-146a-5p | hsa-miR-3689b-3p, | hsa-miR-4512 | hsa-miR-3174 |
| | or hsa-miR-3689c | | |
| hsa-miR-6869-3p | hsa-miR-4667-5p | hsa-miR-4510 | hsa-miR-3176 |

12. The computer-implemented method of claim 11, wherein the set of non-coding RNAs further comprises one or more non-coding RNAs from Table 11.
TABLE 11

| | | | |
|---|---|---|---|
| hsa-miR-4720-5p | hsamiR-331-3p | hsa-miR-4285 | hsa-miR-4472 |
| hsa-miR-4646-3p | hsa-miR-6852-3p | hsa-miR-4288 | hsa-miR-6804-5p |
| hsa-miR-514a-5p | hsa-miR-219a-5p | hsa-miR-4289 | hsa-miR-4798-3p |
| hsa-miR-6753-5p | hsa-miR-452-3p | hsa-miR-7852-3p | hsa-miR-615-3p |
| hsa-miR-6500-5p | hsa-miR-6133 | hsa-miR-127-5p | hsa-miR-3605-5p |
| hsa-miR-4781-3p | hsa-miR-3130-3p | hsa-miR-6718-5p | hsa-miR-1911-3p |
| hsa-miR-541-3p | hsa-miR-23b-5p | hsa-miR-4479 | hsa-miR-664b-5p |
| hsamiR 6735-3p | hsa-miR-4777-3p | hsa-miR-4478 | hsa-miR-6733-3p |
| hsa-miR-106a-5p | hsa-miR-4766-3p | hsa-miR-766-5p | hsa-miR-493-3p |
| hsa-miR-7155-3p | hsa-miR-4713-5p | hsa-miR-3922-3p | hsa-miR-548b-3p |
| hsa-miR-548d-5p | hsa-miR-524-3p | hsa-miR-6758-5p | hsa-miR-7706 |
| hsa-miR-381-5p | hsa-miR-3691-5p | hsa-miR-888-3p | hsa-miR-7705 |
| hsa-miR-3117-5p | hsa-miR-4632-3p | hsa-miR-6759-3p | hsa-miR-7702 |
| hsa-miR-5189-5p | hsa-miR-8067 | hsa-miR-3664-5p | hsa-miR-7703 |
| hsa-miR-942-5p | hsa-miR-548x-3p | hsa-miR-548f-3p | hsa-miR-6865-3p |
| hsa-miR-6848-3p | hsa-miR-8065 | hsa-miR-6733-5p | hsa-miR-5579-3p |
| hsa-miR-30c-l-3p | hsa-miR-520f-5p | hsa-miR-5589-5p | hsa-miR-4729 |
| hsa-miR-548j-3p | hsa-miR-8060 | hsa-miR-5591-5p | hsa-miR-1245a |
| hsa-miR-3126-3p | hsa-miR-4716-3p | hsa-miR-4695-3p | hsa-miR-20a-3p |
| hsa-miR-4691-3p | hsa-miR-383-5p | hsa-miR-4743-5p | hsa-miR-4721 |
| hsa-miR-103b | hsa-miR-4283 | hsa-miR-6748-5p | hsa-miR-380-5p |
| hsa-miR-342-3p | hsa-miR-4280 | hsa-miR-5571-5p | hsa-miR-6814-5p |
| hsa-miR-5187-3p | hsa-miR-4281 | hsa-miR-648 | hsa-miR-3649 |
| hsa-miR-4774-3p | hsa-miR-4287 | hsa-miR-6846-5p | hsa-miR-5010-3p |
| hsa-miR-1238-3p | hsa-miR-4284 | hsa-miR-6742-5p | hsa-miR-3648 |

13. The computer-implemented method of claim 12, wherein the set of non-coding RNAs further comprises one or more non-coding RNAs from Table 12.
TABLE 12-1
TABLE 12

| | | | |
|---|---|---|---|
| hsa-miR-660-3p | hsa-miR-2681-5p | hsa-miR-19b-2-5p | hsa-miR-645 |
| hsa-miR-6767-3p | hsa-miR-4763-5p | hsa-miR-5196-3p | hsa-miR-647 |
| hsa-miR-497-3p | hsa-miR-4639-3p | hsa-miR-376b-3p | hsa-miR-646 |
| hsa-miR-1207-3p | hsa-miR-5583-5p | hsa-miR-2909 | hsa-miR-649 |
| hsa-miR-6832-5p | hsa-miR-1271-3p | hsa-miR-4645-5p | hsa-miR-2114-5p |
| hsa-miR-6884-5p | hsa-miR-5582-5p | hsa-miR-6797-5p | hsa-miR-29a-5p |
| hsa-miR-6502-3p | hsa-miR-503-5p | hsa-miR-6763-3p | hsa-miR-5001-3p |
| hsa-miR-6885-3p | hsa-miR-512-5p | hsa-miR-6750-5p | hsa-miR-6855-5p |
| hsa-miR-3151-3p | hsa-miR-6862-3p | hsa-miR-597-3p | hsa-miR-19b-l-5p |
| hsa-miR-3607-5p | hsa-miR-345-3p | hsa-miR-4315 | hsa-miR-4436b-5p |
| hsa-miR-6861-3p | hsa-miR-10b-3p | hsa-miR-6715b-3p | hsa-miR-3127-5p |
| hsa-miR-6774-5p | hsa-miR-890 | hsa-miR-668-3p | hsa-miR-4423-5p |
| hsa-miR-519d-3p | hsa-miR-4650-3p | hsa-miR-3944-5p | hsa-miR-2682-3p |
| hsa-miR-367-5p | hsa-miR-7159-5p | hsa-miR-6731-5p | hsa-miR-5002-3p |
| hsa-miR-222-5p | hsa-miR-4768-3p | hsa-miR-323a-5p | hsa-miR-519c-3p |
| hsa-miR-6801-3p | hsa-miR-105-3p | hsa-miR-6798-3p | hsa-miR-98-5p |
| hsa-miR-3152-3p | hsa-miR-6505-3p | hsa-miR-520d-5p | hsa-miR-3193 |
| hsa-miR-6077 | hsa-miR-8079 | hsa-miR-670-5p | hsa-miR-4761-5p |
| hsa-miR-6076 | hsa-miR-8078 | hsa-miR-4762-5p | hsa-miR-644a |
| hsa-miR-6074 | hsa-miR-4299 | hsa-miR-4759 | hsa-miR-548az-3p |
| hsa-miR-6073 | hsa-miR-3194-3p | hsa-miR-4676-5p | hsa-miR-187-3p |
| hsa-miR-6072 | hsa-miR-4295 | hsa-miR-22-5p | hsa-miR-7114-3p |
| hsa-miR-6071 | hsa-miR-4294 | hsa-miR-4751 | hsa-miR-3199 |
| hsa-miR-6070 | hsa-miR-4297 | hsa-miR-4752 | hsa-miR-3198 |
| hsa-miR-6751-3p | hsa-miR-4296 | hsa-miR-4754 | hsa-miR-6753-3p |
| hsa-miR-6078 | hsa-miR-4291 | hsa-miR-3190-3p | hsa-miR-4670-3p |
| hsa-miR-6745 | hsa-miR-8074 | hsa-miR-4701-3p | hsa-miR-4781-5p |

TABLE 12-2

| | | | |
|---|---|---|---|
| hsa-miR-6807-5p | hsa-miR-4293 | hsa-miR-2116-5p | hsa-miR-4667-3p |
| hsa-miR-6894-5p | hsa-miR-4292 | hsa-miR-219a-1-3p | hsa-miR-132-5p |
| hsa-miR-5580-5p | hsa-miR-7161-5p | hsa-miR-4699-5p | hsa-miR-4731-3p |
| hsa-miR-516b-5p | hsa-miR-4736 | hsa-miR-1295a | hsa-miR-520a-3p |
| hsa-miR-4504 | hsa-miR-6780a-3p | hsa-miR-7108-3p | hsa-miR-675-3p |
| hsa-miR-510-5p | hsa-miR-6506-3p | hsa-miR-449b-5p | hsa-miR-526a, |
| | | | hsa-miR-520c-5p, |
| | | | or hsa-miR-518d-5p |
| hsa-miR-4506 | hsa-miR-4714-3p | hsa-miR-6824-3p | hsa-miR-196b-3p |
| hsa-miR-4474-3p | hsa-miR-6881-3p | hsa-miR-122-3p | hsa-miR-6130 |
| hsa-miR-4500 | hsa-miR-4761-3p | hsa-miR-449c-5p | hsa-miR-6850-3p |
| hsa-miR-4503 | hsa-miR-6856-5p | hsa-miR-876-3p | hsa-miR-219b-3p |
| hsa-miR-4502 | hsa-miR-155-3p | hsa-miR-1295b-3p | hsa-miR-6134 |
| hsa-miR-487b-3p | hsa-miR-520g-5p | hsa-miR-3975 | hsa-miR-3619-5p |
| hsa-miR-4509 | hsa-miR-1229-3p | hsa-miR-1227-3p | hsa-miR-6741-3p |
| hsa-miR-4709-5p | hsa-miR-6515-5p | hsa-miR-3186-3p | hsa-miR-1587 |
| hsa-miR-582-3p | hsa-miR-4756-5p | hsa-miR-7152-5p | hsa-miR-5590-5p |
| hsa-miR-3150b-3p | hsa-miR-328-3p | hsa-miR-7158-5p | hsa-miR-676-3p |
| hsa-miR-3181 | hsa-miR-7110-3p | hsa-miR-6883-5p | hsa-miR-4742-5p |
| hsa-miR-3182 | hsa-miR-6820-3p | hsa-miR-3613-3p | hsa-miR-421 |
| hsa-miR-3183 | hsa-miR-6809-3p | hsa-miR-3928-5p | hsa-miR-4764-3p |
| hsa-miR-5581-5p | hsa-miR-1287-3p | hsa-miR-6842-3p | hsa-miR-196a-3p |
| hsa-miR-1224-5p | hsa-miR-3614-3p | hsa-miR-641 | hsa-miR-323b-5p |
| hsa-miR-300 | hsa-miR-5197-5p | hsa-miR-640 | hsa-miR-4789-3p |
| hsa-miR-3160-3p | hsa-miR-4255 | hsa-miR-643 | hsa-miR-3942-3p |

14. The computer-implemented method of claim 13, wherein the set of non-coding RNAs further comprises one or more non-coding RNAs from Table 13.
TABLE 13-1

| | | | |
|---|---|---|---|
| hsa-miR-6769a-3p | hsa-miR-4659a-3p | hsa-miR-6807-3p | hsa-miR-3194-5p |
| hsa-miR-548s | hsa-miR-526b-5p | hsa-miR-657 | hsa-miR-3162-5p |
| hsa-miR-7106-3p | hsa-miR-5579-5p | hsa-miR-548am-3p | hsa-miR-3156-5p |
| hsa-miR-506-3p | hsa-miR-32-3p | hsa-miR-4659b-3p | hsa-miR-548ai, |
| | | | hsa-miR-570-5p |
| hsa-miR-5009-3p | hsa-miR-3622b-3p | hsa-miR-6895-3p | hsa-miR-579-3p |
| hsa-miR-1537-3p | hsa-miR-4748 | hsa-miR-129-1-3p | hsa-miR-937-3p |
| hsa-miR-4783-5p | hsa-miR-494-5p | hsa-miR-4517 | hsa-miR-3157-5p |
| hsa-miR-203b-5p | hsa-miR-4744 | hsa-miR-1250-3p | hsa-miR-450a-1-3p |
| hsa-miR-548ah-5p | hsa-miR-5693 | hsa-miR-92a-1-5p | hsa-miR-873-5p |
| hsa-miR-8085 | hsa-miR-5690 | hsa-miR-582-5p | hsa-miR-4735-3p |
| hsa-miR-3973 | hsa-miR-5691 | hsa-miR-125b-2-3p | hsa-miR-450a-2-3p |
| hsa-miR-3972 | hsa-miR-5696 | hsa-miR-6800-3p | hsa-miR-921 |
| hsa-miR-8080 | hsa-miR-5697 | hsa-miR-4769-5p | hsa-miR-922 |
| hsa-miR-3974 | hsa-miR-5694 | hsa-miR-6835-3p | hsa-miR-924 |
| hsa-miR-3977 | hsa-miR-5695 | hsa-miR-1178-5p | hsa-miR-4725-5p |
| hsa-miR-8083 | hsa-miR-5698 | hsa-miR-6720-3p | hsa-miR-6783-5p |
| hsa-miR-3978 | hsa-miR-33b-5p | hsa-miR-4740-3p | hsa-miR-155-5p |
| hsa-miR-383-3p | hsa-miR-4501 | hsa-miR-6803-3p | hsa-miR-125b-1-3p |
| hsa-miR-8088 | hsa-miR-6734-5p | hsa-miR-4418 | hsa-miR-18b-3p |
| hsa-miR-8089 | hsa-miR-4772-5p | hsa-miR-4417 | hsa-miR-6768-5p |
| hsa-miR-3940-3p | hsa-miR-302c-5p | hsa-miR-4419a | hsa-miR-6874-3p |
| hsa-miR-218-1-3p | hsa-miR-6782-3p | hsa-miR-3160-5p | hsa-miR-1229-5p |
| hsa-miR-200b-5p | hsa-miR-212-3p | hsa-miR-325 | hsa-miR-3690 |
| hsa-miR-519d-5p | hsa-miR-1913 | hsa-miR-6878-3p | hsa-miR-4653-3p |
| hsa-miR-7977 | hsa-miR-1912 | hsa-miR-4668-5p | hsa-miR-3663-5p |
| hsa-miR-4664-3p | hsa-miR-26a-1-3p | hsa-miR-6127 | hsa-miR-6794-3p |
| hsa-miR-376a-2-5p | hsa-miR-518c-5p | hsa-miR-938 | hsa-miR-6728-3p |
| hsa-miR-6760-5p | hsa-miR-3691-3p | hsa-miR-3617-3p | hsa-miR-6866-3p |

TABLE 13-2

| | | | |
|---|---|---|---|
| hsa-miR-4680-3p | hsa-miR-1185-1-3p | hsa-miR-6129 | hsa-miR-5196-5p |
| hsa-miR-936 | hsa-miR-103a-2-5p | hsa-miR-6831-3p | hsa-miR-1256 |
| hsa-miR-4687-5p | hsa-miR-134-3p | hsa-miR-1226-5p | hsa-miR-379-5p |
| hsa-miR-6783-3p | hsa-miR-6832-3p | hsa-miR-3180-5p | hsa-miR-6514-3p |
| hsa-miR-4738-5p | hsa-miR-2113 | hsa-miR-6886-5p | hsa-miR-6507-5p |
| hsa-miR-6759-5p | hsa-miR-8086 | hsa-miR-5582-3p | hsa-miR-6732-3p |
| hsa-miR-3664-3p | hsa-miR-8087 | hsa-miR-4684-3p | hsa-miR-135b-5p |
| hsa-miR-4524a-5p | hsa-miR-588 | hsa-miR-512-3p | hsa-miR-6797-3p |
| hsa-miR-6819-3p | hsa-miR-6754-3p | hsa-miR-6862-5p | hsa-miR-6786-3p |
| hsa-miR-3606-5p | hsa-miR-3607-3p | hsa-miR-502-5p | hsa-miR-208a-5p |
| hsa-miR-182-5p | hsa-miR-8081 | hsa-miR-542-3p | hsa-miR-6888-3p |
| hsa-miR-183-3p | hsa-miR-4724-3p | hsa-miR-423-3p | hsa-miR-3200-3p |
| hsa-miR-6846-3p | hsa-miR-100-3p | hsa-miR-3667-3p | hsa-miR-6509-3p |
| hsa-miR-548a-5p | hsa-miR-6880-3p | hsa-miR-935 | hsa-miR-4662a-3p |
| hsa-miR-891a-3p | hsa-miR-3976 | hsa-miR-5007-5p | hsa-miR-3944-3p |
| hsa-miR-1914-5p | hsa-miR-653-3p | hsa-miR-4445-3p | hsa-miR-3529-5p |
| hsa-miR-590-5p | hsa-miR-5586-3p | hsa-miR-4685-5p | hsa-miR-6839-3p |
| hsa-miR-511-5p | hsa-miR-223-5p | hsa-miR-3192-3p | hsa-miR-6715b-5p |
| hsa-miR-1827 | hsa-miR-4750-3p | hsa-miR-659-3p | hsa-miR-6731-3p |
| hsa-miR-889-5p | hsa-miR-6757-3p | hsa-miR-6505-5p | hsa-miR-199a-3p, |
| | | | or hsa-miR-199b-3p |
| hsa-miR-6891-3p | hsa-miR-6773-3p | hsa-miR-3927-3p | hsa-miR-1255a |
| hsa-miR-548t-5p | hsa-miR-6751-5p | hsa-miR-6738-3p | hsa-miR-548ar-5p |

15. The computer-implemented method of claim 14, wherein the set of non-coding RNAs further comprises one or more non-coding RNAs from Table 14.
TABLE 14-1

| | | | |
|---|---|---|---|
| hsa-miR-4747-3p | hsa-miR-378f | hsa-miR-4797-5p | hsa-miR-4470 |
| hsa-miR-345-5p | hsa-miR-378g | hsa-miR-4716-5p | hsa-miR-4473 |
| hsa-miR-379-3p | hsa-miR-2276-3p | hsa-miR-6884-3p | hsa-miR-761 |
| hsa-miR-6854-5p | hsa-miR-378e | hsa-miR-6802-3p | hsa-miR-4475 |
| hsa-miR-548ba | hsa-miR-378j | hsa-miR-548g-5p, | hsa-miR-764 |
| | | hsa-miR-548x-5p, | |
| | | or hsa-miR-548aj-5p | |
| hsa-miR-4676-3p | hsa-miR-378h | hsa-miR-5091 | hsa-miR-765 |
| hsa-miR-4775 | hsa-miR-4653-5p | hsa-miR-5092 | hsa-miR-555 |
| hsa-miR-5689 | hsa-miR-24-2-5p | hsa-miR-6837-3p | hsa-miR-4679 |
| hsa-miR-5688 | hsa-miR-1278 | hsa-miR-5094 | hsa-miR-4678 |
| hsa-miR-4770 | hsa-miR-1279 | hsa-miR-5095 | hsa-miR-4673 |
| hsa-miR-4771 | hsa-miR-1270 | hsa-miR-5096 | hsa-miR-4672 |
| hsa-miR-5685 | hsa-miR-1272 | hsa-miR-576-3p | hsa-miR-6774-3p |
| hsa-miR-5684 | hsa-miR-6784-3p | hsa-miR-33a-5p | hsa-miR-543 |
| hsa-miR-5687 | hsa-miR-1276 | hsa-miR-4727-3p | hsa-miR-371a-3p |
| hsa-miR-4701-5p | hsa-miR-4303 | hsa-miR-378d | hsa-miR-6829-3p |
| hsa-miR-5680 | hsa-miR-4302 | hsa-miR-99a-3p | hsa-miR-6801-5p |
| hsa-miR-5683 | hsa-miR-4301 | hsa-miR-548h-5p | hsa-miR-6511b-3p |
| hsa-miR-4787-3p | hsa-miR-4300 | hsa-miR-548as-3p | hsa-miR-20b-3p |
| hsa-miR-34c-5p | hsa-miR-4307 | hsa-miR-4649-5p | hsa-miR-1539 |
| hsa-miR-556-5p | hsa-miR-4305 | hsa-miR-4758-3p | hsa-miR-1538 |
| hsa-miR-1306-3p | hsa-miR-4304 | hsa-miR-378i | hsa-miR-498 |
| hsa-miR-4760-5p | hsa-miR-4309 | hsa-miR-6857-5p | hsa-miR-496 |
| hsa-miR-1253 | hsa-miR-4308 | hsa-miR-128-2-5p | hsa-miR-492 |
| hsa-miR-5093 | hsa-miR-7112-5p | hsa-miR-6506-5p | hsa-miR-3622a-3p |
| hsa-miR-4272 | hsa-miR-6822-5p | hsa-miR-4714-5p | hsa-miR-134-5p |
| hsa-miR-516b-3p, | hsa-miR-2110 | hsa-miR-6833-3p | hsa-miR-525-5p |
| or hsa-miR-516a-3p | | | |
| hsa-miR-506-5p | hsa-miR-4755-5p | hsa-miR-194-3p | hsa-miR-6894-3p |
| hsa-miR-145-3p | hsa-miR-6507-3p | hsa-miR-3616-3p | hsa-miR-518b |
| hsa-miR-449c-3p | hsa-miR-6726-3p | hsa-miR-1277-5p | hsa-miR-3155b |
| hsa-miR-1292-5p | hsa-miR-135b-3p | hsa-miR-892b | hsa-miR-3155a |

TABLE 14-2

| | | | |
|---|---|---|---|
| hsa-miR-6790-3p | hsa-miR-520b | hsa-miR-892a | hsa-miR-584-3p |
| hsa-miR-190a-Sp | hsa-miR-520e | hsa-miR-4711-3p | hsa-miR-6787-3p |
| hsa-miR-6777-3p | hsa-miR-3140-5p | hsa-miR-7844-5p | hsa-miR-5588-3p |
| hsa-miR-6858-3p | hsa-miR-520h | hsa-miR-2276-5p | hsa-miR-3192-5p |
| hsa-miR-7113-5p | hsa-miR-6868-5p | hsa-miR-1266-3p | hsa-miR-659-5p |
| hsa-miR-1234-3p | hsa-miR-6888-5p | hsa-miR-1237-3p | hsa-miR-6764-3p |
| hsa-miR-1273a | hsa-miR-5681a | hsa-miR-4329 | hsa-miR-627-5p |
| hsa-miR-3912-5p | hsa-miR-5681b | hsa-miR-1257 | hsa-miR-5692a |
| hsa-miR-1273c | hsa-miR-1305 | hsa-miR-1254 | hsa-miR-5692c |
| hsa-miR-1273d | hsa-miR-4536-3p | hsa-miR-5197-3p | hsa-miR-5692b |
| hsa-miR-1273e | hsa-miR-1302 | hsa-miR-4321 | hsa-miR-6738-5p |
| hsa-miR-1273f | hsa-miR-4662a-5p | hsa-miR-4320 | hsa-miR-6882-5p |
| hsa-miR-6736-3p | hsa-miR-181c-5p | hsa-miR-4323 | hsa-miR-3128 |
| hsa-miR-758-5p | hsa-miR-4802-3p | hsa-miR-4322 | hsa-miR-384 |
| hsa-miR-4279 | hsa-miR-628-3p | hsa-miR-4325 | hsa-miR-3122 |
| hsa-miR-3613-5p | hsa-miR-365a-3p, | hsa-miR-4327 | hsa-miR-3123 |
| | or hsa-miR-365b-3p | | |
| hsa-miR-7515 | hsa-miR-6854-3p | hsa-miR-4326 | hsa-miR-579-5p |
| hsa-miR-2355-5p | hsa-miR-548ay-3p | hsa-miR-7112-3p | hsa-miR-4252 |
| hsa-miR-3616-5p | hsa-miR-4650-5p | hsa-miR-143-5p | hsa-miR-3125 |
| hsa-miR-5006-5p | hsa-miR-509-3-5p | hsa-miR-4645-3p | hsa-miR-6790-5p |
| hsa-miR-4703-5p | hsa-miR-625-5p | hsa-miR-6750-3p | hsa-miR-873-3p |
| hsa-miR-2114-3p | hsa-miR-4782-5p | hsa-miR-16-2-3p | hsa-miR-5186 |
| hsa-miR-3202 | hsa-miR-548ao-3p | hsa-miR-548c-5p, | hsa-miR-7978 |
| | | hsa-miR-548o-5p, | |
| | | or hsa-miR-548am-5p | |
| hsa-miR-3201 | hsa-miR-6772-3p | hsa-miR-6868-3p | hsa-miR-7975 |
| hsa-miR-1255b-5p | hsa-miR-1301-5p | hsa-miR-3074-5p | hsa-miR-7974 |
| hsa-miR-3620-3p | hsa-miR-3145-3p | hsa-miR-4747-5p | hsa-miR-944 |
| hsa-miR-4794 | hsa-miR-6808-3p | hsa-miR-605-5p | hsa-miR-7976 |
| hsa-miR-4791 | hsa-miR-7-5p | hsa-miR-1322 | hsa-miR-943 |
| hsa-miR-624-5p | hsa-miR-3658 | hsa-miR-1323 | hsa-miR-7973 |
| hsa-miR-4423-3p | hsa-miR-3659 | hsa-miR-1321 | hsa-miR-2681-3p |

TABLE 14-3

| | | | |
|---|---|---|---|
| hsa-miR-373-3p | hsa-miR-4760-3p | hsa-miR-1324 | hsa-miR-6875-3p |
| hsa-miR-6501-3p | hsa-miR-3655 | hsa-miR-3677-3p | hsa-miR-6768-3p |
| hsa-miR-329-5p | hsa-miR-3657 | hsa-miR-370-3p | hsa-miR-4639-5p |
| hsa-miR-3925-5p | hsa-miR-4700-5p | hsa-miR-625-3p | hsa-miR-5583-3p |
| hsa-miR-7849-3p | hsa-miR-3651 | hsa-miR-8054 | hsa-miR-4638-3p |
| hsa-miR-4428 | hsa-miR-3652 | hsa-miR-548ay-5p | hsa-miR-141-5p |
| hsa-miR-4422 | hsa-miR-522-3p | hsa-miR-6772-5p | hsa-miR-708-3p |
| hsa-miR-4420 | hsa-miR-6719-3p | hsa-miR-6796-3p | hsa-miR-520d-3p |
| hsa-miR-4421 | hsa-miR-518d-3p | hsa-miR-550b-2-5p | hsa-miR-4269 |
| hsa-miR-4426 | hsa-miR-2115-5p | hsa-miR-606 | hsa-miR-7854-3p |
| hsa-miR-4427 | hsa-miR-6771-3p | hsa-miR-31-5p | hsa-miR-3689a-3p |
| hsa-miR-4424 | hsa-miR-4446-5p | hsa-miR-7154-3p | hsa-miR-193b-5p |
| hsa-miR-4425 | hsa-miR-6514-5p | hsa-miR-513b-3p | hsa-miR-3158-3p |
| hsa-miR-4731-5p | hsa-miR-769-5p | hsa-miR-3670 | hsa-miR-515-3p |
| hsa-miR-675-5p | hsa-miR-424-3p | hsa-miR-519c-5p, | hsa-miR-6780a-5p |
| | | hsa-miR-523-5p, | |
| | | hsa-miR-518e-5p, | |
| | | hsa-miR-522-5p, | |
| | | hsa-miR-519a-5p, | |
| | | or hsa-miR-519b-5p | |
| hsa-miR-3173-5p | hsa-miR-3913-3p | hsa-miR-3674 | hsa-miR-3074-3p |
| hsa-miR-376a-3p | hsa-miR-758-3p | hsa-miR-6128 | hsa-miR-3529-3p |
| hsa-miR-195-3p | hsa-miR-6500-3p | hsa-miR-4804-5p | hsa-miR-3907 |
| hsa-miR-6876-5p | hsa-miR-3129-3p | hsa-miR-6849-3p | hsa-miR-3908 |
| hsa-miR-153-3p | hsa-miR-6779-3p | hsa-miR-330-5p | hsa-miR-3909 |
| hsa-miR-655-5p | hsa-miR-2277-5p | hsa-miR-8066 | hsa-miR-4273 |
| hsa-miR-5590-3p | hsa-miR-147a | hsa-miR-34a-3p | hsa-miR-3614-5p |
| hsa-miR-4742-3p | hsa-miR-500b-5p | hsa-miR-574-3p | hsa-miR-4277 |
| hsa-miR-4671-3p | hsa-miR-6747-5p | hsa-miR-4686 | hsa-miR-4276 |
| hsa-miR-4789-5p | hsa-miR-6853-3p | hsa-miR-4682 | hsa-miR-4275 |
| hsa-miR-4693-5p | hsa-miR-4795-5p | hsa-miR-4683 | hsa-miR-4274 |
| hsa-miR-4757-5p | hsa-miR-4703-3p | hsa-miR-4681 | hsa-miR-1184 |
| hsa-miR-3150b-5p | hsa-miR-4726-3p | hsa-miR-769-3p | hsa-miR-4278 |

TABLE 14-4

| | | | |
|---|---|---|---|
| hsa-miR-3189-5p | hsa-miR-5682 | hsa-miR-3922-5p | hsa-miR-1182 |
| hsa-miR-410-3p | hsa-miR-656-5p | hsa-miR-34b-3p | hsa-miR-1183 |
| hsa-miR-302f | hsa-miR-3591-5p | hsa-miR-7158-3p | hsa-miR-1181 |
| hsa-miR-302e | hsa-miR-550a-3p | hsa-miR-589-3p | hsa-miR-370-5p |
| hsa-miR-1298-5p | hsa-miR-6859-5p | hsa-miR-3129-5p | hsa-miR-6881-5p |
| hsa-miR-4524b-3p | hsa-miR-6737-5p | hsa-miR-4690-3p | hsa-miR-548z, or |
| | | | hsa-miR-548h-3p |
| hsa-miR-302d-3p | hsa-miR-7160-3p | hsa-miR-8064 | hsa-miR-487a-5p |
| hsa-miR-508-5p | hsa-miR-4445-5p | hsa-miR-7111-3p | hsa-miR-6736-5p |
| hsa-miR-4661-5p | hsa-miR-202-5p | hsa-miR-1236-5p | hsa-miR-7152-3p |
| hsa-miR-527, | hsa-miR-6792-5p | hsa-miR-4720-3p | hsa-miR-3187-3p |
| or hsa-miR-518a-5p | | | |
| hsa-miR-767-3p | hsa-miR-1470 | hsa-miR-3928-3p | hsa-miR-539-5p |
| hsa-miR-4776-3p | hsa-miR-1471 | hsa-miR-500b-3p | hsa-miR-138-2-3p |
| hsa-miR-1245b-3p | hsa-miR-4662b | hsa-miR-6853-5p | hsa-miR-887-5p |
| hsa-miR-7850-5p | hsa-miR-4652-5p | hsa-miR-106a-3p | hsa-miR-7154-5p |
| hsa-miR-7851-3p | hsa-miR-6857-3p | hsa-miR-4795-3p | hsa-miR-210-3p |
| hsa-miR-3683 | hsa-miR-591 | hsa-miR-6770-5p | hsa-miR-3610 |
| hsa-miR-3687 | hsa-miR-329-3p | hsa-miR-1304-3p | hsa-miR-3611 |
| hsa-miR-3686 | hsa-miR-592 | hsa-miR-6859-3p | hsa-miR-3612 |
| hsa-miR-3685 | hsa-miR-595 | hsa-miR-628-5p | hsa-miR-593-5p |
| hsa-miR-3684 | hsa-miR-3184-3p | hsa-miR-4728-3p | hsa-miR-3615 |
| hsa-miR-1208 | hsa-miR-599 | hsa-miR-518f-5p | hsa-miR-3618 |
| hsa-miR-6776-5p | hsa-miR-6735-5p | hsa-miR-98-3p | hsa-miR-4804-3p |
| hsa-miR-1205 | hsa-miR-4440 | hsa-miR-202-3p | hsa-miR-6809-5p |
| hsa-miR-1204 | hsa-miR-4441 | hsa-miR-6792-3p | hsa-miR-1287-5p |
| hsa-miR-1206 | hsa-miR-4443 | hsa-miR-412-3p | hsa-miR-6849-5p |
| hsa-miR-1200 | hsa-miR-4444 | hsa-miR-6825-3p | hsa-miR-551a |
| hsa-miR-1203 | hsa-miR-4448 | hsa-miR-5188 | hsa-miR-1293 |
| hsa-miR-4310 | hsa-miR-759 | hsa-miR-4468 | hsa-miR-1290 |
| hsa-miR-4311 | hsa-miR-548aq-3p | hsa-miR-1199-3p | hsa-miR-26a-2-3p |
| hsa-miR-4312 | hsa-miR-578 | hsa-miR-3139 | hsa-miR-1297 |
| hsa-miR-4313 | hsa-miR-577 | hsa-miR-4464 | hsa-miR-1294 |
| hsa-miR-4314 | hsa-miR-6876-3p | hsa-miR-6734-3p | hsa-miR-190a-3p |

TABLE 14-5

| | | | |
|---|---|---|---|
| hsa-miR-504-3p | hsa-miR-655-3p | hsa-miR-4462 | hsa-miR-374b-3p |
| hsa-miR-4316 | hsa-miR-4733-5p | hsa-miR-4460 | hsa-miR-363-5p |
| hsa-miR-4317 | hsa-miR-4691-5p | hsa-miR-4461 | hsa-miR-4800-3p |
| hsa-miR-4318 | hsa-miR-5003-3p | hsa-miR-4669 | hsa-miR-372-5p |
| hsa-miR-4319 | hsa-miR-5187-5p | hsa-miR-513a-3p | hsa-miR-4769-3p |
| hsa-miR-6870-3p | hsa-miR-2278 | hsa-miR-548az-5p | hsa-miR-2053 |
| hsa-miR-548e-3p | hsa-miR-4671-5p | hsa-miR-4660 | hsa-miR-2052 |
| hsa-miR-891a-5p | hsa-miR-6752-3p | hsa-miR-4663 | hsa-miR-4670-5p |
| hsa-miR-3934-5p | hsa-miR-3682-5p | hsa-miR-559 | hsa-miR-2054 |
| hsa-miR-3606-3p | hsa-miR-301a-5p | hsa-miR-558 | hsa-miR-523-3p |
| hsa-miR-6788-5p | hsa-miR-3130-5p | hsa-miR-548aq-5p | hsa-miR-6770-3p |
| hsa-miR-218-5p | hsa-miR-519a-3p | hsa-miR-1185-5p | hsa-miR-6744-5p |
| hsa-miR-877-3p | hsa-miR-4713-3p | hsa-miR-554 | hsa-miR-6845-3p |
| hsa-miR-3688-3p | hsa-miR-4640-3p | hsa-miR-524-5p | hsa-miR-511-3p |
| hsa-miR-548g-3p | hsa-miR-410-5p | hsa-miR-553 | hsa-miR-651-3p |
| hsa-miR-598-3p | hsa-miR-3681-5p | hsa-miR-3621 | hsa-miR-518f-3p |
| hsa-miR-152-3p | hsa-miR-3680-3p | hsa-miR-9-3p | hsa-miR-4699-3p |
| hsa-miR-7162-3p | hsa-miR-4793-3p | hsa-miR-4733-3p | hsa-miR-1295b-5p |
| hsa-miR-664a-5p | hsa-miR-4524b-5p | hsa-miR-891b | hsa-miR-30b-3p |
| hsa-miR-874-3p | hsa-miR-24-1-5p | hsa-miR-676-5p | hsa-miR-7151-3p |
| hsa-miR-4765 | hsa-miR-6816-3p | hsa-miR-6834-5p | hsa-miR-1909-5p |
| hsa-miR-4767 | hsa-miR-1245b-5p | hsa-miR-4764-5p | hsa-miR-4487 |
| hsa-miR-3622b-5p | hsa-miR-302d-5p | hsa-miR-490-5p | hsa-miR-4480 |
| hsa-miR-6811-5p | hsa-miR-508-3p | hsa-miR-3682-3p | hsa-miR-876-5p |
| hsa-miR-499b-5p | hsa-miR-3692-3p | hsa-miR-608 | hsa-miR-4483 |
| hsa-miR-4772-3p | hsa-miR-8082 | hsa-miR-5087 | hsa-miR-27b-5p |
| hsa-miR-212-5p | hsa-miR-6499-5p | hsa-miR-4723-3p | hsa-miR-4489 |
| hsa-miR-518c-3p | hsa-miR-6810-5p | hsa-miR-7846-3p | hsa-miR-6780b-3p |
| hsa-miR-422a | hsa-miR-449b-3p | hsa-miR-4677-3p | hsa-miR-4648 |
| hsa-miR-548c-3p | hsa-miR-185-3p | hsa-miR-6755-5p | hsa-miR-513a-5p |
| hsa-miR-3679-3p | hsa-miR-6512-5p | hsa-miR-1537-5p | hsa-miR-6728-5p |
| hsa-miR-6818-3p | hsa-miR-6758-3p | hsa-miR-3680-5p | hsa-miR-4642 |
| hsa-miR-6754-5p | hsa-miR-6513-3p | hsa-miR-1303 | hsa-miR-6516-3p |
| hsa-miR-6864-3p | hsa-miR-4649-3p | hsa-miR-95-5p | hsa-miR-3150a-5p |

TABLE 14-6

| | | | |
|---|---|---|---|
| hsa-miR-129-5p | hsa-miR-6813-5p | hsa-miR-3119 | hsa-miR-4641 |
| hsa-miR-6823-3p | hsa-miR-6812-3p | hsa-miR-3118 | hsa-miR-4647 |
| hsa-miR-1468-5p | hsa-miR-5589-3p | hsa-miR-3116 | hsa-miR-4644 |
| hsa-miR-580-5p | hsa-miR-374c-3p | hsa-miR-3115 | hsa-miR-3186-5p |
| hsa-miR-548ak | hsa-miR-1263 | hsa-miR-412-5p | hsa-miR-6746-3p |
| hsa-miR-4999-5p | hsa-miR-1262 | hsa-miR-889-3p | hsa-miR-5009-5p |
| hsa-miR-548an | hsa-miR-1261 | hsa-miR-6748-3p | hsa-miR-6851-3p |
| hsa-miR-548al | hsa-miR-1267 | hsa-miR-4664-5p | hsa-miR-203b-3p |
| hsa-miR-548ac | hsa-miR-1265 | hsa-miR-490-3p | hsa-miR-5008-3p |
| hsa-miR-548ab | hsa-miR-1264 | hsa-miR-6760-3p | hsa-miR-152-5p |
| hsa-miR-548ag | hsa-miR-802 | hsa-miR-6499-3p | hsa-miR-7843-3p |
| hsa-miR-552-3p | hsa-miR-6742-3p | hsa-miR-517c-3p | hsa-miR-6818-5p |
| hsa-miR-548ax | hsa-miR-3934-3p | hsa-miR-376b-5p | hsa-miR-199b-5p |
| hsa-miR-488-5p | hsa-miR-3177-3p | hsa-miR-5011-5p | hsa-miR-622 |
| hsa-miR-4633-3p | hsa-miR-1911-5p | hsa-miR-3117-3p | hsa-miR-621 |
| hsa-miR-450b-3p | hsa-miR-1185-2-3p | hsa-miR-6810-3p | hsa-miR-620 |
| hsa-miR-548aw | hsa-miR-6788-3p | hsa-miR-340-3p | hsa-miR-1255b-2-3p |
| hsa-miR-216b-5p | hsa-miR-877-5p | hsa-miR-4738-3p | hsa-miR-626 |
| hsa-miR-4540 | hsa-miR-4727-5p | hsa-miR-6513-5p | hsa-miR-1296-5p |
| hsa-miR-4433b-5p | hsa-miR-489-5p | hsa-miR-6740-3p | hsa-miR-4693-3p |
| hsa-miR-1250-5p | hsa-miR-3688-5p | hsa-miR-548at-3p | hsa-miR-448 |
| hsa-miR-4659b-5p | hsa-miR-4680-5p | hsa-miR-4774-5p | hsa-miR-5000-3p |
| hsa-miR-6775-3p | hsa-miR-505-5p | hsa-miR-1243 | hsa-miR-4481 |
| hsa-miR-545-5p | hsa-miR-6871-3p | hsa-miR-1244 | hsa-miR-6855-3p |
| hsa-miR-4788 | hsa-miR-380-3p | hsa-miR-3927-5p | hsa-miR-1298-3p |
| hsa-miR-4785 | hsa-miR-8071 | hsa-miR-1248 | hsa-miR-3127-3p |
| hsa-miR-4784 | hsa-miR-5010-5p | hsa-miR-6852-5p | hsa-miR-561-3p |
| hsa-miR-4780 | hsa-miR-6892-3p | hsa-miR-548ah-3p | hsa-miR-3134 |
| hsa-miR-3675-3p | hsa-miR-4643 | hsa-miR-501-3p | hsa-miR-3137 |
| hsa-miR-4717-3p | hsa-miR-499b-3p | hsa-miR-520a-5p | hsa-miR-3133 |
| hsa-miR-6835-5p | hsa-miR-6872-3p | hsa-miR-4766-5p | hsa-miR-3132 |
| hsa-miR-6828-5p | hsa-miR-200a-3p | hsa-miR-3177-5p | hsa-miR-513c-5p |
| hsa-miR-5699-3p | hsa-miR-181d-3p | hsa-miR-1180-3p | hsa-miR-7848-3p |

TABLE 14-7

| | | | |
|---|---|---|---|
| hsa-miR-6720-5p | hsa-miR-3646 | hsa-miR-4659a-5p | hsa-miR-3138 |
| hsa-miR-4722-3p | hsa-miR-8070 | hsa-miR-489-3p | hsa-miR-5194 |
| hsa-miR-4740-5p | hsa-miR-6767-5p | hsa-miR-382-3p | hsa-miR-5191 |
| hsa-miR-191-3p | hsa-miR-1269b | hsa-miR-4260 | hsa-miR-5190 |
| hsa-miR-8068 | hsa-miR-1269a | hsa-miR-4261 | hsa-miR-5193 |
| hsa-miR-604 | hsa-miR-520f-3p | hsa-miR-570-3p | hsa-miR-5192 |
| hsa-miR-4779 | hsa-miR-4694-3p | hsa-miR-4263 | hsa-miR-4524a-3p |
| hsa-miR-5004-5p | hsa-miR-525-3p | hsa-miR-4264 | hsa-miR-5011-3p |
| hsa-miR-1225-3p | hsa-miR-3671 | hsa-miR-4265 | hsa-miR-892c-5p |
| hsa-miR-4435 | hsa-miR-548ap-5p | hsa-miR-4266 | hsa-miR-4776-5p |
| hsa-miR-4434 | hsa-miR-302a-3p | hsa-miR-4267 | hsa-miR-7107-3p |
| hsa-miR-4437 | hsa-miR-4666b | hsa-miR-4268 | hsa-miR-1247-5p |
| hsa-miR-3124-5p | hsa-miR-875-5p | hsa-miR-154-3p | hsa-miR-3190-5p |
| hsa-miR-4431 | hsa-miR-4799-3p | hsa-miR-6871-5p | hsa-miR-129-2-3p |
| hsa-miR-4430 | hsa-miR-550a-3-5p | hsa-miR-6079 | hsa-miR-219b-5p |
| hsa-miR-4724-5p | hsa-miR-6729-3p | hsa-miR-6827-3p | hsa-miR-6766-3p |
| hsa-miR-4432 | hsa-miR-6887-3p | hsa-miR-494-3p | hsa-miR-6740-5p |
| hsa-miR-601 | hsa-miR-6823-5p | hsa-miR-6892-5p | hsa-miR-30c-2-3p |
| hsa-miR-1251-5p | hsa-miR-302b-3p | hsa-miR-1910-5p | hsa-miR-6776-3p |
| hsa-miR-6716-5p | hsa-miR-7156-5p | hsa-miR-1233-3p | hsa-miR-548at-5p |
| hsa-miR-600 | hsa-miR-208b-3p | hsa-miR-585-5p | hsa-miR-4801 |
| hsa-miR-346 | hsa-miR-153-5p | hsa-miR-3666 | hsa-miR-504-5p |
| hsa-miR-8062 | hsa-miR-8075 | hsa-miR-3661 | hsa-miR-668-5p |
| hsa-miR-3064-3p | hsa-miR-6730-3p | hsa-miR-3660 | hsa-miR-186-3p |
| hsa-miR-4668-3p | hsa-miR-5584-5p | hsa-miR-3662 | hsa-miR-653-5p |
| hsa-miR-4708-5p | hsa-miR-5585-3p | hsa-miR-6883-3p | hsa-miR-4712-3p |
| hsa-miR-377-5p | hsa-miR-4474-5p | hsa-miR-4469 | hsa-miR-3942-5p |
| hsa-miR-8061 | hsa-miR-7157-5p | hsa-miR-3668 | hsa-miR-5047 |
| hsa-miR-4282 | hsa-miR-6804-3p | hsa-miR-224-3p | hsa-miR-146b-3p |
| hsa-miR-4328 | hsa-miR-3120-5p | hsa-miR-5591-3p | hsa-miR-4697-3p |
| hsa-miR-4439 | hsa-miR-455-3p | hsa-miR-4743-3p | hsa-miR-3173-3p |
| hsa-miR-6889-3p | hsa-miR-3121-3p | hsa-miR-4465 | hsa-miR-1180-5p |
| hsa-miR-8084 | hsa-miR-4717-5p | hsa-miR-487b-5p | hsa-miR-7155-5p |

TABLE 14-8

| | | | |
|---|---|---|---|
| hsa-miR-4438 | hsa-miR-6504-5p | hsa-miR-6879-3p | hsa-miR-3916 |
| hsa-miR-1252-5p | hsa-miR-5699-5p | hsa-miR-875-3p | hsa-miR-3915 |
| hsa-miR-4796-5p | hsa-miR-149-5p | hsa-miR-4692 | hsa-miR-3914 |
| hsa-miR-4797-3p | hsa-miR-210-5p | hsa-miR-4696 | hsa-miR-3911 |
| hsa-miR-3678-5p | hsa-miR-6756-3p | hsa-miR-4698 | hsa-miR-3910 |
| hsa-miR-6836-5p | hsa-miR-580-3p | hsa-miR-5586-5p | hsa-miR-3919 |
| hsa-miR-548au-3p | hsa-miR-371b-5p | hsa-miR-6895-5p | hsa-miR-3918 |
| hsa-miR-6837-5p | hsa-miR-5089-5p | hsa-miR-19a-5p | hsa-miR-3191-5p |
| hsa-miR-1288-5p | hsa-miR-181c-3p | hsa-miR-4507 | hsa-miR-1193 |
| hsa-miR-3135a | hsa-miR-587 | hsa-miR-770-5p | hsa-miR-182-3p |
| hsa-miR-5007-3p | hsa-miR-583 | hsa-miR-7156-3p | hsa-miR-7162-5p |
| hsa-miR-33a-3p | hsa-miR-5088-3p | hsa-miR-6840-5p | hsa-miR-1197 |
| hsa-miR-548as-5p | hsa-miR-581 | hsa-miR-6873-5p | hsa-miR-874-5p |
| hsa-miR-1258 | hsa-miR-5581-3p | hsa-miR-548j-5p | hsa-miR-4790-5p |
| hsa-miR-433-5p | hsa-miR-6722-5p | hsa-miR-6715a-3p | hsa-miR-6793-5p |
| hsa-miR-432-3p | hsa-miR-6806-3p | hsa-miR-3605-3p | hsa-miR-3667-5p |
| hsa-miR-3162-3p | hsa-miR-4453 | hsa-miR-6817-3p | hsa-miR-1273h-5p |
| hsa-miR-211-5p | hsa-miR-4452 | hsa-miR-5585-5p | hsa-miR-1283 |
| hsa-miR-3157-3p | hsa-miR-4451 | hsa-miR-3654 | hsa-miR-548av-5p |
| hsa-miR-369-3p | hsa-miR-4450 | hsa-miR-4803 | hsa-miR-1910-3p |
| hsa-miR-4745-3p | hsa-miR-4457 | hsa-miR-6814-3p | hsa-miR-517-5p |
| hsa-miR-6503-3p | hsa-miR-4456 | hsa-miR-3121-5p | hsa-miR-585-3p |
| hsa-miR-6723-5p | hsa-miR-4455 | hsa-miR-4749-3p | hsa-miR-6877-3p |
| hsa-miR-3692-5p | hsa-miR-4458 | hsa-miR-6511a-3p | hsa-miR-3609 |
| hsa-miR-939-3p | hsa-miR-568 | hsa-miR-3650 | hsa-miR-548o-3p |
| hsa-miR-6874-5p | hsa-miR-563 | hsa-miR-323b-3p | hsa-miR-6890-5p |
| hsa-miR-4666a-3p | hsa-miR-6165 | hsa-miR-4715-5p | hsa-miR-1284 |
| hsa-miR-519e-3p | hsa-miR-377-3p | hsa-miR-5088-5p | hsa-miR-1286 |
| hsa-miR-378b | hsa-miR-503-3p | hsa-miR-4482-3p | hsa-miR-1281 |
| hsa-miR-378c | hsa-miR-1252-3p | hsa-miR-4471 | hsa-miR-200c-5p |
| hsa-miR-6830-3p | hsa-miR-1282 | hsa-miR-1289 | hsa-miR-192-3p |

16. The computer-implemented method of claim 1, wherein each individual of the second group either (1) is a healthy individual or (2) has non-malignant breast disease or a cancer that is not breast cancer.

17. The computer-implemented method of claim 1, wherein each individual of the second group has non-malignant breast disease.

18. The computer-implemented method of claim 17, wherein each individual of the second group has a breast disease that is independently selected from the group consisting of mastitis, fat necrosis, breast cyst, papillary apocrine changes, epithelial-related calcifications, mild epithelial hyperplasia, mammary duct ectasia, periductal ectasia, non-sclerosing adenosis, periductal fibrosis, ductal hyperplasia, sclerosing adenosis, radial scar, intraductal papilloma, intraductal papillomatosis, atypical ductal hyperplasia, lobular hyperplasia, fibroadenoma, cystosarcoma phyllodes, lactating adenoma, and tubular adenoma.

19. The computer-implemented method of claim 1, wherein the classifier is a support vector machine (SVM) classifier.

20. The computer-implemented method of claim 19, wherein the SVM classifier has a linear kernel with L1 penalty for regularization.

21. The computer-implemented method of claim 1, wherein the classifier comprises two or more first level sub-classifiers and one or more second level sub-classifiers, wherein
the two or more first level sub-classifiers determine whether the expression levels of the set of non-coding RNAs in the biological sample collected from the subject align more closely with (A) or with (B), thereby outputting a result for each first level sub-classifier, and the one or more second level sub-classifiers combine results from the first level sub-classifiers, thereby determining one or more likelihood scores representing the likelihood that the subject has breast cancer.

22. The computer-implemented method of claim 21, wherein the two or more first level sub-classifiers are independently selected from: random forest, logistic regression, extra tree classifier, SVM, K-nearest neighbors, deep learning classifiers, and gradient boosting decision trees.

23. The computer-implemented method of claim 21, wherein the one or more second level sub-classifiers are independently selected from logistic regression, gradient boosting decision trees, deep learning classifiers, and SVM.

24. The computer-implemented method of claim 21, wherein the classifier comprises two or more second level sub-classifiers and a third level sub-classifier, wherein the third level sub-classifier combines the one or more likelihood scores determined by the second level sub-classifiers.

25. The computer-implemented method of claim 24, wherein the third level sub-classifier is gradient boosting decision trees.

26. The computer-implemented method of claim 1, wherein the biological sample is blood, plasma, serum, saliva, urine, cerebrospinal fluid, intraductal fluid, nipple discharge, a tissue specimen, or breast milk.

27. The computer-implemented method of claim 1, wherein the expression level of each non-coding RNA is determined by amplification, sequencing, microarray analysis, multiplex assay analysis, or a combination thereof.

28. The computer-implemented method of claim 1, wherein the expression level of each non-coding RNA is determined by an amplification technique selected from the group consisting of ligase chain reaction (LCR), polymerase chain reaction (PCR), reverse transcriptase PCR, quantitative PCR, real time PCR, isothermal amplification, and multiplex PCR.

29. The computer-implemented method of claim 27, wherein the expression level of each non-coding RNA is determined by a sequencing technique selected from the group consisting of dideoxy sequencing, reverse-termination sequencing, next generation sequencing, barcode sequencing, paired-end sequencing, pyrosequencing, deep sequencing, sequencing-by-synthesis, sequencing-by-hybridization, sequencing-by-ligation, single-molecule sequencing, and single molecule real-time sequencing-by-synthesis.

30. A method of treatment comprising:
a) determining, or having determined, expression levels of a set of non-coding RNAs in a biological sample obtained from a subject, wherein the set of non-coding RNAs comprises five or more non-coding RNAs selected from Table 1;
b) determining, or having determined, that the expression levels align more closely with (A) expression levels of the same set of non-coding RNAs in biological samples collected from a first group of individuals who have breast cancer, than with (B) expression levels of the same set of non-coding RNAs in biological samples collected from a second group of individuals who do not have breast cancer;
c) concluding that the subject has breast cancer; and
d) administering a treatment for breast cancer to the subject.
TABLE 1
hsa-miR-495-3p
hsa-miR-1976
hsa-miR-1973
hsa-miR-1972
hsa-miR-3065-3p
hsa-miR-4778-3p
hsa-miR-1914-3p
hsa-miR-3689a-5p,
hsa-miR-3689b-5p,
or hsa-miR-3689e
hsa-miR-4999-3p
hsa-miR-6873-3p

31. The method of claim 30, wherein the treatment for breast cancer comprises one or more of: surgery, radiation therapy, chemotherapy, immunotherapy and cell-based therapy.

32. The method of claim 30, wherein the conclusion that the subject has breast cancer is corroborated by one or more further diagnostic tests, prior to administering the treatment.

33. One or more machine-readable hardware storage devices for processing data to determine a likelihood score that a subject has breast cancer, by storing instructions that are executable by one or more data processing devices to perform operations comprising:
inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals who have breast cancer, or with (B) expression levels of the set of non-coding RNAs in biological samples collected from a second group of individuals who do not have breast cancer; and
determining, by the one or more data processing devices based on application of the classifier, the likelihood score that the subject has breast cancer,
wherein the set of non-coding RNAs comprises five or more non-coding RNAs selected from Table 1.
TABLE 1
hsa-miR-495-3p
hsa-miR-1976
hsa-miR-1973
hsa-miR-1972
hsa-miR-3065-3p
hsa-miR-4778-3p
hsa-miR-1914-3p
hsa-miR-3689a-5p,
hsa-miR-3689b-5p,
or hsa-miR-3689e
hsa-miR-4999-3p
hsa-miR-6873-3p

34. A system comprising:
one or more data processing devices; and
one or more machine-readable hardware storage devices for processing data to determine a likelihood score that a subject has breast cancer, by storing instructions that are executable by the one or more data processing devices to perform operations comprising:
inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals, each of whom has breast cancer, or with (B) expression levels of the same set of non-coding RNAs in biological samples collected from a second group of individuals, none of whom has breast cancer; and
determining, by the one or more data processing devices based on application of the classifier, the likelihood score that the subject has breast cancer,
wherein the set of non-coding RNAs comprises five or more non-coding RNAs selected from Table 1.
TABLE 1
hsa-miR-495-3p
hsa-miR-1976
hsa-miR-1973
hsa-miR-1972
hsa-miR-3065-3p
hsa-miR-4778-3p
hsa-miR-1914-3p
hsa-miR-3689a-5p,
hsa-miR-3689b-5p,
or hsa-miR-3689e
hsa-miR-4999-3p
hsa-miR-6873-3p

35. A computer-implemented method for processing data in one or more data processing devices to determine a likelihood score that a subject has a benign breast disease, the method comprising:
inputting, into a classifier, data representing one or more values for a classifier parameter, wherein the one or more values represent expression levels of a set of non-coding RNAs in a biological sample collected from the subject, with the classifier being for determining a score indicating whether the expression levels align more closely with (A) expression levels of the same set of non-coding RNAs in biological samples collected from a first group of individuals, each of whom has a benign breast disease and does not have breast cancer, or with (B) expression levels of the same set of non-coding RNAs in biological samples collected from a second group of individuals, each of whom has breast cancer; and
determining, by the one or more data processing devices, based on application of the classifier, a likelihood score that the subject has a benign breast disease and does not have breast cancer,
wherein the set of non-coding RNAs comprises five or more non-coding RNAs selected from Table 1.
TABLE 1
hsa-miR-495-3p
hsa-miR-1976
hsa-miR-1973
hsa-miR-1972
hsa-miR-3065-3p
hsa-miR-4778-3p
hsa-miR-1914-3p
hsa-miR-3689a-5p,
hsa-miR-3689b-5p,
or hsa-miR-3689e
hsa-miR-4999-3p
hsa-miR-6873-3p
